SEQUENCE LISTING (310 sequences)

SEQ ID NO: 1 - DNA, 20 bp, Artificial Sequence
  Synthetic Construct
ccagctcgcaccgaatgtct

SEQ ID NO: 2 - DNA, 26 bp, Artificial Sequence
  Synthetic Construct
catcttgtcctgatgcgcgtgagtca

SEQ ID NO: 3 - DNA, 19 bp, Artificial Sequence
  Synthetic Construct
tttctgccaccgcgctgac

SEQ ID NO: 4 - DNA, 22 bp, Artificial Sequence
  Synthetic Construct
gcagcaagaaagcccttgaatg

SEQ ID NO: 5 - DNA, 30 bp, Artificial Sequence
  Synthetic Construct
acatttccatagcgacagcacgggcggaat

SEQ ID NO: 6 - DNA, 30 bp, Artificial Sequence
  Synthetic Construct
ggactttggccaagcttctaaatttgcgtc

SEQ ID NO: 7 - DNA, 19 bp, Artificial Sequence
  Synthetic Construct
cgtgcagtctccacgcact

SEQ ID NO: 8 - DNA, 23 bp, Artificial Sequence
  Synthetic Construct
tcgaatgcgcagcaccgggatag

SEQ ID NO: 9 - DNA, 28 bp, Artificial Sequence
  Synthetic Construct
ggctgtgtttttggtggcatcgattatc

SEQ ID NO: 10 - DNA, 27 bp, Artificial Sequence
  Synthetic Construct
tcccacttaaagacttggtgttcatcc

SEQ ID NO: 11 - DNA, 24 bp, Artificial Sequence
  Synthetic Construct
acgggccggtaatgcggatctgga

SEQ ID NO: 12 - DNA, 29 bp, Artificial Sequence
  Synthetic Construct
tattcgccagaatcacaatcgccacctgt

SEQ ID NO: 13 - DNA, 24 bp, Artificial Sequence
  Synthetic Construct
ccgcggcgaaattaagctcagcga

SEQ ID NO: 14 - DNA, 25 bp, Artificial Sequence
  Synthetic Construct
ccaaacagaccaatgctggagttag

SEQ ID NO: 15 - DNA, 29 bp, Artificial Sequence
  Synthetic Construct
attcgttgcactatcgttaactgaataca

SEQ ID NO: 16 - DNA, 23 bp, Artificial Sequence
  Synthetic Construct
ctgtaccgtcggactttccagac

SEQ ID NO: 17 - DNA, 19 bp, Artificial Sequence
  Synthetic Construct
gcgaccggcaggttgatct

SEQ ID NO: 18 - DNA, 25 bp, Artificial Sequence
  Synthetic Construct
catgtcgagataggaagtgtgctgc

SEQ ID NO: 19 - DNA, 20 bp, Artificial Sequence
  Synthetic Construct
cggaaccattcgctaaactc

SEQ ID NO: 20 - DNA, 21 bp, Artificial Sequence
  Synthetic Construct
aggcgcaactgtaagttaccg

SEQ ID NO: 21 - DNA, 28 bp, Artificial Sequence
  Synthetic Construct
gcttaattctcaatctatccccacgtat

SEQ ID NO: 22 - DNA, 26 bp, Artificial Sequence
  Synthetic Construct
ctccagataacgtagtggtttggctg

SEQ ID NO: 23 - DNA, 21 bp, Artificial Sequence
  Synthetic Construct
ctttcatgacgaccgcgtcgg

SEQ ID NO: 24 - DNA, 23 bp, Artificial Sequence
  Synthetic Construct
ctctagaaggactctcatcgagc

SEQ ID NO: 25 - DNA, 20 bp, Artificial Sequence
  Synthetic Construct
attttaaaggtagatgcggg

SEQ ID NO: 26 - DNA, 20 bp, Artificial Sequence
  Synthetic Construct
cgccctgtgatttatgttca

SEQ ID NO: 27 - DNA, 20 bp, Artificial Sequence
  Synthetic Construct
gttttatgcacccaggaagc

SEQ ID NO: 28 - DNA, 19 bp, Artificial Sequence
  Synthetic Construct
tctgctgcggccagtcata

SEQ ID NO: 29 - DNA, 20 bp, Artificial Sequence
  Synthetic Construct
gcggctgccagttttgataa

SEQ ID NO: 30 - DNA, 19 bp, Artificial Sequence
  Synthetic Construct
gtggctaagtgcagcaggc

SEQ ID NO: 31 - DNA, 28 bp, Artificial Sequence
  Synthetic Construct
cgttccactaacacacaagctgattcag

SEQ ID NO: 32 - DNA, 26 bp, Artificial Sequence
  Synthetic Construct
atctcggttgatttcttttcctcggg

SEQ ID NO: 33 - DNA, 26 bp, Artificial Sequence
  Synthetic Construct
acgctttccaatgtgcagtaccagta

SEQ ID NO: 34 - DNA, 26 bp, Artificial Sequence
  Synthetic Construct
tgcgatccagacgaaacgtctcatcg

SEQ ID NO: 35 - DNA, 24 bp, Artificial Sequence
  Synthetic Construct
gtgataccacttcacctcgggcaa

SEQ ID NO: 36 - DNA, 24 bp, Artificial Sequence
  Synthetic Construct
aatacatcgcgacggctttctgcc

SEQ ID NO: 37 - DNA, 23 bp, Artificial Sequence
  Synthetic Construct
agaattacctttgaaagtcaggc

SEQ ID NO: 38 - DNA, 26 bp, Artificial Sequence
  Synthetic Construct
gcttcaaagcctgtcggaattggttt

SEQ ID NO: 39 - DNA, 25 bp, Artificial Sequence
  Synthetic Construct
gggcatttaacgacgaaactggcta

SEQ ID NO: 40 - DNA, 26 bp, Artificial Sequence
  Synthetic Construct
gtgttcggtgaatatccaaggtacgc

SEQ ID NO: 41 - DNA, 26 bp, Artificial Sequence
  Synthetic Construct
atgttggtcccattaactctgaagaa

SEQ ID NO: 42 - DNA, 26 bp, Artificial Sequence
  Synthetic Construct
cacctgtttgagggtggatagcagta

SEQ ID NO: 43 - DNA, 26 bp, Artificial Sequence
  Synthetic Construct
gcagggcaagcagtatcattaatcac

SEQ ID NO: 44 - DNA, 32 bp, Artificial Sequence
  Synthetic Construct
aattaaatcagcaccaatcattatcttcttcc

SEQ ID NO: 45 - DNA, 25 bp, Artificial Sequence
  Synthetic Construct
aagctgctgaccagccagcgtctga

SEQ ID NO: 46 - DNA, 23 bp, Artificial Sequence
  Synthetic Construct
cggcgatttgctgatttcgctcg

SEQ ID NO: 47 - DNA, 26 bp, Artificial Sequence
  Synthetic Construct
tgcagtgctgccataaccatgagtga

SEQ ID NO: 48 - DNA, 25 bp, Artificial Sequence
  Synthetic Construct
agcgcagaagtggtcctgcaacttt

SEQ ID NO: 49 - DNA, 29 bp, Artificial Sequence
  Synthetic Construct
cagtacggaatctttcgtattcatcagga

SEQ ID NO: 50 - DNA, 25 bp, Artificial Sequence
  Synthetic Construct
cagttcgggaagtgcaatacctgca

SEQ ID NO: 51 - DNA, 25 bp, Artificial Sequence
  Synthetic Construct
aattgagcaagcgatttcgggctgt

SEQ ID NO: 52 - DNA, 24 bp, Artificial Sequence
  Synthetic Construct
cgtttagaacgatgccgccatcct

SEQ ID NO: 53 - DNA, 24 bp, Artificial Sequence
  Synthetic Construct
ccttggacggaaatgtagctggca

SEQ ID NO: 54 - DNA, 25 bp, Artificial Sequence
  Synthetic Construct
aatcacatggttgacacctgctgtg

SEQ ID NO: 55 - DNA, 24 bp, Artificial Sequence
  Synthetic Construct
ggaaatctaacgagagagcatgct

SEQ ID NO: 56 - DNA, 19 bp, Artificial Sequence
  Synthetic Construct
cgatgcgtgacacccaggc

SEQ ID NO: 57 - DNA, 22 bp, Artificial Sequence
  Synthetic Construct
ctgtcgagatcaagcctgccga

SEQ ID NO: 58 - DNA, 23 bp, Artificial Sequence
  Synthetic Construct
acaaattgattgcccagctcggt

SEQ ID NO: 59 - DNA, 21 bp, Artificial Sequence
  Synthetic Construct
aatcagcggcgcacgatcttt

SEQ ID NO: 60 - DNA, 23 bp, Artificial Sequence
  Synthetic Construct
aatgctcgctgcaccggtggtat

SEQ ID NO: 61 - DNA, 21 bp, Artificial Sequence
  Synthetic Construct
aaatctgcaacgagctttggg

SEQ ID NO: 62 - DNA, 28 bp, Artificial Sequence
  Synthetic Construct
gtttataagtgggtaaaccgtgaatatc

SEQ ID NO: 63 - DNA, 26 bp, Artificial Sequence
  Synthetic Construct
ttcgtgtcactttaattcaccaagat

SEQ ID NO: 64 - DNA, 21 bp, Artificial Sequence
  Synthetic Construct
aaagccatgcgtctgacatct

SEQ ID NO: 65 - DNA, 22 bp, Artificial Sequence
  Synthetic Construct
aagctccaacatgaagatggct

SEQ ID NO: 66 - DNA, 24 bp, Artificial Sequence
  Synthetic Construct
agatggcaaagatattcaactaac

SEQ ID NO: 67 - DNA, 23 bp, Artificial Sequence
  Synthetic Construct
agtgccatcttgcaaatggcgat

SEQ ID NO: 68 - DNA, 32 bp, Artificial Sequence
  Synthetic Construct
tgcaattggtgtgttagtggatcgtcatgata

SEQ ID NO: 69 - DNA, 23 bp, Artificial Sequence
  Synthetic Construct
cagtggatggtggacgatcgggt

SEQ ID NO: 70 - DNA, 26 bp, Artificial Sequence
  Synthetic Construct
ttgtggtgatttatctgcgggatact

SEQ ID NO: 71 - DNA, 24 bp, Artificial Sequence
  Synthetic Construct
cgccagttaatagtttgcgcaacg

SEQ ID NO: 72 - DNA, 24 bp, Artificial Sequence
  Synthetic Construct
aaagcggttagctccttcggtcct

SEQ ID NO: 73 - DNA, 23 bp, Artificial Sequence
  Synthetic Construct
cgttcagtacaatgcggccgtta

SEQ ID NO: 74 - DNA, 22 bp, Artificial Sequence
  Synthetic Construct
ctgcgggaacggttataactgc

SEQ ID NO: 75 - DNA, 23 bp, Artificial Sequence
  Synthetic Construct
gcacccgatatactttctttgcc

SEQ ID NO: 76 - DNA, 21 bp, Artificial Sequence
  Synthetic Construct
cgccgacaatcaaatcatcct

SEQ ID NO: 77 - DNA, 24 bp, Artificial Sequence
  Synthetic Construct
ttgaccagttccgagcaaatggta

SEQ ID NO: 78 - DNA, 24 bp, Artificial Sequence
  Synthetic Construct
gacagtatcgatgttccagcagct

SEQ ID NO: 79 - DNA, 30 bp, Artificial Sequence
  Synthetic Construct
gagacgttttggatacatgtgaaagaaggc

SEQ ID NO: 80 - DNA, 26 bp, Artificial Sequence
  Synthetic Construct
cgatggttcacgggattctgcaattc

SEQ ID NO: 81 - DNA, 27 bp, Artificial Sequence
  Synthetic Construct
cgttttccaaatctgtaacagactggg

SEQ ID NO: 82 - DNA, 25 bp, Artificial Sequence
  Synthetic Construct
aggacgttgataggttggatgtgga

SEQ ID NO: 83 - DNA, 31 bp, Artificial Sequence
  Synthetic Construct
ggtagctatgtagggaagggataaacgctga

SEQ ID NO: 84 - DNA, 28 bp, Artificial Sequence
  Synthetic Construct
gcgctaaggagcttaacttctgtgttcg

SEQ ID NO: 85 - DNA, 22 bp, Artificial Sequence
  Synthetic Construct
gacggttgtcccggtttaagca

SEQ ID NO: 86 - DNA, 21 bp, Artificial Sequence
  Synthetic Construct
gctggtatcttcgactggtct

SEQ ID NO: 87 - DNA, 22 bp, Artificial Sequence
  Synthetic Construct
aggctgggtgtgtaagcgttgt

SEQ ID NO: 88 - DNA, 25 bp, Artificial Sequence
  Synthetic Construct
caagcaattcggttggatatccgtt

SEQ ID NO: 89 - DNA, 35 bp, Artificial Sequence
  Synthetic Construct
  misc_feature (16)..(16): n is 2,6-Diaminopurine
ggtaatgaattacctntctctgctggtttcttctt

SEQ ID NO: 90 - DNA, 27 bp, Artificial Sequence
  Synthetic Construct
  misc_feature (13)..(13): n is 2,6-Diaminopurine
accagcatcttcngcatcttctgtaaa

SEQ ID NO: 91 - DNA, 34 bp, Artificial Sequence
  Synthetic Construct
  misc_feature (13)..(13): n is 2,6-Diaminopurine
gaagttatgtttnctattcgaatcgtggtccagt

SEQ ID NO: 92 - DNA, 27 bp, Artificial Sequence
  Synthetic Construct
gttgtaaagccatgatgctcgtaacca

SEQ ID NO: 93 - DNA, 20 bp, Artificial Sequence
  Synthetic Construct
ggcatgcctgtttgagcgtc

SEQ ID NO: 94 - DNA, 29 bp, Artificial Sequence
  Synthetic Construct
gcttattgatatgcttaagttcagcgggt

SEQ ID NO: 95 - DNA, 22 bp, Artificial Sequence
  Synthetic Construct
tgaggcttgactatacaacacc

SEQ ID NO: 96 - DNA, 29 bp, Artificial Sequence
  Synthetic Construct
ctaaaatgaacagataaagtaagattcaa

SEQ ID NO: 97 - DNA, 31 bp, Artificial Sequence
  Synthetic Construct
ttttttttttgaggcttgactatacaacacc

SEQ ID NO: 98 - DNA, 38 bp, Artificial Sequence
  Synthetic Construct
ctaaaatgaacagataaagtaagattcaattttttttt

SEQ ID NO: 99 - DNA, 29 bp, Artificial Sequence
  Synthetic Construct
aaaacttatatgacttcaaatccagtttt

SEQ ID NO: 100 - DNA, 27 bp, Artificial Sequence
  Synthetic Construct
tttactcaataaaagataacaccacag

SEQ ID NO: 101 - DNA, 38 bp, Artificial Sequence
  Synthetic Construct
tttttttttaaaacttatatgacttcaaatccagtttt

SEQ ID NO: 102 - DNA, 37 bp, Artificial Sequence
  Synthetic Construct
tttactcaataaaagataacaccacagtttttttttt

SEQ ID NO: 103 - DNA, 24 bp, Artificial Sequence
  Synthetic Construct
tggataagtaaaagcaacttggtt

SEQ ID NO: 104 - DNA, 30 bp, Artificial Sequence
  Synthetic Construct
aatgaagattcaactcaataagaaacaaca

SEQ ID NO: 105 - DNA, 33 bp, Artificial Sequence
  Synthetic Construct
ttttttttttggataagtaaaagcaacttggtt

SEQ ID NO: 106 - DNA, 39 bp, Artificial Sequence
  Synthetic Construct
aatgaagattcaactcaataagaaacaacattttttttt

SEQ ID NO: 107 - DNA, 23 bp, Artificial Sequence
  Synthetic Construct
taccaaggcgcttgagagaactc

SEQ ID NO: 108 - DNA, 19 bp, Artificial Sequence
  Synthetic Construct
ctggtgtgtaggtgaagtc

SEQ ID NO: 109 - DNA, 32 bp, Artificial Sequence
  Synthetic Construct
ttttttttttaccaaggcgcttgagagaactc

SEQ ID NO: 110 - DNA, 28 bp, Artificial Sequence
  Synthetic Construct
ctggtgtgtaggtgaagtcttttttttt

SEQ ID NO: 111 - DNA, 23 bp, Artificial Sequence
  Synthetic Construct
gtgtgttgtagggtgaagtcgac

SEQ ID NO: 112 - DNA, 22 bp, Artificial Sequence
  Synthetic Construct
caccttgaaatcacatacctga

SEQ ID NO: 113 - DNA, 32 bp, Artificial Sequence
  Synthetic Construct
tttttttttgtgtgttgtagggtgaagtcgac

SEQ ID NO: 114 - DNA, 31 bp, Artificial Sequence
  Synthetic Construct
caccttgaaatcacatacctgattttttttt

SEQ ID NO: 115 - DNA, 23 bp, Artificial Sequence
  Synthetic Construct
ccatttgaagttgtttattatgc

SEQ ID NO: 116 - DNA, 27 bp, Artificial Sequence
  Synthetic Construct
gggaaatgattaattatgcattaaatc

SEQ ID NO: 117 - DNA, 32 bp, Artificial Sequence
  Synthetic Construct
tttttttttccatttgaagttgtttattatgc

SEQ ID NO: 118 - DNA, 36 bp, Artificial Sequence
  Synthetic Construct
gggaaatgattaattatgcattaaatcttttttttt

SEQ ID NO: 119 - DNA, 26 bp, Artificial Sequence
  Synthetic Construct
tttttcagatttaggattagttgatt

SEQ ID NO: 120 - DNA, 22 bp, Artificial Sequence
  Synthetic Construct
gatccgtattggttatatcatc

SEQ ID NO: 121 - DNA, 35 bp, Artificial Sequence
  Synthetic Construct
ttttttttttttttcagatttaggattagttgatt

SEQ ID NO: 122 - DNA, 31 bp, Artificial Sequence
  Synthetic Construct
gatccgtattggttatatcatcttttttttt

SEQ ID NO: 123 - DNA, 30 bp, Artificial Sequence
  Synthetic Construct
ttttttttttggaataatacgccgaccagc

SEQ ID NO: 124 - DNA, 34 bp, Artificial Sequence
  Synthetic Construct
aaggatctatttcagtatgatgcagttttttttt

SEQ ID NO: 125 - DNA, 200 bp, Artificial Sequence
  Synthetic Construct
tgccgaagcgttttccaaatctgtaacagactgggctgattgaatcttactttatctgttcattttagctagaggtataactaaatcaagttgtcttgcatatttaagaatcgattgatgctttatatacaactgcttgggtgttgtatagtcaagcctcacgagcaattagtattggtcagcttcacatatcactatgc

SEQ ID NO: 126 - DNA, 200 bp, Artificial Sequence
  Synthetic Construct
gcatgggaacaggtgtatccttctcgctatcgccaccacactgggtgttgtttcttattgagttgaatcttcattcactcaaaactggattgaagtttgaatcaaaataaccaagttgcttttacttatccattctttggttaagtcctcgaccgattagtattggtccgctccaactatcactagccttccacttccaa

SEQ ID NO: 127 - DNA, 200 bp, Artificial Sequence
  Synthetic Construct
gcatggttacaggtgtatccttctcgctatcgccaccacactgtggtgttatcttttattgagtaaattttgttcactcaaaactggatttgaagtcatataagtttttttccgagttcttttcttttaacctattggttaagtcctcgatcgattagtatcagtccgctccatacatcactgtacttccactcctgacc

SEQ ID NO: 128 - DNA, 200 bp, Artificial Sequence
  Synthetic Construct
cagctccatccgcagggacttcacctacacaccagcgtgccttctcccgaagttacggcaccattttgcctagttccttcacccgagttctctcaagcgccttggtattctctacctgaccacctgtgtcggtttggggtacgatttgatgttacctgatgcttagaggcttttcctggaagcagggcatttgttacttc

SEQ ID NO: 129 - DNA, 169 bp, Artificial Sequence
  Synthetic Construct
cgcttgggcttacgtctatccggattcaggtatgtgatttcaaggtgttttgcggttcatgcgaactttcggttcgtcgacttcaccttacaacacacaatcgtcagattgtttgggtgttatatggtcaagcctcacgggcaattagtactggttagctcaacgcctc

SEQ ID NO: 130 - DNA, 151 bp, Artificial Sequence
  Synthetic Construct
tttaccactaacaccatagaaattataacggtcaatgccatgatttaatgcataattaatcatttcccattgcactgcataacttccggcaaaatgacggaatgcatttgatgtaccaccagcataataaacaacttcaaatgggttgata

SEQ ID NO: 131 - DNA, 186 bp, Artificial Sequence
  Synthetic Construct
tgtgatttaaacaagtttactaaggcatcatttttctcgcgaccttcaaatggcacgatatctttatcatatagatgatataaccaatacggatctaatttaacatataaacattgatgttgctgtaaatatttatctaactcttttaaataataatcaactaatcctaaatctgaaaaatccatt

SEQ ID NO: 132 - DNA, 25 bp, Artificial Sequence
  Synthetic Construct
gcattaatcgacggtatggttgacc

SEQ ID NO: 133 - DNA, 26 bp, Artificial Sequence
  Synthetic Construct
cctgctgaaacaggttttcccacata

SEQ ID NO: 134 - DNA, 25 bp, Artificial Sequence
  Synthetic Construct
agtgatgatgagttgtttgccagtg

SEQ ID NO: 135 - DNA, 22 bp, Artificial Sequence
  Synthetic Construct
tgaattgtcgccgcgtgaccag

SEQ ID NO: 136 - DNA, 23 bp, Artificial Sequence
  Synthetic Construct
acccagcggtttgagggagaaac

SEQ ID NO: 137 - DNA, 30 bp, Artificial Sequence
  Synthetic Construct
aaagtttgaagatatacgtggtggacgtta

SEQ ID NO: 138 - DNA, 21 bp, Artificial Sequence
  Synthetic Construct
cgcacgcgcaagatggaaacg

SEQ ID NO: 139 - DNA, 23 bp, Artificial Sequence
  Synthetic Construct
aagttcagcgggtattcctacct

SEQ ID NO: 140 - DNA, 27 bp, Artificial Sequence
  Synthetic Construct
agctttttgttgtctcgcaacactcgc

SEQ ID NO: 141 - DNA, 26 bp, Artificial Sequence
  Synthetic Construct
ctaccaaacacaatgtgtttgagaag

SEQ ID NO: 142 - DNA, 31 bp, Artificial Sequence
  Synthetic Construct
cctgatttgaggtcaaacttaaagacgtctg

SEQ ID NO: 143 - DNA, 24 bp, Artificial Sequence
  Synthetic Construct
  misc_feature (22)..(22): n is nitroindole
agtcctacctgatttgaggtcnaa

SEQ ID NO: 144 - DNA, 21 bp, Artificial Sequence
  Synthetic Construct
  misc_feature (4)..(4): n is nitroindole
ccgngggtttgagggagaaat

SEQ ID NO: 145 - DNA, 33 bp, Artificial Sequence
  Synthetic Construct
aaagttatgaaataaattgtggtggccactagc

SEQ ID NO: 146 - DNA, 22 bp, Artificial Sequence
  Synthetic Construct
acccgggggtttgagggagaaa

SEQ ID NO: 147 - DNA, 24 bp, Artificial Sequence
  Synthetic Construct
agtcctacctgatttgaggtcgaa

SEQ ID NO: 148 - DNA, 21 bp, Artificial Sequence
  Synthetic Construct
ccgagggtttgagggagaaat

SEQ ID NO: 149 - DNA, 25 bp, Artificial Sequence
  Synthetic Construct
ggaagggatcaggtggttcactctt

SEQ ID NO: 150 - DNA, 29 bp, Artificial Sequence
  Synthetic Construct
aaaacttatgtgacttcaaatccagtttt

SEQ ID NO: 151 - DNA, 28 bp, Artificial Sequence
  Synthetic Construct
tttactcaataaaagataacaccacagt

SEQ ID NO: 152 - DNA, 38 bp, Artificial Sequence
  Synthetic Construct
tttttttttaaaacttatgtgacttcaaatccagtttt

SEQ ID NO: 153 - DNA, 23 bp, Artificial Sequence
  Synthetic Construct
tctgacgattgtgtgttgtaagg

SEQ ID NO: 154 - DNA, 20 bp, Artificial Sequence
  Synthetic Construct
ggatagacgtaagcccaagc

SEQ ID NO: 155 - DNA, 32 bp, Artificial Sequence
  Synthetic Construct
ttttttttttctgacgattgtgtgttgtaagg

SEQ ID NO: 156 - DNA, 29 bp, Artificial Sequence
  Synthetic Construct
ggatagacgtaagcccaagcttttttttt

SEQ ID NO: 157 - DNA, 198 bp, Artificial Sequence
  Synthetic Construct
gcatggttacaggtgtatccttctcgctatcgccaccacactgtggtgttatcttttattgagtaaattttgttcactcaaaactggatttgaagtcatataagtttttttccgagttcttttcttttaacctattggttaagtcctcgatcgattagtatcagtccgctccatacatcactgtacttccactcctga

SEQ ID NO: 158 - DNA, 19 bp, Artificial Sequence
  Synthetic Construct
ggcatgcctgtttgagcgt

SEQ ID NO: 159 - DNA, 20 bp, Artificial Sequence
  Synthetic Construct
gggcatgcctgtttgagcgt

SEQ ID NO: 160 - DNA, 27 bp, Artificial Sequence
  Synthetic Construct
ctacctgatttgaggcgacaacaaaac

SEQ ID NO: 161 - DNA, 24 bp, Artificial Sequence
  Synthetic Construct
ccgcgaagattggtgagaagacat

SEQ ID NO: 162 - DNA, 26 bp, Artificial Sequence
  Synthetic Construct
cctacctgatttgagggcgaaatgtc

SEQ ID NO: 163 - DNA, 19 bp, Artificial Sequence
  Synthetic Construct
ggagcaacgcctaaccggg

SEQ ID NO: 164 - DNA, 27 bp, Artificial Sequence
  Synthetic Construct
gtcctacctgatttgaggggaaaaagc

SEQ ID NO: 165 - DNA, 28 bp, Artificial Sequence
  Synthetic Construct
aacaaatccaccaacggtgagaagatat

SEQ ID NO: 166 - DNA, 19 bp, Artificial Sequence
  Synthetic Construct
cgtagacttcgctgcggat

SEQ ID NO: 167 - DNA, 20 bp, Artificial Sequence
  Synthetic Construct
gcgtagacttcgctgcggat

SEQ ID NO: 168 - DNA, 20 bp, Artificial Sequence
  Synthetic Construct
ctgggcggtgagaagaaatc

SEQ ID NO: 169 - DNA, 21 bp, Artificial Sequence
  Synthetic Construct
gcgtagacttcgctgctggaa

SEQ ID NO: 170 - DNA, 20 bp, Artificial Sequence
  Synthetic Construct
ccgtgcggtgagaagaaatc

SEQ ID NO: 171 - DNA, 28 bp, Artificial Sequence
  Synthetic Construct
tcctacctgatttgaggaaatagcatgg

SEQ ID NO: 172 - DNA, 22 bp, Artificial Sequence
  Synthetic Construct
atttagcggatgcaaaaccacc

SEQ ID NO: 173 - DNA, 20 bp, Artificial Sequence
  Synthetic Construct
ctcctacgggaggcagcagt

SEQ ID NO: 174 - DNA, 21 bp, Artificial Sequence
  Synthetic Construct
gtattaccgcggctgctggca

SEQ ID NO: 175 - DNA, 26 bp, Artificial Sequence
  Synthetic Construct
ctgacggagcaacgccgcgtgagtga

SEQ ID NO: 176 - DNA, 27 bp, Artificial Sequence
  Synthetic Construct
ctaaccagaaagccacggctaactacg

SEQ ID NO: 177 - DNA, 26 bp, Artificial Sequence
  Synthetic Construct
ctgatccagccatgccgcgtgtatga

SEQ ID NO: 178 - DNA, 27 bp, Artificial Sequence
  Synthetic Construct
ccgcagaagaagcaccggctaactccg

SEQ ID NO: 179 - DNA, 29 bp, Artificial Sequence
  Synthetic Construct
cacattcttttatcacgtaacgttggtgt

SEQ ID NO: 180 - DNA, 32 bp, Artificial Sequence
  Synthetic Construct
ccaggcattaccatttcagtaccttctggtaa

SEQ ID NO: 181 - DNA, 23 bp, Artificial Sequence
  Synthetic Construct
ccagttacgtcagtagtacggaa

SEQ ID NO: 182 - DNA, 23 bp, Artificial Sequence
  Synthetic Construct
tttgatttgaccacgttcaacac

SEQ ID NO: 183 - DNA, 27 bp, Artificial Sequence
  Synthetic Construct
agattgttcaaggacataatcaggtga

SEQ ID NO: 184 - DNA, 25 bp, Artificial Sequence
  Synthetic Construct
ggtaaatgaccttttctcgcccttc

SEQ ID NO: 185 - DNA, 21 bp, Artificial Sequence
  Synthetic Construct
ctcaggtgtgctggttattca

SEQ ID NO: 186 - DNA, 20 bp, Artificial Sequence
  Synthetic Construct
gccctgatcggattggagaa

SEQ ID NO: 187 - DNA, 20 bp, Artificial Sequence
  Synthetic Construct
cctcgggcaatggcgcaaac

SEQ ID NO: 188 - DNA, 22 bp, Artificial Sequence
  Synthetic Construct
atcgcgacggctttctgcctta

SEQ ID NO: 189 - DNA, 24 bp, Artificial Sequence
  Synthetic Construct
tattcatcaggaagtcgagccgga

SEQ ID NO: 190 - DNA, 24 bp, Artificial Sequence
  Synthetic Construct
aagatcaacacgggcaagccctct

SEQ ID NO: 191 - DNA, 26 bp, Artificial Sequence
  Synthetic Construct
atgtgggtccaattaattctgacgag

SEQ ID NO: 192 - DNA, 26 bp, Artificial Sequence
  Synthetic Construct
ctccagtttttggttgtaatgctgta

SEQ ID NO: 193 - DNA, 18 bp, Artificial Sequence
  Synthetic Construct
gggcgcagctggtgacat

SEQ ID NO: 194 - DNA, 20 bp, Artificial Sequence
  Synthetic Construct
aagatcgtgcgccgctgatt

SEQ ID NO: 195 - DNA, 22 bp, Artificial Sequence
  Synthetic Construct
gcagttataaccgttcccgcag

SEQ ID NO: 196 - DNA, 23 bp, Artificial Sequence
  Synthetic Construct
taacggccgcattgtactgaacg

SEQ ID NO: 197 - DNA, 22 bp, Artificial Sequence
  Synthetic Construct
aaaggctataaagatgatgcag

SEQ ID NO: 198 - DNA, 29 bp, Artificial Sequence
  Synthetic Construct
gagtatttataacaacatgaaaaatgatt

SEQ ID NO: 199 - DNA, 21 bp, Artificial Sequence
  Synthetic Construct
ggcttagaacgcctctatgat

SEQ ID NO: 200 - DNA, 31 bp, Artificial Sequence
  Synthetic Construct
agagtacaagaaagtatttataaacatatga

SEQ ID NO: 201 - DNA, 28 bp, Artificial Sequence
  Synthetic Construct
cactcgtaccgtctctgagggtgttgca

SEQ ID NO: 202 - DNA, 28 bp, Artificial Sequence
  Synthetic Construct
  misc_feature (16)..(16): n is g or t
cgtagattcacgaccnggaccagggcct

SEQ ID NO: 203 - DNA, 32 bp, Artificial Sequence
  Synthetic Construct
acaatggtgttattaaaagaggcgtgaatgtg

SEQ ID NO: 204 - DNA, 32 bp, Artificial Sequence
  Synthetic Construct
aaccgtagtctaaagcggctttaccagcaact

SEQ ID NO: 205 - DNA, 31 bp, Artificial Sequence
  Synthetic Construct
tatccgattattatgccagccatgtattcga

SEQ ID NO: 206 - DNA, 28 bp, Artificial Sequence
  Synthetic Construct
ccagtgggtgtagtgtgtcacgttgagc

SEQ ID NO: 207 - DNA, 32 bp, Artificial Sequence
  Synthetic Construct
actgctttgtatgcttctttgggaaggtattc

SEQ ID NO: 208 - DNA, 26 bp, Artificial Sequence
  Synthetic Construct
cagcttttcatgccgttggctatcag

SEQ ID NO: 209 - DNA, 20 bp, Artificial Sequence
  Synthetic Construct
cgctggcgcatgacgagaag

SEQ ID NO: 210 - DNA, 32 bp, Artificial Sequence
  Synthetic Construct
gcttgcggagatgccgtcataggtaccggctt

SEQ ID NO: 211 - DNA, 32 bp, Artificial Sequence
  Synthetic Construct
tagatgaagaaccatttccagtccatggaaac

SEQ ID NO: 212 - DNA, 19 bp, Artificial Sequence
  Synthetic Construct
cgttggcgatcagatgcag

SEQ ID NO: 213 - DNA, 33 bp, Artificial Sequence
  Synthetic Construct
gtttgtagtacatgaagcacctgagataacatc

SEQ ID NO: 214 - DNA, 31 bp, Artificial Sequence
  Synthetic Construct
cggaaaagaaatcaaagtaactgctaaaagc

SEQ ID NO: 215 - DNA, 26 bp, Artificial Sequence
  Synthetic Construct
accacgatatattagatggaagcgaa

SEQ ID NO: 216 - DNA, 27 bp, Artificial Sequence
  Synthetic Construct
gggagaattaggagtagacgtagtatt

SEQ ID NO: 217 - DNA, 21 bp, Artificial Sequence
  Synthetic Construct
tgatcccgttctggatggtga

SEQ ID NO: 218 - DNA, 27 bp, Artificial Sequence
  Synthetic Construct
ttccacataatccagatggaaccgact

SEQ ID NO: 219 - DNA, 18 bp, Artificial Sequence
  Synthetic Construct
gaatctgaatcaccgcgg

SEQ ID NO: 220 - DNA, 22 bp, Artificial Sequence
  Synthetic Construct
ccaggatagcgatgataatcac

SEQ ID NO: 221 - DNA, 27 bp, Artificial Sequence
  Synthetic Construct
tgagggaaaggcgaaaagaaccttggc

SEQ ID NO: 222 - DNA, 30 bp, Artificial Sequence
  Synthetic Construct
atgggtagatcaccgggtttcgggtctata

SEQ ID NO: 223 - DNA, 26 bp, Artificial Sequence
  Synthetic Construct
ttgtacgtacacggtttcaggttctt

SEQ ID NO: 224 - DNA, 23 bp, Artificial Sequence
  Synthetic Construct
cgcccggttaagactcggtttcc

SEQ ID NO: 225 - DNA, 27 bp, Artificial Sequence
  Synthetic Construct
tgtggtggcgatggcgagaaggataca

SEQ ID NO: 226 - DNA, 26 bp, Artificial Sequence
  Synthetic Construct
gaagcccacctcaagatgagatttcc

SEQ ID NO: 227 - DNA, 30 bp, Artificial Sequence
  Synthetic Construct
ctttatcaacgtaacggcattggtcaatac

SEQ ID NO: 228 - DNA, 31 bp, Artificial Sequence
  Synthetic Construct
cgcataggagggaaatggttattacttaaac

SEQ ID NO: 229 - DNA, 30 bp, Artificial Sequence
  Synthetic Construct
ggaaaacatctggcttgtgtgtttttaacc

SEQ ID NO: 230 - DNA, 24 bp, Artificial Sequence
  Synthetic Construct
ggttgtcatcattgatgttcccgc

SEQ ID NO: 231 - DNA, 21 bp, Artificial Sequence
  Synthetic Construct
gccgtcagtcagcagatgcag

SEQ ID NO: 232 - DNA, 22 bp, Artificial Sequence
  Synthetic Construct
gatcccctgctcggtcagtaat

SEQ ID NO: 233 - DNA, 21 bp, Artificial Sequence
  Synthetic Construct
cggcggactgggtacgacata

SEQ ID NO: 234 - DNA, 23 bp, Artificial Sequence
  Synthetic Construct
gcgaaaagctctttgcctaccag

SEQ ID NO: 235 - DNA, 23 bp, Artificial Sequence
  Synthetic Construct
tgatcccggtgttggcgatcgtg

SEQ ID NO: 236 - DNA, 27 bp, Artificial Sequence
  Synthetic Construct
ttcatgattccggtgacgatcagcgcc

SEQ ID NO: 237 - DNA, 26 bp, Artificial Sequence
  Synthetic Construct
tgcgcttcgataaacaggttcagcgg

SEQ ID NO: 238 - DNA, 21 bp, Artificial Sequence
  Synthetic Construct
ccacagcacctggcacaccag

SEQ ID NO: 239 - DNA, 22 bp, Artificial Sequence
  Synthetic Construct
ctgcatccgcgtgccaatatcg

SEQ ID NO: 240 - DNA, 26 bp, Artificial Sequence
  Synthetic Construct
agcatttccggtttgaagaaatgccc

SEQ ID NO: 241 - DNA, 18 bp, Artificial Sequence
  Synthetic Construct
cggcatcattcagcgggt

SEQ ID NO: 242 - DNA, 19 bp, Artificial Sequence
  Synthetic Construct
gggctttcaggcggctttc

SEQ ID NO: 243 - DNA, 27 bp, Artificial Sequence
  Synthetic Construct
tggtcaggtggtcagcgaagaggtcgg

SEQ ID NO: 244 - DNA, 22 bp, Artificial Sequence
  Synthetic Construct
cgtagtcggagtcgctgttctc

SEQ ID NO: 245 - DNA, 24 bp, Artificial Sequence
  Synthetic Construct
ggtgacgacgaccttgcgggcttt

SEQ ID NO: 246 - DNA, 18 bp, Artificial Sequence
  Synthetic Construct
tctgggccacgcggccgg

SEQ ID NO: 247 - DNA, 28 bp, Artificial Sequence
  Synthetic Construct
ccgcagaagaagcaccggctaactccgt

SEQ ID NO: 248 - DNA, 28 bp, Artificial Sequence
  Synthetic Construct
ctaaccagaaagccacggctaactacgt

SEQ ID NO: 249 - DNA, 27 bp, Artificial Sequence
  Synthetic Construct
tctgacggagcaacgccgcgtgagtga

SEQ ID NO: 250 - DNA, 29 bp, Artificial Sequence
  Synthetic Construct
cgcagaggtttatatgtcaattccagcta

SEQ ID NO: 251 - DNA, 26 bp, Artificial Sequence
  Synthetic Construct
tcttgataagcggcgaccagcttatt

SEQ ID NO: 252 - DNA, 29 bp, Artificial Sequence
  Synthetic Construct
caatattggatgcttgtaaagtttgaatt

SEQ ID NO: 253 - DNA, 24 bp, Artificial Sequence
  Synthetic Construct
ttcaacgccgggcgtacctaacgt

SEQ ID NO: 254 - DNA, 20 bp, Artificial Sequence
  Synthetic Construct
cgccgtarccgttgctgatg

SEQ ID NO: 255 - DNA, 28 bp, Artificial Sequence
  Synthetic Construct
cgatcaccacgtctaaacgtactgccga

SEQ ID NO: 256 - DNA, 25 bp, Artificial Sequence
  Synthetic Construct
cagtaccagttcgactttggtctgc

SEQ ID NO: 257 - DNA, 30 bp, Artificial Sequence
  Synthetic Construct
actggtggcctgaaatacgatgcgaacaac

SEQ ID NO: 258 - DNA, 25 bp, Artificial Sequence
  Synthetic Construct
cctgaagaagtccggtgccttcgta

SEQ ID NO: 259 - DNA, 27 bp, Artificial Sequence
  Synthetic Construct
agccataaagtccatgatgacgacaac

SEQ ID NO: 260 - DNA, 27 bp, Artificial Sequence
  Synthetic Construct
ccaaagtagaatggtactttcattgca

SEQ ID NO: 261 - DNA, 21 bp, Artificial Sequence
  Synthetic Construct
tcgcgcccaccagggttaaac

SEQ ID NO: 262 - DNA, 31 bp, Artificial Sequence
  Synthetic Construct
gctactggtcgtgttgaacgtggtcaaatca

SEQ ID NO: 263 - DNA, 27 bp, Artificial Sequence
  Synthetic Construct
acaacgcctgawcctacagtacgtcca

SEQ ID NO: 264 - DNA, 21 bp, Artificial Sequence
  Synthetic Construct
agatgtgtcatgtaaaccgat

SEQ ID NO: 265 - DNA, 25 bp, Artificial Sequence
  Synthetic Construct
atacttctgctttgaattcagtatg

SEQ ID NO: 266 - DNA, 27 bp, Artificial Sequence
  Synthetic Construct
gttgtgggtaccgatgatttcaacttc

SEQ ID NO: 267 - DNA, 36 bp, Artificial Sequence
  Synthetic Construct
aggtaaaaagtatacttcagctttgaatttagtgtg

SEQ ID NO: 268 - DNA, 26 bp, Artificial Sequence
  Synthetic Construct
tgcttgattcttcttgcataccgatg

SEQ ID NO: 269 - DNA, 22 bp, Artificial Sequence
  Synthetic Construct
aaaacttgaccacgttggacat

SEQ ID NO: 270 - DNA, 24 bp, Artificial Sequence
  Synthetic Construct
ctgatcatgctgtcgctgtggatc

SEQ ID NO: 271 - DNA, 21 bp, Artificial Sequence
  Synthetic Construct
ggtgatggtcagcaccgggaa

SEQ ID NO: 272 - DNA, 29 bp, Artificial Sequence
  Synthetic Construct
ctgtattcgatgcccgacagtggtgggaa

SEQ ID NO: 273 - DNA, 24 bp, Artificial Sequence
  Synthetic Construct
cggcatctgcatgagcttcatgct

SEQ ID NO: 274 - DNA, 33 bp, Artificial Sequence
  Synthetic Construct
ggcattggtccgtctgttgatgcaactacaagg

SEQ ID NO: 275 - DNA, 33 bp, Artificial Sequence
  Synthetic Construct
ggcattggaccgtcagttgaagcaactacaagg

SEQ ID NO: 276 - DNA, 31 bp, Artificial Sequence
  Synthetic Construct
tggcaaaagaaaaatacgatcgtagtaaacc

SEQ ID NO: 277 - DNA, 21 bp, Artificial Sequence
  Synthetic Construct
tcactggtgctgctcaaatgg

SEQ ID NO: 278 - DNA, 27 bp, Artificial Sequence
  Synthetic Construct
acgttaacattggtacaattggacacg

SEQ ID NO: 279 - DNA, 33 bp, Artificial Sequence
  Synthetic Construct
ccgtccgggtgatccaggtgatcatggtaaagg

SEQ ID NO: 280 - DNA, 30 bp, Artificial Sequence
  Synthetic Construct
gttggcagtaatgctgccgcgatctggcat

SEQ ID NO: 281 - DNA, 23 bp, Artificial Sequence
  Synthetic Construct
tggtcaatggcatattgagcaac

SEQ ID NO: 282 - DNA, 23 bp, Artificial Sequence
  Synthetic Construct
cttgcaaatggcgcatagaatcg

SEQ ID NO: 283 - DNA, 29 bp, Artificial Sequence
  Synthetic Construct
catattttgtgtagagtactgtgcaactt

SEQ ID NO: 284 - DNA, 25 bp, Artificial Sequence
  Synthetic Construct
attccaagtattagcaggtccataa

SEQ ID NO: 285 - DNA, 35 bp, Artificial Sequence
  Synthetic Construct
  misc_feature (9)..(9): n is inosine
cgttttccnatgtgcagtaccagtaaggttatggc

SEQ ID NO: 286 - DNA, 35 bp, Artificial Sequence
  Synthetic Construct
  misc_feature (9)..(9): n is inosine
cgttttgcnatgtgcagtaccagtaaggtgatggc

SEQ ID NO: 287 - DNA, 32 bp, Artificial Sequence
  Synthetic Construct
  misc_feature (27)..(27): n is inosine
ggtgaggtggtgtcgcgcgggtcgccngggat

SEQ ID NO: 288 - DNA, 32 bp, Artificial Sequence
  Synthetic Construct
  misc_feature (27)..(27): n is inosine
ggtgaggtggtgtctctcgggtcgccngggat

SEQ ID NO: 289 - DNA, 28 bp, Artificial Sequence
  Synthetic Construct
gctttcacttttcttcagcaccgcggcc

SEQ ID NO: 290 - DNA, 28 bp, Artificial Sequence
  Synthetic Construct
cgtttcactctgcttaagcaccgccgcg

SEQ ID NO: 291 - DNA, 27 bp, Artificial Sequence
  Synthetic Construct
cgtttcactttgcttgagcaccgccgt

SEQ ID NO: 292 - DNA, 27 bp, Artificial Sequence
  Synthetic Construct
ggcggtgtttaacgtcggctcggtacg

SEQ ID NO: 293 - DNA, 27 bp, Artificial Sequence
  Synthetic Construct
ggcggtattcagcgtaggttcagtgcg

SEQ ID NO: 294 - DNA, 27 bp, Artificial Sequence
  Synthetic Construct
cgcggtgttgagcgtcggctcagtacg

SEQ ID NO: 295 - DNA, 23 bp, Artificial Sequence
  Synthetic Construct
aaatgccctacagaccgaccaag

SEQ ID NO: 296 - DNA, 23 bp, Artificial Sequence
  Synthetic Construct
tgaacgccaccacaggcagtaaa

SEQ ID NO: 297 - DNA, 24 bp, Artificial Sequence
  Synthetic Construct
ccaatgatacgcatgataaacgct

SEQ ID NO: 298 - DNA, 21 bp, Artificial Sequence
  Synthetic Construct
caagacttgccacgatcaagc

SEQ ID NO: 299 - DNA, 22 bp, Artificial Sequence
  Synthetic Construct
ggtgaggcaatggcattgtcag

SEQ ID NO: 300 - DNA, 27 bp, Artificial Sequence
  Synthetic Construct
gtaatcggttatgtgcaaggtcatcgg

SEQ ID NO: 301 - DNA, 23 bp, Artificial Sequence
  Synthetic Construct
cttgcaagctcttgatatactgg

SEQ ID NO: 302 - DNA, 28 bp, Artificial Sequence
  Synthetic Construct
acttcttgtactggtgtaattttaaggg

SEQ ID NO: 303 - DNA, 23 bp, Artificial Sequence
  Synthetic Construct
atccctgcatgccgaacttcatg

SEQ ID NO: 304 - DNA, 21 bp, Artificial Sequence
  Synthetic Construct
gctctgcttcatctggcgctg

SEQ ID NO: 305 - DNA, 21 bp, Artificial Sequence
  Synthetic Construct
agacccgtgcattgaagagtt

SEQ ID NO: 306 - DNA, 21 bp, Artificial Sequence
  Synthetic Construct
ccagttctcttcggcgttagc

SEQ ID NO: 307 - DNA, 28 bp, Artificial Sequence
  Synthetic Construct
gcggcgattatagccggtcaaatgatga

SEQ ID NO: 308 - DNA, 19 bp, Artificial Sequence
  Synthetic Construct
tgcgtccgccatctgtgca

SEQ ID NO: 309 - DNA, 20 bp, Artificial Sequence
  Synthetic Construct
ccacgcagttccgaggcatt

SEQ ID NO: 310 - DNA, 21 bp, Artificial Sequence
  Synthetic Construct
acaaaggtcgtctctgtcagc

(19) United States
(12) Patent Application Publication
(10) Pub. No.: US 2022/0348986 A1
(43) Pub. Date: Nov. 3, 2022

(54) METHODS AND COMPOSITIONS FOR COMPREHENSIVE AND HIGH SENSITIVITY DETECTION OF PATHOGENS AND DRUG RESISTANCE MARKERS

(71) Applicant: T2 Biosystems, Inc., Lexington, MA (US)

(72) Inventors: Jessica Lee SNYDER, Woburn, MA (US); Joseph MARTURANO, Bedford, MA (US); Sandy ESTRADA, Fort Myers, FL (US); Robert Patrick SHIVERS, Watertown, MA (US); Roger SMITH, Westwood, MA (US); Brendan John MANNING, Arlington, MA (US); Thomas J. LOWERY, Jr., Sudbury, MA (US); Daniel GAMERO, Medford, MA (US); Mohamed Nabuan NAUFER, Malden, MA (US); Kelly N. BURGESS, North Reading, MA (US)

(21) Appl. No.: 17/620,230

(22) PCT Filed: Jun. 19, 2020
(86) PCT No.: PCT/US2020/038796

§ 371 Date: Dec. 17, 2021

Related U.S. Application Data
(60) Provisional application No. 62/863,538, filed on Jun. 19, 2019.

(51) Int. Cl. C12Q 1/689; C12Q 1/6895

(52) U.S. Cl. CPC C12Q 1/689; C12Q 1/6895; C12Q 1/6858

(57) ABSTRACT

Provided herein are methods of amplifying and detecting pathogens and drug resistance markers (e.g., antibiotic resistance genes) in complex samples (e.g., blood), as well as related panels and compositions (e.g., primers, probes, magnetic particles, systems, cartridges, and kits). The methods, panels, and compositions can be used for comprehensive detection of pathogens and drug resistance markers for patient identification, patient selection, optimization of therapies, and antimicrobial stewardship.

### FIELD OF THE INVENTION

The invention features methods and compositions for amplifying and detecting pathogen target nucleic acids, including drug resistance markers (e.g., antibiotic resistance genes), in complex samples, for example, blood (e.g., whole blood). The methods and compositions can be used, e.g., for patient identification, patient selection, optimization of therapies, and antimicrobial stewardship.

### BACKGROUND OF THE INVENTION

The current paradigm of in vitro diagnostic testing for patients suspected of bloodstream infections (e.g., bacteremia and fungemia), sepsis, and related conditions is laborious, insensitive, and requires multiple days. These bloodstream and tissue infections can be challenging to detect with existing methods due to the low titer level of the infectious pathogen in the sampled biofluid. Titer levels of microbial pathogens are typically less than 1 colony-forming unit (CFU)/mL to as high as 100 CFU/mL in these diseases. For example, blood culture is currently the reference standard for diagnosis of bloodstream infections. Blood culture may take from 1-5 days for sufficient growth to occur in a blood culture vial for a blood culture instrument to flag the culture as positive. Blood culture also generally has a low overall sensitivity, and at present, between 30% and 50% of patients have false negative results from blood culture and therefore do not receive adequate therapy. In addition, blood cultures have been shown to be contaminated with commensal species at rates >6% at some institutions, leading to false positive results. After blood culture positivity, an aliquot is typically characterized by microscopy, Gram staining, and further subculturing prior to species identification.

Drug resistance, such as antibiotic resistance in bacteria, is one of the biggest threats to global health, food security, and development. Antibiotic-resistant bacteria lead to increased mortality, longer hospital stays, and higher medical costs, and can affect patients of all ages and demographics. The proportion of drug-resistant organisms is expected to increase under the use of current diagnostic tools and empirical therapy. In one example, appropriate therapy for infection by carbapenem-resistant Enterobacteriaceae (CPEs) started within five days of infection is associated with lower mortality. However, current diagnostic methods typically require 1-5 days of blood culture before diagnostic tests (e.g., antimicrobial susceptibility testing or genomic testing) can be performed. This delay in detection can negatively impact patient outcomes, and inappropriate disease treatment preceding detection may also contribute to the spread of multidrug-resistant organisms.

Thus, there is an unmet need in the art for methods for the rapid and comprehensive detection of pathogen nucleic acids, including drug resistance markers (e.g., antibiotic resistance genes), directly from complex samples (e.g., whole blood).

### SUMMARY OF THE INVENTION

The invention features, inter alia, methods of amplifying and detecting pathogen target nucleic acids, including drug resistance markers (e.g., antibiotic resistance genes), from pathogens (e.g., drug-resistant pathogens) in complex samples (e.g., blood), as well as related panels and compositions (e.g., primers, probes, magnetic particles, systems, cartridges, and kits).

In one aspect, the invention features a method for detecting the presence of a pathogen in a biological sample, the method including: (a) amplifying in a biological sample or a fraction thereof one or more pathogen target nucleic acids in a multiplexed amplification reaction, wherein the multiplexed amplification reaction is configured to amplify a panel including at least 28 pathogen target nucleic acids, wherein the panel includes (i) one or more genus-level target nucleic acids, (ii) one or more Gram positive bacterial target nucleic acids, (iii) one or more Gram negative bacterial target nucleic acids, and (iv) one or more resistance gene target nucleic acids; and (b) detecting the one or more amplified pathogen target nucleic acids to determine whether the pathogen is present in the biological sample, wherein the method has a percent coverage of greater than or equal to 90% of pathogen species associated with infections of the sample.

In some embodiments, step (a) includes amplifying the one or more pathogen target nucleic acids in a lysate produced by lysing cells in the biological sample.

In another aspect, the invention features a method for detecting the presence of a pathogen in a biological sample, the method including: (a) providing a biological sample and optionally dividing the sample into one or more portions; (b) lysing pathogen cells in the biological sample or the one or more portions thereof to form one or more lysates; (c) amplifying, in the one or more lysates, one or more pathogen target nucleic acids in a multiplexed amplification reaction to form one or more amplified lysates, wherein the multiplexed amplification reaction is configured to amplify a panel including at least 28 pathogen target nucleic acids, wherein the panel includes (i) one or more genus-level target nucleic acids, (ii) one or more Gram positive bacterial target nucleic acids, (iii) one or more Gram negative bacterial target nucleic acids, and (iv) one or more resistance gene target nucleic acids; (d) preparing a plurality of assay samples by contacting the one or more amplified lysates with a plurality of populations of magnetic particles, wherein each population of magnetic particles has binding moieties characteristic of one or more of the pathogen target nucleic acids on its surface, the binding moieties operative to alter aggregation of the magnetic particles in the presence of an amplified pathogen target nucleic acid; (e) providing each assay sample in a detection tube within a device, the device including a support defining a well for holding the detection tube including the assay sample, and having an RF coil configured to detect a signal produced by exposing the mixture to a bias magnetic field created using one or more magnets and an RF pulse sequence; (f) exposing each assay sample to a bias magnetic field and an RF pulse sequence; (g) following step (f), measuring the signal produced by each assay sample; and (h) on the basis of the result of step (g), detecting whether one or more of the pathogens is present in the biological sample, wherein the method has a percent coverage of greater than or equal to 90% of pathogen species associated with infections of the sample.

In some embodiments of any of the preceding aspects, the lysate has at least about a 2:1, a 5:1, a 10:1, a 20:1, a 40:1, or a 60:1 higher concentration of cell debris relative to the biological sample.

In some embodiments, the cell debris is solid material.

In some embodiments of any of the preceding aspects, the biological sample has a volume of about 0.1 mL to about 5 mL. In some embodiments, the biological sample has a volume of about 2 mL.

In some embodiments of any of the preceding aspects, the biological sample is selected from the group consisting of blood, a bloody fluid, a tissue sample, bronchiolar lavage (BAL), urine, cerebrospinal fluid (CSF), synovial fluid (SF), and sputum. In some embodiments, the blood is whole blood, a crude blood lysate, serum, or plasma. In some embodiments, the whole blood is ethylenediaminetetraacetic acid (EDTA) whole blood, sodium citrate whole blood, sodium heparin whole blood, lithium heparin whole blood, or potassium oxylate/sodium fluoride whole blood. In some embodiments, the bloody fluid is wound exudate, wound aspirate, phlegm, or bile. In some embodiments, the tissue sample is a tissue sample from a transplant, a tissue biopsy (e.g., a skin biopsy, muscle biopsy, or lymph node biopsy), a homogenized tissue sample, or bone. In some embodiments, the biological sample is urine or BAL.

In some embodiments of any of the preceding aspects, the biological sample is a swab.

In another aspect, the invention features a method for detecting the presence of a pathogen in a whole blood sample, the method including: (a) amplifying, in one or more lysates produced from a whole blood sample, one or more pathogen target nucleic acids in a multiplexed amplification reaction, wherein the multiplexed amplification reaction is configured to amplify a panel including at least 28 pathogen target nucleic acids, wherein the panel includes (i) one or more genus-level target nucleic acids, (ii) one or more Gram positive bacterial target nucleic acids, (iii) one or more Gram negative bacterial target nucleic acids, and (iv) one or more resistance gene target nucleic acids, wherein each of the one or more lysates is produced by: (i) contacting the whole blood sample or a portion thereof with an erythrocyte lysis agent, thereby lysing red blood cells; (ii) centrifuging the product of step (i) to form a supernatant and a pellet; (iii) discarding some or all of the supernatant of step (ii) and resuspending the pellet to form an extract, optionally washing the pellet one or more times prior to resuspending the pellet; and (iv) lysing the remaining cells in the extract of step (iii) to form the lysate, the lysate containing both subject cell nucleic acid and pathogen nucleic acid; (b) detecting one or more amplified pathogen target nucleic acids in the one or more lysates, thereby detecting the presence of the pathogen in the sample, wherein the method has a percent coverage of greater than or equal to 90% of pathogen species associated with blood infections.

In some embodiments of any of the preceding aspects, the panel further includes (v) one or more pan-level target nucleic acids.

In some embodiments of any of the preceding aspects, the panel further includes (vi) one or more fungal target nucleic acids.

In some embodiments of any of the preceding aspects, the method has a percent coverage of greater than or equal to 95%, 96%, 97%, 98%, or 99% of pathogen species associated with infections of the sample. In some embodiments, the method has a percent coverage of greater than or equal to 99% of pathogen species associated with infections of the sample.

In some embodiments of any of the preceding aspects, the panel includes at least two subpanels.

In some embodiments, the panel includes at least four subpanels. In some embodiments, the panel includes five subpanels. In some embodiments, each subpanel includes at least six pathogen target nucleic acids. In some embodiments, each subpanel includes nine pathogen target nucleic acids. In some embodiments, each subpanel includes fourteen pathogen target nucleic acids. In some embodiments, each subpanel includes an internal control channel.

In some embodiments of any of the preceding aspects, the panel includes at least 36 pathogen target nucleic acids. In some embodiments, the panel includes at least 40 pathogen target nucleic acids. In some embodiments, the panel includes 45 pathogen target nucleic acids. In some embodiments, the 45 pathogen target nucleic acids are split between five subpanels.

In some embodiments of any of the preceding aspects, the one or more genus-level target nucleic acids are characteristic of a genus selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp., Staphylococcus spp., coagulase negative Staphylococcus spp., Streptococcus spp., Viridans group Streptococcus, Aspergillus spp., Candida spp., and Cryptococcus spp. In some embodiments, the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, or all twenty-two genus-level target nucleic acids selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen genus-level target nucleic acids selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments: (i) the genus-level target nucleic acid characteristic of Enterobacteriaceae is characteristic of Klebsiella spp., Enterobacter spp., Citrobacter spp., Serratia spp., Proteus spp., and/or Morganella spp.; (ii) the genus-level target nucleic acid characteristic of coagulase negative Staphylococcus spp. is characteristic of S. epidermidis, S. haemolyticus, S. lugdunensis, and/or S. hominis; (iii) the genus-level target nucleic acid characteristic of Viridans group Streptococcus is characteristic of S. anginosus, S. mitis, and/or S. oralis; and/or (iv) the genus-level target nucleic acid characteristic of anaerobes is characteristic of Clostridium spp. and/or Bacteroides spp. In some embodiments, the genus-level target nucleic acid is characteristic of Staphylococcus spp., and the Staphylococcus spp. target nucleic acid is amplified in the presence of a forward primer including the nucleotide sequence of CACATTCTTTTATCACGTAACGTTGGTGT (SEQ ID NO: 179) and a reverse primer including the nucleotide sequence of CCAGGCATTACCATTTCAGTACCTTCTGGTAA (SEQ ID NO: 180) to produce a Staphylococcus spp. amplicon. In some embodiments, a probe pair including a 5′ probe including the nucleotide sequence of CCAGTTACGTCAGTAGTACGGAA (SEQ ID NO: 181) and a 3′ probe including the nucleotide sequence of TTTGATTTGACCACGTTCAACAC (SEQ ID NO: 182) is used for detection of the Staphylococcus spp. amplicon. In some embodiments, the genus-level target nucleic acid is characteristic of Candida spp., and the Candida spp. target nucleic acid is amplified in the presence of a forward primer including a nucleotide sequence selected from the group consisting of GGCATGCCTGTTTGAGCGTC (SEQ ID NO: 93), GGCATGCCTGTTTGAGCGT (SEQ ID NO: 157), and GGGCATGCCTGTTTGAGCGT (SEQ ID NO: 159), and a reverse primer including the nucleotide sequence of GCTTATTGATATGCTTAAGTTCAGCGGGT (SEQ ID NO: 94) to produce a Candida spp. amplicon.

In some embodiments of any of the preceding aspects, the one or more Gram positive bacterial target nucleic acids are selected from the group consisting of E. faecium, E. faecalis, S. aureus, S. pneumoniae, S. pyogenes, and S. agalactiae. In some embodiments, the panel includes at least two, at least three, at least four, at least five, or all six Gram positive bacterial target nucleic acids selected from the group consisting of E. faecium, E. faecalis, S. aureus, S. pneumoniae, S. pyogenes, and S. agalactiae. In some embodiments, the one or more Gram positive bacterial target nucleic acids is amplified in the presence of a forward primer and a reverse primer set forth in Table 3. In some embodiments, the one or more Gram positive bacterial target nucleic acid amplicons is detected using a 5′ capture probe and a 3′ capture probe set forth in Table 4.

In some embodiments of any of the preceding aspects, the one or more Gram negative bacterial target nucleic acids are selected from the group consisting of A. baumannii, E. coli, H. influenzae, K. pneumoniae, P. aeruginosa, S. marcescens, P. mirabilis, and S. maltophilia. In some embodiments, the panel includes at least two, at least three, at least four, at least five, at least six, at least seven, or all eight Gram negative bacterial target nucleic acids selected from the group consisting of A. baumannii, E. coli, H. influenzae, K. pneumoniae, P. aeruginosa, S. marcescens, P. mirabilis, and S. maltophilia. In some embodiments, the one or more Gram negative bacterial target nucleic acids is amplified in the presence of a forward primer and a reverse primer set forth in Table 3. In some embodiments, the one or more Gram negative bacterial target nucleic acid amplicons is detected using a 5′ capture probe and a 3′ capture probe set forth in Table 4.

In some embodiments of any of the preceding aspects, the one or more resistance gene target nucleic acids are selected from the group consisting of mcr-1, mecA, mecC, mefA, mefE, vanA, vanB, ermA, ermB, KPC, NDM, VIM, IMP, OXA-23-like, OXA-48-like, SHV, CMY, DHA, CTX-M (e.g., CTX-M-2, CTX-M-8, CTX-M 14, and/or CTX-M 15), TEM, FKS, PDR1, and ERG11. In some embodiments, the one or more resistance gene target nucleic acids are selected from the group consisting mecA, mecC, mefA, mefE, vanA, vanB, ermA, ermB, KPC, NDM, VIM, IMP, OXA-23-like, OXA-48-like, SHV, CMY, DHA, CTX-M 14, CTX-M 15, TEM, FKS, PDR1, and ERG11. In some embodiments, the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, or all twenty-three resistance gene target nucleic acids selected from the group consisting of mecA, mecC, mefA, mefE, mcr-1, vanA, vanB, ermA, ermB, KPC, NDM, VIM, IMP, OXA-23-like, OXA-48-like, SHV, CMY, DHA, CTX-M, TEM, FKS, PDR1, and ERG11. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, or all twenty-three resistance gene target nucleic acids selected from the group consisting of mecA, mecC, mefA, mefE, vanA, vanB, ermA, ermB, KPC, NDM, VIM, IMP, OXA-23-like, OXA-48-like, SHV, CMY, DHA, CTX-M 14, CTX-M 15, TEM, FKS, PDR1, and ERG11. In some embodiments, the resistance target nucleic acid is characteristic of mecA and mecC; mefA and mefE; vanA and vanB; ermA and ermB; NDM, VIM, and IMP; CMY and DHA; or CTX-M. In some embodiments, the resistance target nucleic acid is characteristic of mecA and mecC; mefA and mefE; vanA and vanB; ermA and ermB; NDM, VIM, and IMP; CMY and DHA; or CTX-M 14 and CTX M 15.

In some embodiments of any of the preceding aspects, the resistance target nucleic acid characteristic of CTX-M is a universal CTX-M target nucleic acid. In some embodiments, the universal CTX-M target nucleic acid is characteristic of CTX-M-2, CTX-M-8, CTX-M-14, and CTX-M-15. In some embodiments, the universal CTX-M target nucleic acid is amplified in the presence of (i) a first degenerate forward primer comprising the nucleotide sequence of CGTTTTCCIATGTGCAGTACCAGTAAGGTTATGGC (SEQ ID NO: 285) and a second degenerate forward primer comprising the nucleotide sequence of CGTTTTGCIATGTGCAGTACCAGTAAGGTGATGGC (SEQ ID NO: 286) and (ii) a first degenerate reverse primer comprising the nucleotide sequence of GGTGAGGTGGTGTCGCGCGGGTCGCCIGGGAT (SEQ ID NO: 287) and a second degenerate reverse primer comprising the nucleotide sequence of GGTGAGGTGGTGTCTCTCGGGTCGCCIGGGAT (SEQ ID NO: 288). In some embodiments, a plurality of probes comprising (i) a plurality of 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a plurality of 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291) are used for detection of the universal CTX-M amplicon. In some embodiments, a first population of 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a second population of 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291), are used for detection of the universal CTX-M amplicon.

In some embodiments of any of the preceding aspects, the one or more resistance gene target nucleic acids is amplified in the presence of a forward primer and a reverse set forth in Table 10, Table 12, Table 14, Table 16, or Table 26. In some embodiments, the one or more resistance gene target nucleic acids is amplified in the presence of a forward primer and a reverse set forth in Table 10, Table 12, Table 14, or Table 16. In some embodiments, the one or more resistance gene target nucleic acid amplicons is detected using a 5′ capture probe and a 3′ capture probe set forth in Table 11, Table 13, Table 15, or Table 17, or Table 26. In some embodiments, the one or more resistance gene target nucleic acid amplicons is detected using a 5′ capture probe and a 3′ capture probe set forth in Table 11, Table 13, Table 15, or Table 17. In some embodiments, the resistance gene target nucleic acid is OXA-23-like, and the OXA-23-like target nucleic acid is amplified in the presence of a forward primer including the nucleotide sequence of AGATTGTTCAAGGACATAATCAGGTGA (SEQ ID NO: 183) and a reverse primer including the nucleotide sequence of GGTAAATGACCTTTTCTCGCCCTTC (SEQ ID NO: 184) to produce a OXA-23-like amplicon. In some embodiments, a probe pair including a 5′ probe including the nucleotide sequence of CTCAGGTGTGCTGGTTATTCA (SEQ ID NO: 185) and a 3′ probe including the nucleotide sequence of GCCCTGATCGGATTGGAGAA (SEQ ID NO: 186) is used for detection of the OXA-23-like amplicon.

In some embodiments of any of the preceding aspects, the one or more pan-level target nucleic acids are selected from the group consisting of Pan-Bacterial, Pan-Gram positive, Pan-Gram negative, and Pan-Fungal. In some embodiments, the panel includes at least two, at least three, or all four pan-level target nucleic acids selected from the group consisting of Pan-Bacterial, Pan-Gram positive, Pan-Gram negative, and Pan-Fungal. In some embodiments, the Pan-Bacterial target nucleic acid is amplified in the presence of a forward primer including the nucleotide sequence of CTCCTACGGGAGGCAGCAGT (SEQ ID NO: 173) and a reverse primer including the nucleotide sequence of GTATTACCGCGGCTGCTGGCA (SEQ ID NO: 174) to produce a Pan-Bacteria amplicon. In some embodiments, a probe pair including a 5′ probe including the nucleotide sequence of CTGACGGAGCAACGCCGCGTGAGTGA (SEQ ID NO: 175) and a 3′ probe including the nucleotide sequence of CTAACCAGAAAGCCACGGCTAACTACG (SEQ ID NO: 176) is used to detect the presence of Gram positive bacteria. In some embodiments, a probe pair including a 5′ probe including the nucleotide sequence of CTGATCCAGCCATGCCGCGTGTATGA (SEQ ID NO: 177) and a 3′ probe including the nucleotide sequence of CCGCAGAAGAAGCACCGGCTAACTCCG (SEQ ID NO: 178) is used to detect the presence of Gram negative bacteria.

In some embodiments of any of the preceding aspects, the one or more fungal target nucleic acids are selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the panel includes at least two, at least three, at least four, at least five, at least six, at least seven, at least eight, at least nine, at least ten, or all eleven fungal target nucleic acids selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the one or more fungal target nucleic acids is amplified in the presence of a forward primer and a reverse primer set forth in Table 7. In some embodiments, the one or more fungal target nucleic acid amplicons is detected using a 5′ capture probe and a 3′ capture probe set forth in Table 8 or Table 9.

In some embodiments of any of the preceding aspects, the panel is a panel shown in any one of Tables 20-24 or in Table 27. In some embodiments, the panel is a panel shown in any one of Tables 20-24. In some embodiments, the panel is shown in Table 27. In some embodiments, the panel includes: (i) a first subpanel including the following pathogen target nucleic acids: Pan Gram negative, E. coli, K. pneumoniae, Enterobacter spp., Enterobacter cloacae complex, Citrobacter spp., S. marcescens, P. mirabilis, Salmonella spp., and an internal control; (ii) a second subpanel including the following pathogen target nucleic acids: Acinetobacter spp., A. baumanii, P. aeruginosa, S. maltophilia, H. influenzae, KPC, NDM/VIM/IMP, OXA-48-like, CTX-M 14/15, and an internal control; (iii) a third subpanel including the following pathogen target nucleic acids: Pan Gram positive, Enterococcus spp., E. faecium, E. faecalis, Staphylococcus spp., S. aureus, coagulase negative Staphylococcus spp., mecA/C, vanA/B, and an internal control; (iv) a fourth subpanel including the following pathogen target nucleic acids: Streptococcus spp., S. pneumoniae, S. pyogenes, S. agalactiae, Viridans Group Streptococcus, Anaerobes, Corynebacterium spp., ermA/B, mefA/E, and an internal control; and (v) a fifth subpanel including the following pathogen target nucleic acids: Candida spp., C. albicans, C. tropicalis, C. parapsilosis, C. krusei, C. glabrata, C. auris, Aspergillus spp., Cryptococcus spp., and an internal control. In some embodiments, the panel comprises Pan Gram Positive, Pan Gram Negative, Staphylococcus aureus, Coagulase negative staphylococci, Enterococcus spp., Enterococcus faecium, Enterococcus faecalis, Streptococcus pneumoniae, Streptococcus agalactiae, Streptococcus pyogenes, Clostridium spp., Mycobacterium spp., Enterobacterales, Escherichia coli, Klebsiella pneumoniae, Klebsiella aerogenes, Enterobacter cloacae complex, Citrobacter spp., Serratia spp., Proteus spp., Acinetobacter baumannii, Bacteroides spp., Haemophilus influenzae, Pseudomonas aeruginosa, Stenotrophomonas maltophilia, mecA, mecC, vanA/B, mefA/E, KPC, NDM, VIM, IMP, OXA-48, OXA-23, OXA-24/40, CTX-M, AmpC, mcr-1, and strA/strB.

In some embodiments of any of the preceding aspects, amplifying is in the presence of whole blood proteins and non-target nucleic acids.

In some embodiments of any of the preceding aspects, lysing includes mechanical lysis or heat lysis. In some embodiments, the mechanical lysis is beadbeating or sonicating.

In some embodiments of any of the preceding aspects, the steps of the method are completed within 5 hours. In some embodiments, the steps of the method are completed within 4 hours. In some embodiments, the steps of the method are completed within 3 hours.

In some embodiments of any of the preceding aspects, the method detects a pathogen target nucleic acid of a pathogen present at a concentration of 10 cells/mL of biological sample or less. In some embodiments, the method detects a pathogen target nucleic acid of a pathogen present at a concentration of 3 cells/mL of biological sample. In some embodiments, the method detects a pathogen target nucleic acid of a pathogen present at a concentration of 1 cells/mL of biological sample.

In some embodiments of any of the preceding aspects, the method results in redundant detection of the pathogen at the pan level, genus level, species level, and/or resistance level.

In some embodiments of any of the preceding aspects, the method identifies the pathogen at the pan level.

In some embodiments of any of the preceding aspects, the method identifies the pathogen at the genus level.

In some embodiments of any of the preceding aspects, the method identifies the pathogen at the species level.

In some embodiments of any of the preceding aspects, the method identifies the pathogen at the resistance level.

In some embodiments of any of the preceding aspects, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR. In some embodiments, the PCR is symmetric PCR or asymmetric PCR.

In some embodiments of any of the preceding aspects, the detecting includes magnetic, sequencing, optical, fluorescent, mass, density, chromatographic, and/or electrochemical detection. In some embodiments, the detecting includes T2 magnetic resonance (T2MR).

In some embodiments of any of the preceding aspects, the detecting includes sequencing. In some embodiments, the sequencing includes massively parallel sequencing, Sanger sequencing, or single-molecule sequencing. In some embodiments, the massively parallel sequencing includes sequencing by synthesis or sequencing by ligation. In some embodiments, the massively parallel sequencing includes sequencing by synthesis. In some embodiments, the sequencing by synthesis includes ILLUMINA™ dye sequencing, ion semiconductor sequencing, or pyrosequencing. In some embodiments, the sequencing by synthesis includes ILLUMINA™ dye sequencing. In some embodiments, the sequencing by ligation includes sequencing by oligonucleotide ligation and detection (SOLiD™) sequencing or polony-based sequencing. In some embodiments, the single-molecule sequencing is nanopore sequencing, single-molecule real-time (SMRT™) sequencing, or Helicos™ sequencing.

In some embodiments of any of the preceding aspects: (i) each assay sample is contacted with 1×10⁶ to 1×10¹³ magnetic particles per milliliter of the biological sample; (ii) step (g) includes measuring the T2 relaxation response of the assay sample, and wherein increasing agglomeration in the assay sample produces an increase in the observed T2 relaxation time of the assay sample; (iii) the magnetic particles have a mean diameter of from 600 nm to 1200 nm; (iv) the magnetic particles have a T2 relaxivity per particle of from 1×10⁹ to 1×10¹² mM⁻¹s⁻¹; and/or (v) the magnetic particles are substantially monodisperse. In some embodiments, the magnetic particles have a mean diameter of from 650 nm to 950 nm. In some embodiments, the magnetic particles have a mean diameter of from 670 nm to 890 nm.

In another aspect, the invention features a method for identifying a patient infected with a pathogen, the method including: (a) providing a biological sample obtained from the subject; and (b) detecting the presence of a pathogen target nucleic acid in the biological sample according to any one of the methods described herein, wherein the presence of a pathogen target nucleic in the biological sample obtained from the subject identifies the subject as one who may be infected with the pathogen. In some embodiments, the method further includes selecting an optimized therapy for the patient based on the presence of the pathogen and/or the presence of one or more resistance genes.

In some embodiments of any of the preceding aspects, the method further includes administering the optimized therapy to the patient. In some embodiments, the method results in administration of an optimized therapy to the patient faster than standard of care. In some embodiments, the method results in de-escalation of empiric therapy within 3 to 5 hours.

In another aspect, provided herein is a primer comprising any one of the primer nucleotide sequences set forth herein, or a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or higher sequence identity to any one of the primer sequences disclosed herein.

In another aspect, provided herein is a probe comprising any one of the probe nucleotide sequences set forth herein, or a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or higher sequence identity to any one of the probe sequences disclosed herein.

In another aspect, provided herein is a magnetic particle conjugated to one or more of any of the probes disclosed herein.

In another aspect, provided herein is a removable cartridge comprising one or more of any of the primers, probes, and/or magnetic particles disclosed herein.

Other features and advantages of the invention will be apparent from the following detailed description, drawings, and the claims.

### BRIEF DESCRIPTION OF THE DRAWINGS

FIG. 1 is a graph showing percent coverage of species in epidemiology studies for an exemplary panel provided herein.

### DETAILED DESCRIPTION OF EMBODIMENTS OF THE INVENTION

The invention provides, inter alia, methods, panels, systems, cartridges, and kits for comprehensive amplification and/or detection of pathogen target nucleic acids, including drug resistance markers (e.g., antibiotic resistance genes), in complex biological or environmental samples containing cells, cell debris (e.g., blood), or non-specific nucleic acids (e.g., subject (e.g., host) cell DNA). The present invention is based, at least in part, on the development of panels that allow for amplification and/or detection of pathogen target nucleic acids that allow for high percent coverage (e.g., greater than 80%, greater than 85%, greater than 90%, greater than 91%, greater than 92%, greater than 93%, greater than 94%, greater than 95%, greater than 96%, greater than 97%, greater than 98%, or greater than 99% percent coverage) of pathogens that are associated with infections of complex biological samples, including blood.

In some embodiments, detection of pathogen nucleic acids, including drug resistance markers (e.g., antibiotic resistance genes) allows for rapid, accurate, and high sensitivity detection and identification of a pathogen (e.g., a drug-resistant microbial pathogen) present in a biological or environmental sample containing host cells, cell debris, and/or host cell nucleic acids (e.g., DNA), including but not limited to whole blood, processed whole blood (e.g., a crude whole blood lysate), serum, plasma, or other blood derivatives; bloody fluids such as wound exudate, phlegm, bile, and the like; bronchiolar lavage (BAL), urine, tissue samples (e.g., tissue biopsies); and sputum (e.g., purulent sputum and bloody sputum)), which may be used, for example, for diagnosis of a disease (e.g., sepsis, bloodstream infections (BSIs) (e.g., bacteremia, fungemia (e.g., Candidemia), and viremia), Lyme disease, septic shock, and diseases that may manifest with similar symptoms to diseases caused by or associated with drug resistant microbial pathogens, e.g., systemic inflammatory response syndrome (SIRS)).

In some embodiments, the methods, panels, systems, cartridges, and kits can involve T2MR detection of target nucleic acids. T2MR detection enables rapid and sensitive detection of drug resistance markers (e.g., antibiotic resistance genes) in complex samples. In some embodiments, the T2MR detection approaches employ magnetic particles. In some embodiments, the methods and systems employ an NMR unit, optionally one or more magnetic assisted agglomeration (MAA) units, optionally one or more incubation stations at different temperatures, optionally one or more vortexers, optionally one or more centrifuges, optionally a fluidic manipulation station, optionally a robotic system, and optionally one or more modular cartridges, as described in International Patent Application Publication No. WO 2012/054639, which is incorporated herein by reference in its entirety. In some embodiments, the methods of the invention are performed using a fully-automated system, e.g., which may contain a sequencing unit and, optionally, a NMR unit. T2MR approaches can be combined with sequencing. For example, in some embodiments, the T2MR detection can provide genus-level information that is used to direct or narrow sequencing in a sample.

In some embodiments, the methods, systems, cartridges, kits, and panels can involve sequencing of target nucleic acids. Any suitable sequencing approach can be used, e.g., massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and/or Sanger sequencing.

### Definitions

The terms “amplification” or “amplify” or derivatives thereof, as used herein, mean one or more methods known in the art for copying a target or template nucleic acid, thereby increasing the number of copies of a selected nucleic acid sequence. Amplification may be exponential or linear. A “target nucleic acid” refers to a nucleic acid or a portion thereof that is to be amplified, detected, and/or sequenced. A target or template nucleic acid may be any nucleic acid, including DNA or RNA. A target nucleic acid may be characteristic of a pathogen, also referred to as a “pathogen target nucleic acid.” A pathogen target nucleic acid may be characteristic of any of the pathogens described herein or known in the art. A pathogen target nucleic acid may include a drug resistance marker (e.g., an antibiotic resistance gene, e.g., an antibiotic resistance gene selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, or CTX-M 15), mefA, mefE, ermA, ermB, SHV, TEM, FKS, PDR1, and ERG11) or a portion thereof that is to be amplified, detected, and/or sequenced. The sequences amplified in this manner form an “amplified target nucleic acid,” “amplified region,” or “amplicon,” which are used interchangeably herein. Primers and/or probes can be readily designed to target a specific template nucleic acid sequence. Exemplary amplification approaches include but are not limited to polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, and ramification amplification (RAM).

The term “drug resistance” refers to the ability of a pathogen to resist one or more effects of a therapeutic agent. For example, “antimicrobial resistance” refers to the ability of a microbe (e.g., a bacterial or fungal pathogen) to resist one or more effects of an antimicrobial agent, and “antibiotic resistance” refers to the ability of a bacterium to resist one or more effects of an antibiotic agent. Drug-resistant pathogens can be more difficult to treat than drug-sensitive pathogens. Resistance can occur naturally in pathogens, or can arise via spontaneous mutation or by gene transfer between different species. A pathogen may be become resistant to a therapeutic agent that previously was able to treat an infection caused by the pathogen. In some embodiments, a drug-resistant pathogen is able to survive or proliferate upon exposure to a concentration of a therapeutic agent that would kill or slow proliferation of a drug-sensitive pathogen.

The terms “drug resistance gene,” a “resistance gene,” a “drug resistance target nucleic acid,” or a “resistance target nucleic acid” are used interchangeably herein and refer to a gene that confers or facilitates drug (e.g., antibiotic) resistance, or a portion thereof.

For example, an “antibiotic resistance gene,” or an “antibiotic resistance target nucleic acid” refers to a gene that confers or facilitates antibiotic resistance, or a portion thereof. Exemplary antibiotic (e.g., carbapenem) resistance genes include, but are not limited to, NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, or CTX-M 15), mefA, mefE, ermA, ermB, SHV, TEM, FKS, PDR1, and ERG11. Additional antibiotic resistance genes are described herein or are known in the art. In the literature, the enzymes encoded by these genes are typically spelled in capital letters, while the gene names are italicized. For example, the enzyme NDM is encoded by the bla_{NDM} gene. This convention generally holds for all of the beta lactamase genes (e.g., NDM, KPC, IMP, VIM, DHA, CMY, FOX, CTX-M, SHV, TEM, and OXA (e.g., OXA-23-like or OXA-48-like). In the present application, these terms are used interchangeably, and the capitalized shorthand terms, e.g., “NDM” may be used to refer to a nucleic acid for simplicity. Other resistance genes are typically italicized in the literature (mecA, mecC, vanA, vanB, mefA, mefE, ermA, ermB, FKS, PDR1, and ERG11), but in the present application, it is to be understood that italicized and non-italicized versions of these names are used interchangeably.

The terms “NDM” or “bla_{NDM}” refer to New Delhi metallo-beta-lactamase (e.g., NDM-1), as well as variants thereof, which may differ from NDM-1 by one or more amino acid substitutions, insertions, and/or deletions (including, e.g., NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, NDM-24, and NDM-27).

The terms “KPC” or “bla_{KPC}” refer to K. pneumoniae carbapenemase (e.g., KPC-2), as well as variants thereof, which may differ from KPC-2 by one or more amino acid substitutions, insertions, and/or deletions (including, e.g., KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, KPC-35, KPC-36, KPC-37, KPC-38, and KPC-39).

The terms “IMP” or “bla_{IMP}” refers to a metallo-beta-lactamase active on imipenem, including IMP-1, as well as variants thereof, which may differ from IMP-1 by one or more amino acid substitutions, insertions, and/or deletions (including, e.g., IMP-2, IMP-3, IMP-4, IMP-5, IMP-6, IMP-7, IMP-8, IMP-9, IMP-10, IMP-11, IMP-12, IMP-13, IMP-14, IMP-15, IMP-16, IMP-17, IMP-18, IMP-19, IMP-20, IMP-21, IMP-22, IMP-23, IMP-24, IMP-25, IMP-26, IMP-27, IMP-28, IMP-29, IMP-30, IMP-31, IMP-32, IMP-33, IMP-34, IMP-35, IMP-37, IMP-38, IMP-40, IMP-41, IMP-42, IMP-43, IMP-44, IMP-45, IMP-48, IMP-49, IMP-51, IMP-52, IMP-53, IMP-54, IMP-55, IMP-56, IMP-58, IMP-59, IMP-60, IMP-61, IMP-62, IMP-63, IMP-64, IMP-66, IMP-67, IMP-68, IMP-70, IMP-71, IMP-73, IMP-74, IMP-75, IMP-76, IMP-77, IMP-78, IMP-79, and IMP-80).

The terms “VIM” or “blaᵥᵢₘ” refers to Verona integron-encoded metallo-beta-lactamase, also referred to as VIM-1, as well as variants thereof, which may differ from VIM-1 by one or more amino acid substitutions, insertions, and/or deletions (including, e.g., VIM-2, VIM-3, VIM-4, VIM-5, VIM-6, VIM-7, VIM-8, VIM-9, VIM-10, VIM-11, VIM-12, VIM-13, VIM-14, VIM-15, VIM-16, VIM-17, VIM-18, VIM-19, VIM-20, VIM-23, VIM-24, VIM-25, VIM-26, VIM-27, VIM-28, VIM-29, VIM-30, VIM-31, VIM-32, VIM-33, VIM-34, VIM-35, VIM-36, VIM-37, VIM-38, VIM-39, VIM-40, VIM-41, VIM-42, VIM-43, VIM-44, VIM-45, VIM-46, VIM-47, VIM-49, VIM-50, VIM-51, VIM-52, VIM-53, VIM-54, VIM-56, VIM-57, VIM-58, VIM-59, VIM-60, VIM-61, and VIM-62).

The term “OXA” or “bla_{OXA}” refers to a group of carbapenem-hydroyzing class D beta lactamases originally named for their activity against oxacillin. Exemplary OXA beta lactamases include, without limitation, OXA-23-like and OXA-48-like beta lactamases.

The term “OXA-23-like” refers to a group of carbapenem-hydroyzing class D beta lactamases. This group encompasses OXA-23 (also referred to as bla_{OXA-}23) as well as OXA-23-like variants, which may differ from OXA-23 by one or more amino acid substitutions, insertions, and/or deletions (including, e.g., OXA-27, OXA-49, OXA-73, OXA-103, OXA-133, OXA-146, OXA-165, OXA-166, OXA-167, OXA-168, OXA-169, OXA-170, OXA-171, OXA-225, OXA-366, OXA-398, OXA-422, OXA-423, OXA-435, OXA-440, OXA-482, OXA-483, OXA-565, and OXA-657).

The term “OXA-48-like” refers to a group of carbapenem-hydroyzing class D beta lactamases. This group encompasses OXA-48 (also referred to as bla_{OXA-}48) as well as OXA-48-like variants, which may differ from OXA-48 by one or more amino acid substitutions, insertions, and/or deletions (including, e.g., OXA-162, OXA-163, OXA-181, OXA-199, OXA-204, OXA-232, OXA-244, OXA-245, OXA-247, OXA-252, OXA-370, OXA-405, OXA-416, OXA-438, OXA-439, OXA-484, OXA-505, OXA-514, OXA-515, OXA-517, OXA-519, OXA-538, OXA-546, OXA-547, OXA-566, OXA-567). A sequence alignment of OXA-48 and OXA-48-like variants is shown in FIG. 2 of Poirel et al. J. Antimicrob. Chemother. 67(7):1597-606, 2012.

The terms “DHA” or “bla_{DHA}” refer to plasmid-mediated Dhahran beta-lactamase, including DHA-1, as well as variants thereof, which may differ from DHA-1 by one or more amino acid substitutions, insertions, and/or deletions (including, e.g., DHA-2, DHA-3, DHA-4, DHA-5, DHA-6, DHA-7, DHA-10, DHA-12, DHA-13, DHA-14, DHA-15, DHA-16, DHA-17, DHA-18, DHA-19, DHA-20, DHA-21, DHA-22, DHA-23, DHA-24, DHA-25, DHA-26, DHA-27, and DHA-28).

The terms “CMY” or “bla_{CMY}” refers to a group of plasmid-mediated class C beta-lactamases that encode for resistance to antibiotics such as cephamycins, including CMY-2, as well as variants thereof, which may differ from CMY-2 by one or more amino acid substitutions, insertions, and/or deletions (including, e.g., CMY-4, CMY-5, CMY-6, CMY-7, CMY-12, CMY-13, CMY-14, CMY-15, CMY-16, CMY-17, CMY-18, CMY-20, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-36, CMY-37, CMY-38, CMY-39, CMY-40, CMY-41, CMY-42, CMY-43, CMY-44, CMY-45, CMY-46, CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-70, CMY-71, CMY-72, CMY-73, CMY-74, CMY-75, CMY-76, CMY-77, CMY-78, CMY-79, CMY-80, CMY-81, CMY-82, CMY-83, CMY-84, CMY-85, CMY-86, CMY-87, CMY-89, CMY-90, CMY-93, CMY-94, CMY-95, CMY-96, CMY-97, CMY-99, CMY-100, CMY-101, CMY-102, CMY-103, CMY-104, CMY-105, CMY-106, CMY-107, CMY-108, CMY-109, CMY-110, CMY-111, CMY-112, CMY-113, CMY-114, CMY-115, CMY-116, CMY-117, CMY-118, CMY-119, CMY-121, CMY-122, CMY-124, CMY-125, CMY-127, CMY-128, CMY-129, CMY-130, CMY-131, CMY-132, CMY-133, CMY-134, CMY-135, CMY-138, CMY-139, CMY-140, CMY-141, CMY-142, CMY-143, CMY-144, CMY-145, CMY-146, CMY-147, CMY-148, CMY-149, CMY-150, CMY-151, CMY-152, CMY-153, CMY-154, CMY-155, CMY-156, CMY-158, CMY-159, CMY-160, CMY-161, CMY-162, CMY-163, and BIL-1).

The term “mecA” refers to a gene that confers resistance to antibiotics such as methicillin and other beta-lactam antibiotics. Methicillin-resistant S. aureus (MRSA) is a commonly known carrier of the mecA gene. An exemplary mecA gene is provided in the NCBI AMR database under accession number NG_047937.1.

The term “mecC” refers to a gene that confers resistance to antibiotics such as methicillin and other beta-lactam antibiotics. mecC is a divergent homologue of mecA, and is also known as mecA_{LGA251}.

The term “vanA” refers to a class of antibiotic resistance genes conferring resistance to antibiotics such as vancomycin.

The term “vanB” refers to a class of antibiotic resistance genes conferring resistance to antibiotics such as vancomycin.

The terms “CTX-M” or “bla_{CTX-M}” refer to a class of extended spectrum beta-lactamases active on cefotaxime and first discovered in Munich. For example, in some embodiments the CTX-M belongs to the “CTX-M 2” group. In other embodiments, the CTX-M belongs to the “CTX-M 8” group. For example, in still further embodiments, the CTX-M belongs to the “CTX-M 14” group (also referred to as the CTX-M 9 group), which includes CTX-M-9, CTX-M-13, CTX-M-14, CTX-M-16, CTX-M-17, CTX-M-19, CTX-M-21, CTX-M-24, CTX-M-27, CTX-M-46, CTX-M-47, CTX-M-48, CTX-M-49, CTX-M-50, CTX-M-64, CTX-M-73, CTX-M-81, CTX-M-87, CTX-M-90, CTX-M-93, CTX-M-98, CTX-M-102, CTX-M-104, CTX-M-121, CTX-M-125, CTX-M-148, CTX-M-168, CTX-M-198, CTX-M-199, CTX-M-201, CTX-M-214, CTX-M-221, and CTX-M-223. In other embodiments, the CTX-M belongs to the “CTX-M 15” group (also referred to as the CTX-M 1 group), which includes CTX-M-1, CTX-M-3, CTX-M-10, CTX-M-12, CTX-M-15, CTX-M-22, CTX-M-23, CTX-M-28, CTX-M-29, CTX-M-30, CTX-M-32, CTX-M-33, CTX-M-36, CTX-M-42, CTX-M-53, CTX-M-54, CTX-M-55, CTX-M-61, CTX-M-66, CTX-M-69, CTX-M-71, CTX-M-72, CTX-M-80, CTX-M-82, CTX-M-101, CTX-M-114, CTX-M-116, CTX-M-117, CTX-M-144, CTX-M-166, CTX-M-170, CTX-M-178, CTX-M-179, CTX-M-180, CTX-M-181, CTX-M-182, CTX-M-186, CTX-M-187, CTX-M-188, CTX-M-189, CTX-M-190, CTX-M-197, CTX-M-206, CTX-M-207, and CTX-M-222. Other CTX-M variants are known in the art and may be detected using the approaches described herein.

The term “mefA” refers to a gene conferring resistance to antibiotics such as macrolides by encoding drug efflux pumps. The term encompasses subclasses of mefA, including mefA and mefE.

The term “erm” refers to a class of genes conferring resistance to antibiotics such as the macrolide erythromycin. The term encompasses, for example, ermA and ermB.

The terms “SHV” or “bla_{SHV}” refers to a class of beta-lactamases. The term encompasses, for example, SHV-1, as well as variants thereof, which may differ from SHV-1 by one or more amino acid substitutions, insertions, and/or deletions (including, e.g., SHV-1, SHV-1 b, SHV-2, SHV-2A, SHV-3, SHV-5, SHV-7, SHV-8, SHV-9, SHV-11, SHV-12, SHV-13, SHV-14, SHV-15, SHV-16, SHV-18, SHV-24, SHV-27, SHV-28, SHV-30, SHV-31, SHV-33, SHV-34, SHV-35, SHV-36, SHV-37, SHV-38, SHV-40, SHV-41, SHV-42, SHV-43, SHV-44, SHV-45, SHV-46, SHV-48, SHV-49, SHV-50, SHV-51, SHV-52, SHV-55, SHV-56, SHV-57, SHV-59, SHV-60, SHV-61, SHV-62, SHV-63, SHV-64, SHV-65, SHV-66, SHV-67, SHV-69, SHV-70, SHV-71, SHV-72, SHV-73, SHV-74, SHV-75, SHV-76, SHV-77, SHV-78, SHV-79, SHV-80, SHV-81, SHV-82, SHV-85, SHV-86, SHV-89, SHV-92, SHV-93, SHV-94, SHV-95, SHV-96, SHV-97, SHV-98, SHV-99, SHV-100, SHV-101, SHV-102, SHV-103, SHV-104, SHV-105, SHV-106, SHV-107, SHV-108, SHV-109, SHV-110, SHV-111, SHV-115, SHV-119, SHV-120, SHV-121, SHV-128, SHV-129, SHV-132, SHV-133, SHV-134, SHV-135, SHV-137, SHV-141, SHV-142, SHV-143, SHV-144, SHV-145, SHV-146, SHV-147, SHV-148, SHV-149, SHV-150, SHV-151, SHV-152, SHV-153, SHV-154, SHV-155, SHV-156, SHV-157, SHV-158, SHV-159, SHV-160, SHV-161, SHV-162, SHV-163, SHV-164, SHV-165, SHV-168, SHV-172, SHV-173, SHV-178, SHV-179, SHV-180, SHV-182, SHV-183, SHV-185, SHV-186, SHV-187, SHV-188, SHV-189, SHV-190, SHV-191, SHV-193, SHV-194, SHV-195, SHV-196, SHV-197, SHV-198, SHV-199, SHV-200, SHV-201, SHV-202, SHV-203, SHV-204, SHV-205, SHV-206, SHV-207, SHV-208, SHV-209, SHV-210, SHV-211, SHV-212, SHV-213, SHV-214, SHV-215, SHV-216, SHV-217, SHV-218, SHV-219, SHV-220, SHV-221, SHV-222, SHV-223, SHV-224, SHV-225, SHV-226, SHV-227, and SHV-228). For a review, see Liakoipolos et al. Front. Microbiol. 7:1374, 2016, which shows an alignment of SHV-type genes.

The terms “TEM” or “bla_{TEM}” refers to a class of beta-lactamases. The term encompasses, for example, TEM-1, as well as variants thereof, which may differ from TEM-1 by one or more amino acid substitutions, insertions, and/or deletions (including, e.g., TEM-2, TEM-3, TEM-4, TEM-6, TEM-8, TEM-9, TEM-10, TEM-11, TEM-12, TEM-15, TEM-16, TEM-17, TEM-19, TEM-20, TEM-21, TEM-22, TEM-24, TEM-26, TEM-28, TEM-29, TEM-30, TEM-32, TEM-33, TEM-34, TEM-35, TEM-36, TEM-40, TEM-43, TEM-45, TEM-47, TEM-48, TEM-49, TEM-52, TEM-53, TEM-54, TEM-55, TEM-56, TEM-57, TEM-60, TEM-63, TEM-67, TEM-68, TEM-70, TEM-71, TEM-72, TEM-76, TEM-77, TEM-78, TEM-79, TEM-80, TEM-81, TEM-82, TEM-83, TEM-84, TEM-85, TEM-86, TEM-87, TEM-88, TEM-90, TEM-91, TEM-92, TEM-93, TEM-94, TEM-95, TEM-96, TEM-97, TEM-98, TEM-99, TEM-101, TEM-102, TEM-104, TEM-105, TEM-106, TEM-107, TEM-108, TEM-109, TEM-110, TEM-111, TEM-112, TEM-113, TEM-114, TEM-115, TEM-116, TEM-120, TEM-121, TEM-122, TEM-123, TEM-124, TEM-125, TEM-126, TEM-127, TEM-128, TEM-129, TEM-130, TEM-131, TEM-132, TEM-133, TEM-134, TEM-135, TEM-136, TEM-137, TEM-138, TEM-139, TEM-141, TEM-142, TEM-143, TEM-144, TEM-145, TEM-146, TEM-147, TEM-148, TEM-149, TEM-150, TEM-151, TEM-152, TEM-153, TEM-154, TEM-155, TEM-156, TEM-157, TEM-158, TEM-159, TEM-160, TEM-162, TEM-163, TEM-164, TEM-166, TEM-167, TEM-168, TEM-169, TEM-171, TEM-176, TEM-177, TEM-178, TEM-181, TEM-182, TEM-183, TEM-184, TEM-185, TEM-186, TEM-187, TEM-188, TEM-189, TEM-190, TEM-191, TEM-193, TEM-194, TEM-195, TEM-196, TEM-197, TEM-198, TEM-201, TEM-205, TEM-206, TEM-207, TEM-208, TEM-209, TEM-210, TEM-211, TEM-212, TEM-213, TEM-214, TEM-215, TEM-216, TEM-217, TEM-219, TEM-220, TEM-224, TEM-225, TEM-226, TEM-227, TEM-229, TEM-230, TEM-231, TEM-233, TEM-234, TEM-236, and TEM-237).

As used herein, the terms “Enterobacteriaceae” and “Enterobacterales” are used interchangeably herein to refer to enteric Gram negative bacilli. The taxonomic family previously known as Enterobacteriaceae was divided into seven different families (Enterobacteriaceae, Erwiniaceae, Pectobacteriaceae, Yersiniaceae, and Budviciaceae) falling under the order “Enterobacterales” (see, e.g., Adeolu et al. Int. J. Syst. Evol. Microbiol. 66:5575-5599, 2016). The terms “Enterobacteriaceae” and “Enterobacterales” are used interchangeably to encompass bacteria included under either the previous taxonomy or the current taxonomy. In some embodiments, the Enterobacterales species are clinically relevant Enterobacterales spp.

As used herein, the terms “unit” or “units,” when used in reference to thermostable nucleic acid polymerases, refer to an amount of the thermostable nucleic acid polymerase (e.g., thermostable DNA polymerase). Typically a unit is defined as the amount of enzyme that will incorporate a particular amount of dNTPs (e.g., 10-20 nmol) into acid-insoluble material in 30-60 min at 65° C.-75° C. under particular assay conditions, although each manufacturer may define units differently. Unit definitions and assay conditions for commercially-available thermostable nucleic acid polymerases are known in the art. In some embodiments, one unit of thermostable nucleic acid polymerase (e.g., Taq DNA polymerase) may be the amount of enzyme that will incorporate 15 nmol of dNTP into acid-insoluble material in 30 min at 75° C. in an assay containing 1× ThermoPol® Reaction Buffer (New England Biosciences), 200 μM dNTPs including [³H]-dTTP, and 15 nM primed M13 DNA.

The term “sequencing” refers to any method for determining the nucleotide order of a nucleic acid (e.g., DNA), such as a target nucleic acid or an amplified target nucleic acid. Exemplary sequencing approaches include but are not limited to massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and Sanger sequencing. Massively parallel sequencing is also referred to in the art as next-generation or second-generation sequencing, and typically involves parallel sequencing of a large number (e.g., thousands, millions, or billions) of spatially-separated, clonally amplified templates or single nucleic acid molecules. Short reads are often used in massively parallel sequencing. See, e.g., Metzker, Nature Reviews Genetics 11:31-36, 2010. Long-read sequencing and/or single-molecule sequencing are sometimes referred to as third-generation sequencing. Hybrid approaches (e.g., massively parallel and single molecule approaches or massively parallel and long-read approaches) can also be used. It is to be understood that some approaches may fall into more than one category, for example, some approaches may be considered both second-generation and third-generation approaches, and some sources refer to both second and third generation sequencing as “next-generation” sequencing.

By “analyte” is meant a substance or a constituent of a sample to be analyzed. Exemplary analytes include one or more species of one or more of the following: a nucleic acid (e.g., DNA or RNA (e.g., mRNA)), an oligonucleotide, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, blood urea nitrogen (BUN), magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a pathogen), a cytoplasmic marker (e.g., CD4/CD8 or CD4/viral load), a therapeutic agent, a metabolite of a therapeutic agent, a marker for the detection of a weapon (e.g., a chemical or biological weapon), an organism, a pathogen, a pathogen byproduct, a parasite (e.g., a protozoan or a helminth), a protist, a fungus (e.g., yeast (e.g., a Candida species (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis)) or mold), a bacterium (e.g., Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakit), Enterobacterales spp., Enterobacteriaceae spp., Enterococcus spp. (e.g., Enterococcus faecium (including E. faecium with resistance marker vanA/B) and Enterococcus faecalis), Fusarium spp. (e.g., Fusarium solani), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus epidermidis, Staphylococcus haemolyticus, Staphylococcus lugdunensis, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkern), Ehrlichia spp. (including Ehrlichia chaffeensis, Ehrlichia ewingii, and Ehrlichia muris-like), Coxiella spp. (including Coxiella burnetit), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae)), an actinomycete, a cell (e.g., a whole cell, a tumor cell, a stem cell, a white blood cell, a T cell (e.g., displaying CD3, CD4, CD8, IL2R, CD35, or other surface markers), or another cell identified with one or more specific markers), a virus, a prion, a plant component, a plant by-product, algae, an algae by-product, plant growth hormone, an insecticide, a man-made toxin, an environmental toxin, an oil component, and components derived therefrom. In particular embodiments, the analyte is a nucleic acid (e.g., DNA or RNA (e.g., mRNA)), such as a target nucleic acid or an amplified target nucleic acid. In further particular embodiments, the analyte is a drug resistance marker (e.g., an antibiotic resistance gene, e.g., an antibiotic resistance gene selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, and/or CTX-M 15), mefA, mefE, ermA, ermB, SHV, TEM, FKS, PDR1, and ERG11) or a portion thereof.

A “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), urine, cerebrospinal fluid (CSF), synovial fluid (SF), breast milk, sweat, tears, saliva, semen, feces, vaginal fluid or tissue, sputum (e.g., purulent sputum and bloody sputum), nasopharyngeal aspirate or swab, lacrimal fluid, mucous, epithelial swab (e.g., a buccal swab, an axilla swab, a groin swab, an axilla/groin swab, or an ear swab), tissues (e.g., tissue biopsies (e.g., skin biopsies (e.g., from wounds, burns, or tick bites), muscle biopsies, or lymph node biopsies)), including tissue homogenates), organs, bones, teeth, or culture media (e.g., BHI, SABHI, SDA, LB, and the like), among others. In some embodiments, the biological sample is whole blood, which may contain an anticoagulant (e.g., EDTA, sodium citrate, sodium heparin, lithium heparin, and/or potassium oxylate/sodium fluoride). In several embodiments, the biological sample contains cells, cell debris, and/or nucleic acids (e.g., DNA) derived from the subject from which the sample was obtained. In particular embodiments, the subject is a host of a pathogen, and the biological sample obtained from the subject includes subject (host)-derived cells, cell debris, and nucleic acids (e.g., DNA), as well as one or more pathogen cells. The biological sample may be a swab sample, which may include a swab buffer diluent or swab transport medium. In some embodiments, the swab buffer diluent or swab transport medium is, without limitation, PBST, Amies Buffer, Amies Buffer+10% (v/v) 10× PBST, Cary Blair Media, or Liquid Stuart Swabs (which may include addition of 10% (v/v) 10× PBST). The biological sample may be a liquid sample.

As used herein, an “environmental sample” is a sample obtained from an environment, e.g., a surface swab sample, a sample from a building or a container, an air sample, a water sample, a soil sample, and the like. The environmental sample may contain any analyte described herein, e.g., a pathogen such as a bacterial (e.g., Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakit), Enterobacteriaceae spp., Enterococcus spp. (e.g., Enterococcus faecium (including E. faecium with resistance marker vanA/B) and Enterococcus faecalis), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetit), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae)), fungal (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and/or C. tropicalis)), protozoan, or viral organism or pathogen. In some embodiments, an environmental sample is from a hospital or other healthcare facility. In some embodiments, the environmental sample is a swab, which may include a swab buffer diluent or swab transport medium. In some embodiments, the swab buffer diluent or swab transport medium is, without limitation, PBST, Amies Buffer, Amies Buffer+10% (v/v) 10× PBST, Cary Blair Media, or Liquid Stuart Swabs (which may include addition of 10% (v/v) 10× PBST). The environmental sample may be a liquid sample.

A “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression. In some embodiments, the biomarker is a nucleic acid (e.g., DNA or RNA (e.g., mRNA)).

The term “cell debris” refers to any materials released from cells that have been lysed or otherwise died. Cell debris may include any material that is contained within a cell, e.g., nucleic acids, proteins (e.g., hemoglobin), lipids, glycolipids, small molecules, carbohydrates, heme compounds, membranes, and the like. In several embodiments, the cell debris is or includes solid material, such as solid material that can be concentrated with a liquid-solid separation method (e.g., centrifugation or filtration). In some examples, the cell debris is the solid material present after centrifugation (such as solid material produced by the sample processing procedure described in Examples 1-6 of U.S. Provisional Patent Application No. 62/729,375).

As used herein, the term “cells/mL” indicates the number of cells per milliliter of a biological or environmental sample. The number of cells may be determined using any suitable method, for example, hemocytometer, quantitative PCR, and/or automated cell counting. It is to be understood that in some embodiments, cells/mL may indicate the number of colony-forming units (CFU) per milliliter of a biological or environmental sample.

As used herein, the term “copies/mL” indicates the number of copies of a nucleic acid (e.g., a target nucleic acid characteristic of a pathogen or an antibiotic resistance gene, e.g., an antibiotic resistance gene selected from the group consisting of NDM, KPC, IMP, VIM, OXA (e.g., OXA-23-like or OXA-48-like), DHA, CMY, FOX, mecA, mecC, MCR, vanA, vanB, CTX-M (e.g., CTX-M-2, CTX-M-8, CTX-M 14, and/or CTX-M 15), MCR (e.g., mcr-1) mefA, mefE, ermA, ermB, SHV, TEM, FKS, PDR1, and ERG11) or a portion thereof per milliliter of a biological or environmental sample.

A “genus,” as used herein, refers to a grouping of organisms, including pathogens. In some embodiments, a genus may be a taxonomic classification, for instance, a taxonomic domain, a taxonomic kingdom, a taxonomic phylum, a taxonomic class, a taxonomic order, a taxonomic family, or a taxonomic genus. In other embodiments, a genus may be defined by any desired or suitable characteristics such as, for example, resistance to an antimicrobial agent or Gram staining (e.g., Gram negative or Gram positive). For example, the genus may be pan-Gram positive or pan-Gram negative. It is to be understood that, in some instances, a pathogen may belong to more than one genus.

A “genus-level” or “group-level” identification refers to identification of an analyte (e.g., a target nucleic acid) that provides information regarding a genus from which the analyte was obtained (e.g., a taxonomic classification, for instance, a taxonomic domain, a taxonomic kingdom, a taxonomic phylum, a taxonomic class, a taxonomic order, a taxonomic family, or a taxonomic genus). In some embodiments, a genus-level identification does not provide species-level identification.

The term “species,” as used herein, refers to a basic unit of biological classification as well as a taxonomic rank. A skilled artisan appreciates that a species may be defined based on a number of criteria, including, for example, DNA similarity, morphology, and ecological niche. The term encompasses any suitable species concept, including evolutionary species, phylogenetic species, typological species, genetic species, and reproductive species. The term also encompasses subspecies or strains.

A “species-level” identification refers to identification of an analyte (e.g., a target nucleic acid) that provides information regarding the species from which the analyte was obtained. With respect to target nucleic acids, in some embodiments, species-level identification provides information regarding nucleic acid variants (e.g., a single nucleotide polymorphism (SNP), an insertion/deletion (indel), a repetitive element, or a microsatellite repeat), which is also referred to herein as a “variant-level” identification. In some embodiments, a species-level or variant-level identification also provides a genus-level identification.

A “pathogen” means an agent causing disease or illness to its host, such as an organism or infectious particle, capable of producing a disease in another organism, and includes but is not limited to bacteria (e.g., Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakit), Enterobacteriaceae spp., Enterococcus spp. (e.g., Enterococcus faecium (including E. faecium with resistance marker vanA/B) and Enterococcus faecalis), Fusarium spp. (e.g., Fusarium solani), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetit), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae)), viruses, protozoa, prions, fungi (e.g., yeast (e.g., Candida species)), or pathogen by-products. “Pathogen by-products” are those biological substances arising from the pathogen that can be deleterious to the host or stimulate an excessive host immune response, for example pathogen nucleic acids, antigen(s), metabolic substances, enzymes, biological substances, or toxins (e.g., Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G). In some embodiments, the pathogen is a bacterial pathogen, e.g., a drug resistant bacterial pathogen, e.g., a bacterial pathogen that expresses one or more antibiotic resistance genes selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, and/or CTX-M 15), mefA, mefE, ermA, ermB, SHV, and TEM.

By “pathogen-associated analyte” is meant an analyte characteristic of the presence of a pathogen (e.g., a bacterium, fungus, or virus) in a sample. The pathogen-associated analyte can be a particular substance derived from a pathogen (e.g., a nucleic acid (e.g., DNA or RNA (e.g., mRNA)), a protein, lipid, polysaccharide, or any other material produced by a pathogen) or a mixture derived from a pathogen (e.g., whole cells, or whole viruses). In certain instances, the pathogen-associated analyte is selected to be characteristic of the genus, species, or specific strain of pathogen being detected. Alternatively, the pathogen-associated analyte is selected to ascertain a property of the pathogen, such as resistance to a particular therapy. In some embodiments, a pathogen-associated analyte may be a target nucleic acid that has been amplified. In other embodiments, a pathogen-associated analyte may be a host antibody or other immune system protein that is expressed in response to an infection by a pathogen (e.g., an IgM antibody, an IgA antibody, an IgG antibody, or a major histocompatibility complex (MHC) protein).

The term “percent coverage” refers to a percentage of pathogens (e.g., the percentage of isolates in an epidemiological study) that can be amplified and/or detected by a panel of the invention. For example, percent coverage can be calculated by determining the number of isolates in a study detected by a panel divided by the total number of isolates tested. In some embodiments, the percent coverage may be determined using any epidemiological study known in the art or described herein (e.g., in Table 25). In some embodiments, the study is Weinstein et al. Clin. Infect. Dis. 24:584-602, 1997; Reisner et al. J. Clin. Microbiol. 37(6):2024-2026, 1999; Karlowski et al. Annals of Clinical Microbiology and Antimicrobials 3:7, 2004; Kumar et al. Chest 136:1237-1248, 2009; or European Center for Disease Prevention and Control (ECDC) Surveillance Report, “Incidence and attributable mortality of healthcare-associated infections in intensive care units in Europe, 2008-2012”, 2012. In some embodiments, the data from two or more studies, e.g., two or more of the foregoing studies, can be averaged, and the resulting average can be used in calculation of percent coverage.

A “subject” is an animal, preferably a mammal (including, for example, rodents (e.g., mice or rats), farm animals (e.g., cows, sheep, horses, and donkeys), pets (e.g., cats and dogs), or primates (e.g., humans and non-human primates (e.g., monkeys, chimpanzees, and gorillas)). In particular embodiments, the subject is a human. A subject may be a patient (e.g., a patient having or suspected of having a disease associated with or caused by a pathogen). In some embodiments, a subject is a host of one or more pathogens.

By “pharmaceutical composition” is meant any composition that contains a therapeutically or biologically active agent (e.g., an antifungal agent) that is suitable for administration to a subject.

As used herein, by “administering” is meant a method of giving a dosage of a composition (e.g., a pharmaceutical composition) described herein (e.g., a composition comprising an antimicrobial agent (e.g., an antibiotic agent)) to a subject. The compositions utilized in the methods described herein can be administered by any suitable route, e.g., parenteral (for example, intravenous, intramuscular, intra-arterial, intracardiac, subcutaneous, or intraperitoneal), dermal, transdermal, ocular, inhalation, buccal, sublingual, perilingual, nasal, rectal, topical, and oral. The compositions utilized in the methods described herein can also be administered locally or systemically. The preferred method of administration can vary depending on various factors (e.g., the components of the composition being administered and the severity of the condition being treated).

As used herein, “linked” means attached or bound by covalent bonds, non-covalent bonds, and/or linked via Van der Waals forces, hydrogen bonds, and/or other intermolecular forces.

The term “magnetic particle” refers to particles including materials of high positive magnetic susceptibility such as paramagnetic compounds, superparamagnetic compounds, and magnetite, gamma ferric oxide, or metallic iron.

The terms “aggregation,” “agglomeration,” and “clustering” are used interchangeably in the context of the magnetic particles described herein and mean the binding of two or more magnetic particles to one another, for example, via a multivalent analyte, multimeric form of analyte, antibody, nucleic acid molecule, or other binding molecule or entity. In some instances, magnetic particle agglomeration is reversible. Such aggregation may lead to the formation of “aggregates,” which may include amplicons and magnetic particles bearing binding moieties.

As used herein, “nonspecific reversibility” refers to the colloidal stability and robustness of magnetic particles against non-specific aggregation in a liquid sample and can be determined by subjecting the particles to the intended assay conditions in the absence of a specific clustering moiety (i.e., an analyte or an agglomerator). For example, nonspecific reversibility can be determined by measuring the T₂ values of a solution of magnetic particles before and after incubation in a uniform magnetic field (defined as <5000 ppm) at 0.45T for 3 minutes at 37° C. Magnetic particles are deemed to have nonspecific reversibility if the difference in T₂ values before and after subjecting the magnetic particles to the intended assay conditions vary by less than 10% (e.g., vary by less than 9%, 8%, 6%, 4%, 3%, 2%, or 1%). If the difference is greater than 10%, then the particles exhibit irreversibility in the buffer, diluents, and matrix tested, and manipulation of particle and matrix properties (e.g., coating and buffer formulation) may be required to produce a system in which the particles have nonspecific reversibility. In another example, the test can be applied by measuring the T₂ values of a solution of magnetic particles before and after incubation in a gradient magnetic field 1 Gauss/mm-10000 Gauss/mm.

As used herein, the term “NMR relaxation rate” refers to a measuring any of the following in a sample T₁, T₂, T₁/T₂ hybrid, T₁ᵣₕₒ, T₂ᵣₕₒ, and T₂*. The systems and methods of the invention are designed to produce an NMR relaxation rate characteristic of whether an analyte is present in the liquid sample. In some instances the NMR relaxation rate is characteristic of the quantity of analyte present in the liquid sample.

As used herein, the term “T₁/T₂ hybrid” refers to any detection method that combines a T₁ and a T₂ measurement. For example, the value of a T₁/T₂ hybrid can be a composite signal obtained through the combination of, ratio, or difference between two or more different T₁ and T₂ measurements. The T₁/T₂ hybrid can be obtained, for example, by using a pulse sequence in which T₁ and T₂ are alternatively measured or acquired in an interleaved fashion. Additionally, the T₁/T₂ hybrid signal can be acquired with a pulse sequence that measures a relaxation rate that is comprised of both T₁ and T₂ relaxation rates or mechanisms.

By “pulse sequence” or “RF pulse sequence” is meant one or more radio frequency pulses to be applied to a sample and designed to measure, e.g., certain NMR relaxation rates, such as spin echo sequences. A pulse sequence may also include the acquisition of a signal following one or more pulses to minimize noise and improve accuracy in the resulting signal value.

As used herein, the term “signal” refers to an NMR relaxation rate, frequency shift, susceptibility measurement, diffusion measurement, or correlation measurements.

As used herein, reference to the “size” of a magnetic particle refers to the average diameter for a mixture of the magnetic particles as determined by microscopy, light scattering, or other methods.

As used herein, the term “substantially monodisperse” refers to a mixture of magnetic particles having a polydispersity in size distribution as determined by the shape of the distribution curve of particle size in light scattering measurements. The FWHM (full width half max) of the particle distribution curve less than 25% of the peak position is considered substantially monodisperse. In addition, only one peak should be observed in the light scattering experiments and the peak position should be within one standard deviation of a population of known monodisperse particles.

By “T₂ relaxivity per particle” is meant the average T₂ relaxivity per particle in a population of magnetic particles.

As used herein, “unfractionated” refers to an assay in which none of the components of the sample being tested are removed following the addition of magnetic particles to the sample and prior to the NMR relaxation measurement.

It is contemplated that units, methods, systems, cartridges, kits, panels, and processes of the claimed invention encompass variations and adaptations developed using information from the embodiments described herein. Throughout the description, where units, systems, cartridges, kits, or panels are described as having, including, or including specific components, or where processes and methods are described as having, including, or including specific steps, it is contemplated that, additionally, there are units, systems, cartridges, kits, or panels of the present invention that consist essentially of, or consist of, the recited components, and that there are processes and methods according to the present invention that consist essentially of, or consist of, the recited processing steps. It should be understood that the order of steps or order for performing certain actions is immaterial, unless otherwise specified, so long as the invention remains operable. Moreover, in many instances two or more steps or actions may be conducted simultaneously.

Methods for Comprehensive Amplification and/or Detection of Pathogen Target Nucleic Acids in Complex Samples

The invention provides methods of amplifying, detecting, and/or sequencing pathogen target nucleic acids (including resistance gene target nucleic acids) in complex biological or environmental samples containing cells, cell debris (e.g., blood), or non-specific nucleic acids (e.g., subject (e.g., host) cell DNA). In general, the methods provide coverage for a high percentage (e.g., greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, greater than 99%, or approximately 100%) of pathogens that are associated with infection in a given biological sample type (e.g., blood or other complex biological samples described herein).

For example, provided herein is a method for amplifying one or more target nucleic acids characteristic of a pathogen in a biological sample, the method including amplifying in a biological sample or a fraction thereof one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), wherein the method has a percent coverage of greater than or equal to 80% of pathogen species associated with infections of the sample (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with infections of the sample). The amplifying may be performed in a lysate produced by lysing cells in the biological sample. In some embodiments, the multiplexed amplification reaction is configured to amplify a panel including at least 28 pathogen target nucleic acids. In some embodiments, the method has a percent coverage of greater than or equal to 90% of pathogen species associated with infections of the sample.

In another example, provided herein is a method for detecting the presence of a pathogen in a biological sample, the method including: (a) amplifying in a biological sample or a fraction thereof one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids); and (b) detecting the one or more amplified pathogen target nucleic acids to determine whether the pathogen is present in the biological sample, wherein the method has a percent coverage of greater than or equal to 80% of pathogen species associated with infections of the sample (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with infections of the sample). In some embodiments, step (a) includes amplifying the one or more pathogen target nucleic acids in a lysate produced by lysing cells in the biological sample. In some embodiments, the multiplexed amplification reaction is configured to amplify a panel including at least 28 pathogen target nucleic acids. In some embodiments, the method has a percent coverage of greater than or equal to 90% of pathogen species associated with infections of the sample.

In a further example, provided herein is a method for amplifying one or more target nucleic acids characteristic of a pathogen in a biological sample, the method including: (a) providing a biological sample and optionally dividing the sample into one or more portions; (b) lysing pathogen cells in the biological sample or the one or more portions thereof to form one or more lysates; (c) amplifying, in the one or more lysates, one or more pathogen target nucleic acids in a multiplexed amplification reaction to form one or more amplified lysates, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), wherein the method has a percent coverage of greater than or equal to 80% of pathogen species associated with infections of the sample (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with infections of the sample). In some embodiments, the multiplexed amplification reaction is configured to amplify a panel including at least 28 pathogen target nucleic acids. In some embodiments, the method has a percent coverage of greater than or equal to 90% of pathogen species associated with infections of the sample.

In another example, provided herein is a method for detecting the presence of a pathogen in a biological sample, the method including: (a) providing a biological sample and optionally dividing the sample into one or more portions; (b) lysing pathogen cells in the biological sample or the one or more portions thereof to form one or more lysates; (c) amplifying, in the one or more lysates, one or more pathogen target nucleic acids in a multiplexed amplification reaction to form one or more amplified lysates, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids); (d) preparing a plurality of assay samples by contacting the one or more amplified lysates with a plurality of populations of magnetic particles, wherein each population of magnetic particles has binding moieties characteristic of one or more of the pathogen target nucleic acids on its surface, the binding moieties operative to alter aggregation of the magnetic particles in the presence of an amplified pathogen target nucleic acid; (e) providing each assay sample in a detection tube within a device, the device including a support defining a well for holding the detection tube including the assay sample, and having an RF coil configured to detect a signal produced by exposing the mixture to a bias magnetic field created using one or more magnets and an RF pulse sequence; (f) exposing each assay sample to a bias magnetic field and an RF pulse sequence; (g) following step (f), measuring the signal produced by each assay sample; and (h) on the basis of the result of step (g), detecting whether one or more of the pathogens is present in the biological sample, wherein the method has a percent coverage of greater than or equal to 80% of pathogen species associated with infections of the sample (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with infections of the sample). In some embodiments, the multiplexed amplification reaction is configured to amplify a panel including at least 28 pathogen target nucleic acids. In some embodiments, the method has a percent coverage of greater than or equal to 90% of pathogen species associated with infections of the sample.

In some embodiments of any of the methods described herein, the method (e.g., step (a) of the method) includes amplifying the target nucleic acid in a lysate produced by lysing cells in the environmental or biological sample. The lysate may contain a concentration of cells, cell debris, and/or non-target or subject-cell derived nucleic acids (e.g., DNA) relative to the original environmental or biological sample. As an example, 2 mL of a biological sample concentrated down to 0.1 mL in a lysate corresponds to a 20:1 higher concentration of debris compared to the original sample. If the lysate is diluted 1:1 for amplification, the amplification is performed in a lysate that represents a 10:1 concentration of the debris in the original sample. In some embodiments, the lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate is not diluted prior to amplification. In other embodiments, the lysate is diluted to produce a diluted lysate (e.g., for use in amplification), and the diluted lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, a bout 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate or the diluted lysate has about a 10:1 higher concentration of cell debris relative to the environmental or biological sample. In other embodiments, the lysate or the diluted lysate has about a 20:1 higher concentration of cell debris relative to the environmental or biological sample. In some embodiments, the cell debris is solid material (e.g., solid material that can be concentrated with a liquid-solid separation method (e.g., centrifugation or filtration). In some embodiments, the lysate or the amplified lysate solution is a super-saturated solution of cell debris (e.g., solid material).

For example, in some embodiments of any of the methods described herein, the lysate has at least about a 2:1, a 5:1, a 10:1, a 20:1, a 40:1, or a 60:1 higher concentration of cell debris relative to the biological sample. In some embodiments, the cell debris is solid material.

The biological sample may have any suitable volume. For example, in some embodiments, the biological sample has a volume of about 0.1 mL to about 20 mL (e.g., about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, about 10 mL, about 10.5 mL, about 11 mL, about 11.5 mL, about 12 mL, about 12.5 mL, about 13 mL, about 13.5 mL, about 14 mL, about 14.5 mL, about 15 mL, about 15.5 mL, about 16 mL, about 16.5 mL, about 17 mL, about 17.5 mL, about 18 mL, about 18.5 mL, about 19 mL, about 19.5 mL, or about 20 mL). In some embodiments, the biological sample has a volume of about 0.1 mL to about 5 mL. In some embodiments, the biological sample has a volume of about 0.1 mL to about 20 mL, about 0.1 mL to about 15 mL, about 0.1 mL to about 10 mL, about 0.1 mL to about 9 mL, about 0.1 mL to about 8 mL, about 0.1 mL to about 7 mL, about 0.1 mL to about 6 mL, about 0.1 mL to about 5 mL, about 0.1 mL to about 4 mL, about 0.1 mL to about 3 mL, about 0.1 mL to about 2 mL, about 0.1 mL to about 1 mL, about 1 mL to about 20 mL, about 1 mL to about 15 mL, about 1 mL to about 10 mL, about 1 mL to about 9 mL, about 1 mL to about 8 mL, about 1 mL to about 7 mL, about 1 mL to about 6 mL, about 1 mL to about 5 mL, about 1 mL to about 4 mL, about 1 mL to about 3 mL, about 1 mL to about 2 mL, about 2 mL to about 20 mL, about 2 mL to about 15 mL, about 2 mL to about 10 mL, about 2 mL to about 9 mL, about 2 mL to about 8 mL, about 2 mL to about 7 mL, about 2 mL to about 6 mL, about 2 mL to about 5 mL, about 2 mL to about 4 mL, about 2 mL to about 3 mL, about 3 mL to about 20 mL, about 3 mL to about 15 mL, about 3 mL to about 10 mL, about 3 mL to about 9 mL, about 3 mL to about 8 mL, about 3 mL to about 7 mL, about 3 mL to about 6 mL, about 3 mL to about 5 mL, about 3 mL to about 4 mL, about 4 mL to about 20 mL, about 4 mL to about 15 mL, about 4 mL to about 10 mL, about 4 mL to about 9 mL, about 4 mL to about 8 mL, about 4 mL to about 7 mL, about 4 mL to about 6 mL, about 4 mL to about 5 mL, about 5 mL to about 20 mL, about 5 mL to about 15 mL, about 5 mL to about 10 mL, about 5 mL to about 9 mL, about 5 mL to about 8 mL, about 5 mL to about 7 mL, about 5 mL to about 6 mL, about 6 mL to about 20 mL, about 6 mL to about 15 mL, about 6 mL to about 10 mL, about 6 mL to about 9 mL, about 6 mL to about 8 mL, about 6 mL to about 7 mL, about 7 mL to about 20 mL, about 7 mL to about 15 mL, about 7 mL to about 10 mL, about 7 mL to about 9 mL, about 7 mL to about 8 mL, about 8 mL to about 20 mL, about 8 mL to about 15 mL, about 8 mL to about 10 mL, about 8 mL to about 9 mL, about 9 mL to about 20 mL, about 9 mL to about 15 mL, about 9 mL to about 10 mL, about 10 mL to about 20 mL, about 10 mL to about 15 mL, or about 15 mL to about 20 mL. In some embodiments, the biological sample has a volume of about 2 mL.

Any suitable biological sample may be used in any of the methods described herein, including any biological sample described herein. In some embodiments, the biological sample is selected from the group consisting of blood, a bloody fluid, a tissue sample, bronchiolar lavage (BAL), urine, cerebrospinal fluid (CSF), synovial fluid (SF), and sputum. In some embodiments, the blood is whole blood, a crude blood lysate, serum, or plasma. In some embodiments, the whole blood is ethylenediaminetetraacetic acid (EDTA) whole blood, sodium citrate whole blood, sodium heparin whole blood, lithium heparin whole blood, or potassium oxylate/sodium fluoride whole blood. In some embodiments, the bloody fluid is wound exudate, wound aspirate, phlegm, or bile. In some embodiments, the tissue sample is a tissue sample from a transplant, a tissue biopsy (e.g., a skin biopsy, muscle biopsy, or lymph node biopsy), a homogenized tissue sample, or bone. In some embodiments, the biological sample is urine or BAL. In some embodiments, the biological sample is a swab.

In another example, provided herein is a method for amplifying one or more target nucleic acids characteristic of a pathogen in a whole blood sample, the method including amplifying, in one or more lysates produced from a whole blood sample, one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), wherein each of the one or more lysates is produced by: (i) contacting the whole blood sample or a portion thereof with an erythrocyte lysis agent, thereby lysing red blood cells; (ii) centrifuging the product of step (i) to form a supernatant and a pellet; (iii) discarding some or all of the supernatant of step (ii) and resuspending the pellet to form an extract, optionally washing the pellet one or more times prior to resuspending the pellet; and (iv) lysing the remaining cells in the extract of step (iii) to form the lysate, the lysate containing both subject cell nucleic acid and pathogen nucleic acid, wherein the method has a percent coverage of greater than or equal to 80% of pathogen species associated with blood infections (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with blood infections). In some embodiments, the multiplexed amplification reaction is configured to amplify a panel including at least 28 pathogen target nucleic acids. In some embodiments, the method has a percent coverage of greater than or equal to 90% of pathogen species associated with blood infections (e.g., 90%, 91%, 92%, 93%, 94%, 95%, 96%, 97%, 98%, 99%, or 100% of pathogen species associated with blood infections).

In yet another example, provided herein is a method for detecting the presence of a pathogen in a whole blood sample, the method including: (a) amplifying, in one or more lysates produced from a whole blood sample, one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), wherein each of the one or more lysates is produced by: (i) contacting the whole blood sample or a portion thereof with an erythrocyte lysis agent, thereby lysing red blood cells; (ii) centrifuging the product of step (i) to form a supernatant and a pellet; (iii) discarding some or all of the supernatant of step (ii) and resuspending the pellet to form an extract, optionally washing the pellet one or more times prior to resuspending the pellet; and (iv) lysing the remaining cells in the extract of step (iii) to form the lysate, the lysate containing both subject cell nucleic acid and pathogen nucleic acid; (b) detecting one or more amplified pathogen target nucleic acids in the one or more lysates, thereby detecting the presence of the pathogen in the sample, wherein the method has a percent coverage of greater than or equal to 80% of pathogen species associated with blood infections (e.g., greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% of pathogen species associated with blood infections). In some embodiments, the multiplexed amplification reaction is configured to amplify a panel including at least 28 pathogen target nucleic acids. In some embodiments, the method has a percent coverage of greater than or equal to 90% of pathogen species associated with infections of the sample (e.g., 90%, 91%, 92%, 93%, 94%, 95%, 96%, 97%, 98%, 99%, or 100% of pathogen species associated with blood infections).

In any of the methods described herein, the panel may further include (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids).

In any of the methods described herein, the panel may further include (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids).

Any of the methods described herein may have a percent coverage of greater than or equal to 90% (e.g., 90%, 91%, 92%, 93%, 94%, 95%, 96%, 97%, 98%, 99%, or 100%) of pathogen species associated with infections of the sample. For example, in some embodiments, the method has a percent coverage of greater than or equal to 95%, 96%, 97%, 98%, or 99% of pathogen species associated with infections of the sample. In some embodiments, the method has a percent coverage of greater than or equal to 99% of pathogen species associated with infections of the sample.

The panels described herein may be split across one or more subpanels. Any suitable number of subpanels may be used in any of the methods described herein. For example, in some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty subpanels. In some embodiments, the panel includes at least two subpanels. In some embodiments, the panel includes at least four subpanels. In some embodiments, the panel includes five subpanels.

Each subpanel may include any suitable number of pathogen target nucleic acids. For example, in some embodiments, the subpanel may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more pathogen target nucleic acids. For example, in some embodiments, the subpanel includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, or more pathogen target nucleic acids. In some embodiments, each subpanel includes at least 6 pathogen target nucleic acids. In some embodiments, each subpanel includes 9 pathogen target nucleic acids. In some embodiments, each subpanel includes 14 pathogen target nucleic acids. In some embodiments, each subpanel includes an internal control channel.

The panels described herein may include any suitable number of pathogen target nucleic acids.

For example, in some embodiments, the panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, 100, or more target nucleic acids. In some embodiments, the panel includes at least 28 pathogen target nucleic acids. In some embodiments, the panel includes at least 30 pathogen target nucleic acids. In some embodiments, the panel includes at least 32 pathogen target nucleic acids. In some embodiments, the panel includes at least 36 pathogen target nucleic acids. In some embodiments, the panel includes at least 36 pathogen target nucleic acids. In some embodiments, the panel includes at least 38 pathogen target nucleic acids. In some embodiments, the panel includes at least 40 pathogen target nucleic acids.

In some embodiments, the panel includes at least 42 pathogen target nucleic acids. In some embodiments, the panel includes at least 44 pathogen target nucleic acids. In some embodiments, the panel includes 45 pathogen target nucleic acids. In some embodiments, the 45 pathogen target nucleic acids are split between five subpanels.

For example, in some embodiments, the panel includes between 15 and 80, between 15 and 75, between 15 and 70, between 15 and 65, between 15 and 60, between 15 and 55, between 15 and 50, between 15 and 45, between 15 and 40, between 15 and 35, between 15 and 30, between 15 and 25, between 15 and 20, between 20 and 80, between 20 and 75, between 20 and 70, between 20 and 65, between 20 and 60, between 20 and 55, between 20 and 50, between 20 and 45, between 20 and 40, between 20 and 35, between 20 and 30, between 20 and 25, between 25 and 80, between 25 and 75, between 25 and 70, between 25 and 65, between 25 and 60, between 25 and 55, between 25 and 50, between 25 and 45, between 25 and 40, between 25 and 35, between 25 and 30, between 30 and 80, between 30 and 75, between 30 and 70, between 30 and 65, between 30 and 60, between 30 and 55, between 30 and 50, between 30 and 45, between 30 and 40, between 30 and 35, between 35 and 80, between 35 and 75, between 35 and 70, between 35 and 65, between 35 and 60, between 35 and 55, between 35 and 50, between 35 and 45, between 35 and 40, between 40 and 80, between 40 and 75, between 40 and 70, between 40 and 65, between 40 and 60, between 40 and 55, between 40 and 50, between 40 and 45, between 45 and 80, between 45 and 75, between 45 and 70, between 45 and 65, between 45 and 60, between 45 and 55, between 45 and 50, between 50 and 80, between 50 and 75, between 50 and 70, between 50 and 65, between 50 and 60, between 50 and 55, between 55 and 80, between 55 and 75, between 55 and 70, between 55 and 65, between 55 and 60, between 60 and 80, between 60 and 75, between 60 and 70, between 60 and 65, between 65 and 80, between 65 and 75, between 65 and 70, between 65 and 65, between 70 and 80, between 70 and 75, or between 75 and 80 pathogen target nucleic acids.

Any suitable genus-level target nucleic acids may be amplified, detected and/or sequenced in any of the methods described herein. For example, in some embodiments, the one or more genus-level target nucleic acids are characteristic of a genus selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp. In some embodiments, the one or more genus-level target nucleic acids are characteristic of a genus selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty genus-level target nucleic acids selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments, the genus-level target nucleic acid characteristic of Enterobacteriaceae is characteristic of Klebsiella spp., Enterobacter spp., Citrobacter spp., Serratia spp., Proteus spp., and/or Morganella spp.

In some embodiments, the genus-level target nucleic acid characteristic of coagulase negative Staphylococcus spp. is characteristic of S. epidermidis, S. haemolyticus, S. lugdunensis, and/or S. hominis. In some embodiments, the genus-level target nucleic acid characteristic of Viridans group Streptococcus is characteristic of S. anginosus, S. mitis, and/or S. oralis. In some embodiments, the genus-level target nucleic acid characteristic of anaerobes is characteristic of Clostridium spp. and/or Bacteroides spp.

In some embodiments of any of the methods described herein, the genus-level target nucleic acid may be characteristic of Staphylococcus spp., and the Staphylococcus spp. target nucleic acid is amplified in the presence of a forward primer including the nucleotide sequence of CACATTCTTTTATCACGTAACGTTGGTGT (SEQ ID NO: 179) and a reverse primer including the nucleotide sequence of CCAGGCATTACCATTTCAGTACCTTCTGGTAA (SEQ ID NO: 180) to produce a Staphylococcus spp. amplicon. In some embodiments, a probe pair including a 5′ probe including the nucleotide sequence of CCAGTTACGTCAGTAGTACGGAA (SEQ ID NO: 181) and a 3′ probe including the nucleotide sequence of TTTGATTTGACCACGTTCAACAC (SEQ ID NO: 182) is used for detection of the Staphylococcus spp. amplicon.

In some embodiments of any of the methods described herein, the genus-level target nucleic acid may be characteristic of Candida spp., and the Candida spp. target nucleic acid is amplified in the presence of a forward primer including a nucleotide sequence selected from the group consisting of GGCATGCCTGTTTGAGCGTC (SEQ ID NO: 93), GGCATGCCTGTTTGAGCGT (SEQ ID NO: 157), and GGGCATGCCTGTTTGAGCGT (SEQ ID NO: 159), and a reverse primer including the nucleotide sequence of GCTTATTGATATGCTTAAGTTCAGCGGGT (SEQ ID NO: 94) to produce a Candida spp. amplicon.

Any suitable Gram positive bacterial target nucleic acid may be amplified, detected and/or sequenced in any of the methods described herein. In some embodiments, the one or more Gram positive bacterial target nucleic acids are selected from the group consisting of E. faecium, E. faecalis, S. aureus, S. pneumoniae, S. pyogenes, and S. agalactiae. In some embodiments, the panel includes at least two, at least three, at least four, at least five, or all six Gram positive bacterial target nucleic acids selected from the group consisting of E. faecium, E. faecalis, S. aureus, S. pneumoniae, S. pyogenes, and S. agalactiae. In some embodiments, the one or more Gram positive bacterial target nucleic acids is amplified in the presence of a forward primer and a reverse primer set forth in Table 3 or in Table 26. In some embodiments, the one or more Gram positive bacterial target nucleic acids is amplified in the presence of a forward primer and a reverse primer set forth in Table 3. In some embodiments, the one or more Gram positive bacterial target nucleic acid amplicons is detected using a 5′ capture probe and a 3′ capture probe set forth in Table 4 or in Table 26. In some embodiments, the one or more Gram positive bacterial target nucleic acid amplicons is detected using a 5′ capture probe and a 3′ capture probe set forth in Table 4.

Any suitable Gram negative bacterial target nucleic acid may be amplified, detected and/or sequenced in any of the methods described herein. In some embodiments, the one or more Gram negative bacterial target nucleic acids are selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the panel includes at least two, at least three, at least four, at least five, at least six, at least seven, at least eight, at least nine, or all ten Gram negative bacterial target nucleic acids selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the one or more Gram negative bacterial target nucleic acids is amplified in the presence of a forward primer and a reverse primer set forth in Table 3 or in Table 26. In some embodiments, the one or more Gram negative bacterial target nucleic acid amplicons is detected using a 5′ capture probe and a 3′ capture probe set forth in Table 4 or in Table 26.

Any suitable resistance gene target nucleic acid may be amplified, detected and/or sequenced in any of the methods described herein. In some embodiments, the one or more resistance gene target nucleic acids are selected from the group consisting of mecA, mecC, mefA, mefE, MCR (e.g., mcr-1), vanA, vanB, ermA, ermB, KPC, NDM, VIM, IMP, OXA-23-like, OXA-48-like, SHV, CMY, DHA, CTX-M (e.g., CTX-M-2, CTX-M-8, CTX-M 14, and/or CTX-M 15), TEM, FKS, PDR1, and ERG11. In some embodiments, the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, or all twenty-three resistance gene target nucleic acids selected from the group consisting of mecA, mecC, mefA, mefE, mcr-1, vanA, vanB, ermA, ermB, KPC, NDM, VIM, IMP, OXA-23-like, OXA-48-like, SHV, CMY, DHA, CTX-M (e.g., CTX-M-2, CTX-M-8, CTX-M 14, and/or CTX-M 15), TEM, FKS, PDR1, and ERG11. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or all twenty-four resistance gene target nucleic acids selected from the group consisting of mecA, mecC, mefA, mefE, MCR, vanA, vanB, ermA, ermB, KPC, NDM, VIM, IMP, OXA-23-like, OXA-48-like, SHV, CMY, DHA, CTX-M 14, CTX-M 15, TEM, FKS, PDR1, and ERG11. In some embodiments, the resistance gene target nucleic acid is characteristic of mecA and mecC; mefA and mefE; vanA and vanB; ermA and ermB; NDM, VIM, and IMP; CMY and DHA; or CTX-M 14 and CTX M 15.

In some embodiments, the resistance target nucleic acid characteristic of CTX-M is a universal CTX-M target nucleic acid. In some embodiments, the universal CTX-M target nucleic acid is characteristic of CTX-M-2, CTX-M-8, CTX-M-14, and CTX-M-15. In some embodiments, the universal CTX-M target nucleic acid is amplified in the presence of (i) a first degenerate forward primer comprising the nucleotide sequence of CGTTTTCCIATGTGCAGTACCAGTAAGGTTATGGC (SEQ ID NO: 285) and a second degenerate forward primer comprising the nucleotide sequence of CGTTTTGCIATGTGCAGTACCAGTAAGGTGATGGC (SEQ ID NO: 286) and (ii) a first degenerate reverse primer comprising the nucleotide sequence of GGTGAGGTGGTGTCGCGCGGGTCGCCIGGGAT (SEQ ID NO: 287) and a second degenerate reverse primer comprising the nucleotide sequence of GGTGAGGTGGTGTCTCTCGGGTCGCCIGGGAT (SEQ ID NO: 288). In some embodiments, a plurality of probes comprising (i) a plurality of 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a plurality of 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291) are used for detection of the universal CTX-M amplicon. In some embodiments, a first population of 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a second population of 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291), are used for detection of the universal CTX-M amplicon.

The degenerate primers described above may be mixed together in the reaction allowing amplficiation of all CTX-M groups simultaneously. In some embodiments, all of the 3′ degenerate probes described above are mixed together and conjugated to a first population of particles and all of the 5′ degenerate probes are mixed and conjugated to a second population of particles. The 3′ and 5′ particle populations may be mixed, and this mixed population can be used to detect all members of the CTX-M groups.

In some embodiments, the one or more resistance gene target nucleic acids is amplified in the presence of a forward primer and a reverse set forth in Table 10, Table 12, Table 14, Table 16, or Table 26. In some embodiments, the one or more resistance gene target nucleic acids is amplified in the presence of a forward primer and a reverse set forth in Table 10, Table 12, Table 14, or Table 16. In some embodiments, the one or more resistance gene target nucleic acid amplicons is detected using a 5′ capture probe and a 3′ capture probe set forth in Table 11, Table 13, Table 15, Table 17, or Table 26. In some embodiments, the one or more resistance gene target nucleic acid amplicons is detected using a 5′ capture probe and a 3′ capture probe set forth in Table 11, Table 13, Table 15, or Table 17.

For example, in some embodiments of any of the methods described herein, the resistance gene target nucleic acid may be OXA-23-like, and the OXA-23-like target nucleic acid is amplified in the presence of a forward primer including the nucleotide sequence of AGATTGTTCAAGGACATAATCAGGTGA (SEQ ID NO: 183) and a reverse primer including the nucleotide sequence of GGTAAATGACCTTTTCTCGCCCTTC (SEQ ID NO: 184) to produce a OXA-23-like amplicon. In some embodiments, a probe pair including a 5′ probe including the nucleotide sequence of CTCAGGTGTGCTGGTTATTCA (SEQ ID NO: 185) and a 3′ probe including the nucleotide sequence of GCCCTGATCGGATTGGAGAA (SEQ ID NO: 186) is used for detection of the OXA-23-like amplicon.

Any suitable pan-level target nucleic acid may be amplified, detected and/or sequenced in any of the methods described herein. In some embodiments, the one or more pan-level target nucleic acids are selected from the group consisting of Pan-Bacterial, Pan-Gram positive, Pan-Gram negative, and Pan-Fungal. In some embodiments, the panel includes at least two, at least three, or all four pan-level target nucleic acids selected from the group consisting of Pan-Bacterial, Pan-Gram positive, Pan-Gram negative, and Pan-Fungal.

In any of the methods described herein, on some embodiments, the Pan-Bacterial target nucleic acid may be amplified in the presence of a forward primer including the nucleotide sequence of CTCCTACGGGAGGCAGCAGT (SEQ ID NO: 173) and a reverse primer including the nucleotide sequence of GTATTACCGCGGCTGCTGGCA (SEQ ID NO: 174) to produce a Pan-Bacteria amplicon. In some embodiments, a probe pair including a 5′ probe including the nucleotide sequence of CTGACGGAGCAACGCCGCGTGAGTGA (SEQ ID NO: 175) and a 3′ probe including the nucleotide sequence of CTAACCAGAAAGCCACGGCTAACTACG (SEQ ID NO: 176) is used to detect the presence of Gram positive bacteria. In some embodiments, a probe pair including a 5′ probe including the nucleotide sequence of CTGATCCAGCCATGCCGCGTGTATGA (SEQ ID NO: 177) and a 3′ probe including the nucleotide sequence of CCGCAGAAGAAGCACCGGCTAACTCCG (SEQ ID NO: 178) is used to detect the presence of Gram negative bacteria.

Any suitable fungal target nucleic acid may be amplified, detected and/or sequenced in any of the methods described herein. In some embodiments, the one or more fungal target nucleic acids are selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the panel includes at least two, at least three, at least four, at least five, at least six, at least seven, at least eight, at least nine, at least ten, or all eleven fungal target nucleic acids selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the one or more fungal target nucleic acids is amplified in the presence of a forward primer and a reverse primer set forth in Table 7. In some embodiments, the one or more fungal target nucleic acid amplicons is detected using a 5′ capture probe and a 3′ capture probe set forth in Table 8 or Table 9.

Any of the methods described herein may involve amplifying, detecting, and/or sequencing any of the panels described herein, e.g., in Example 1 or Example 2. In some embodiments, the panel is a panel shown in any one of Tables 20-24. In some embodiments, the panel is a panel shown in Table 27.

In some embodiments of any of the methods described herein, the panel includes: (i) a first subpanel including the following pathogen target nucleic acids: Pan Gram negative, E. coli, K. pneumoniae, Enterobacter spp., Enterobacter cloacae complex, Citrobacter spp., S. marcescens, P. mirabilis, Salmonella spp., and an internal control; (ii) a second subpanel including the following pathogen target nucleic acids: Acinetobacter spp., A. baumanii, P. aeruginosa, S. maltophilia, H. influenzae, KPC, NDM/VIM/IMP, OXA-48-like, CTX-M 14/15, and an internal control; (iii) a third subpanel including the following pathogen target nucleic acids: Pan Gram positive, Enterococcus spp., E. faecium, E. faecalis, Staphylococcus spp., S. aureus, coagulase negative Staphylococcus spp., mecA/C, vanA/B, and an internal control; (iv) a fourth subpanel including the following pathogen target nucleic acids: Streptococcus spp., S. pneumoniae, S. pyogenes, S. agalactiae, Viridans Group Streptococcus, Anaerobes, Corynebacterium spp., ermA/B, mefA/E, and an internal control; and (v) a fifth subpanel including the following pathogen target nucleic acids: Candida spp., C. albicans, C. tropicalis, C. parapsilosis, C. krusei, C. glabrata, C. auris, Aspergillus spp., Cryptococcus spp., and an internal control.

In some embodiments of any of the methods described herein, the panel includes Pan Gram Positive, Pan Gram Negative, Staphylococcus aureus, Coagulase negative staphylococci, Enterococcus spp., Enterococcus faecium, Enterococcus faecalis, Streptococcus pneumoniae, Streptococcus agalactiae, Streptococcus pyogenes, Clostridium spp., Mycobacterium spp., Enterobacterales, Escherichia coli, Klebsiella pneumoniae, Klebsiella aerogenes, Enterobacter cloacae complex, Citrobacter spp., Serratia spp., Proteus spp., Acinetobacter baumannii, Bacteroides spp., Haemophilus influenzae, Pseudomonas aeruginosa, Stenotrophomonas maltophilia, mecA, mecC, vanA/B, mefA/E, KPC, NDM, VIM, IMP, OXA-48, OXA-23, OXA-24/40, CTX-M, AmpC, mcr-1, and strA/strB.

In any of the methods described herein, the amplifying may be performed in the presence of whole blood proteins and non-target nucleic acids.

In any of the methods described herein, the lysing may include mechanical lysis or heat lysis. In some embodiments, the mechanical lysis is beadbeating or sonicating.

In some embodiments of any of the methods described herein, the steps of the method may be completed within 8 hours, e.g., within 8, 7, 6, 5, 4, 3, 2, or 1 hours. In some embodiments, the steps of the method are completed within 5 hours. In some embodiments, the steps of the method are completed within 4 hours. In some embodiments, the steps of the method are completed within 3 hours.

Any of the methods described herein may detect a pathogen target nucleic acid of a pathogen present at a concentration of 10 cells/mL of biological sample or less, e.g., 10 cells/mL or less, 9 cells/mL or less, 8 cells/mL or less, 7 cells/mL or less, 6 cells/mL or less, 5 cells/mL or less, 4 cells/mL or less, 3 cells/mL or less, 2 cells/mL or less, or 1 cell/mL or less. In some embodiments, the method detects a pathogen target nucleic acid of a pathogen present at a concentration of 3 cells/mL of biological sample.

In some embodiments, the method detects a pathogen target nucleic acid of a pathogen present at a concentration of 1 cell/mL of biological sample Any of the methods described herein may result in redundant detection of the pathogen at the pan level, genus level, species level, and/or resistance level.

Any of the methods described herein may identify the pathogen at the pan level.

Any of the methods described herein may identify the pathogen at the genus level.

Any of the methods described herein may identify the pathogen at the species level.

Any of the methods described herein may identify the pathogen at the resistance level.

In some embodiments of any of the methods described herein, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR. In some embodiments, the PCR is symmetric PCR or asymmetric PCR. In some embodiments, the PCR is asymmetric PCR.

In some embodiments of any of the methods described herein, the detecting includes magnetic, sequencing, optical, fluorescent, mass, density, chromatographic, and/or electrochemical detection.

In some embodiments of any of the methods described herein, the detecting includes T2 magnetic resonance (T2MR).

In some embodiments of any of the methods described herein, the detecting includes sequencing. In some embodiments, the sequencing includes massively parallel sequencing, Sanger sequencing, or single-molecule sequencing. In some embodiments, the massively parallel sequencing includes sequencing by synthesis or sequencing by ligation. In some embodiments, the massively parallel sequencing includes sequencing by synthesis. In some embodiments, the sequencing by synthesis includes ILLUMINA™ dye sequencing, ion semiconductor sequencing, or pyrosequencing. In some embodiments, the sequencing by synthesis includes ILLUMINA™ dye sequencing. In some embodiments, the sequencing by ligation includes sequencing by oligonucleotide ligation and detection (SOLiD™) sequencing or polony-based sequencing. In some embodiments, the single-molecule sequencing is nanopore sequencing, single-molecule real-time (SMRT™) sequencing, or Helicos™ sequencing.

An assay sample may be contacted with any suitable number of magnetic particles. For example, in some embodiments of any of the methods described herein, each assay sample is contacted with 1×10⁶ to 1×10¹³ magnetic particles per milliliter of the biological sample.

In some embodiments of any of the methods described herein, measuring the signal produced by each assay sample (e.g., in step (g) in certain methods described herein) includes measuring the T₂ relaxation response of the assay sample, and wherein increasing agglomeration in the assay sample produces an increase in the observed T₂ relaxation time of the assay sample.

Any of the magnetic particles described herein may be used in any of the methods. For example, in some embodiments of any of the methods described herein, the magnetic particles have a mean diameter of from 600 nm to 1200 nm. In some embodiments, the magnetic particles have a mean diameter of from 650 nm to 950 nm. In some embodiments, the magnetic particles have a mean diameter of from 670 nm to 890 nm.

The magnetic particles may have any suitable T₂ relaxivity per particle. For example, in some embodiments of any of the methods described herein, the magnetic particles have a T₂ relaxivity per particle of from 1×10⁹ to 1×10¹² mM⁻¹s⁻¹.

In some embodiments of any of the methods described herein, the magnetic particles are substantially monodisperse.

In some embodiments, the method comprises detecting any of the panels described herein, e.g., any panel set forth in any one of Tables 20-24 or Table 27. In some embodiments, the method comprises detecting a panel that includes one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 38, 39, or all 40), of Pan Gram Positive, Pan Gram Negative, Staphylococcus aureus, Coagulase negative staphylococci, Enterococcus spp., Enterococcus faecium, Enterococcus faecalis, Streptococcus pneumoniae, Streptococcus agalactiae, Streptococcus pyogenes, Clostridium spp., Mycobacterium spp., Enterobacterales, Escherichia coli, Klebsiella pneumoniae, Klebsiella aerogenes, Enterobacter cloacae complex, Citrobacter spp., Serratia spp., Proteus spp., Acinetobacter baumannii, Bacteroides spp., Haemophilus influenzae, Pseudomonas aeruginosa, Stenotrophomonas maltophilia, mecA, mecC, vanA/B, mefA/E, KPC, NDM, VIM, IMP, OXA-48, OXA-23, OXA-24/40, CTX-M, AmpC, mcr-1, and strA/strB.

In some embodiments, the method is capable of detecting greater than 90% (e.g., greater than 90%,

Any of the methods described herein may further include diagnosing the subject based on the detection of the target drug resistance (e.g., antibiotic resistance) nucleic acid, or the nucleotide sequence of the target drug resistance (e.g., antibiotic resistance) nucleic acid, wherein the presence or sequence of the target nucleic indicates that the subject is suffering from a disease associated with a drug-resistant pathogen. The method may further include administering to the subject a suitable therapy, e.g., a therapy tailored to the drug resistance profile of the pathogen based on the sequence of the target nucleic acid.

### Sample Preparation and Cell Lysis

The methods and systems of the invention may involve sample preparation and/or cell lysis. For example, an organism (e.g., a pathogen, including a a drug-resistant pathogen) present in a biological sample containing cells, cell debris, and/or nucleic acids (e.g., DNA or RNA (e.g., mRNA)), including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum may be lysed prior to amplification of a target nucleic acid. Suitable lysis methods for lysing cells (e.g., pathogen cells) in a biological sample include, for example, mechanical lysis (e.g., beadbeating and sonication), heat lysis, and alkaline lysis.

In some embodiments, the lysis method is beadbeating. In some embodiments, beadbeating may be performed by adding glass or silica beads (e.g., 0.5 mm glass or silica beads, 0.6 mm glass or silica beads, 0.7 mm glass or silica beads, 0.8 mm glass or silica beads, or 0.9 mm glass or silica beads) to a biological sample to form a mixture and agitating the mixture. As an example, the sample preparation and cell lysis (e.g., beadbeating) may be performed using any of the approaches and methods described in WO 2012/054639. Following lysis, the sample may include cell debris or nucleic acids derived from mammalia n host cells and/or from the pathogen cell(s) present in the sample.

In some embodiments, the methods of the invention may include preparing a tissue homogenate. Any suitable method or approach known in the art and/or described herein may be used, including but not limited to grinding (e.g., mortar and pestle grinding, cryogenic mortar and pestle grinding, or glass homogenizer), shearing (e.g., blender, rotor-stator, dounce homogenizer, or French press), beating (e.g., beadbeating), or sonication. In some embodiments, several approaches may be combined to prepare a tissue homogenate.

The methods of the invention may involve amplification, detection, and/or sequencing of one or more pathogen target nucleic acids (e.g., one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids), and/or (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids) in a whole blood sample. For example, the methods of the invention may involve amplification, detection, and/or sequencing of one or more antibiotic resistance genes (e.g., one or more antibiotic resistance genes selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, or CTX-M 15), mefA, mefE, ermA, ermB, SHV, TEM, FKS, PDR1, and ERG11) in a whole blood sample. In some embodiments, the methods may involve disruption of red blood cells (erythrocytes). In some embodiments, the disruption of the red blood cells can be carried out using an erythrocyte lysis agent (i.e., a lysis buffer, an isotonic lysis agent, or a nonionic detergent). Erythrocyte lysis buffers which can be used in the methods of the invention include, without limitation, isotonic solutions of ammonium chloride (optionally including carbonate buffer and/or EDTA), and hypotonic solutions. The basic mechanism of hemolysis using isotonic ammonium chloride is by diffusion of ammonia across red blood cell membranes. This influx of ammonium increases the intracellular concentration of hydroxyl ions, which in turn reacts with CO₂ to form hydrogen carbonate. Erythrocytes exchange excess hydrogen carbonate with chloride which is present in blood plasma via anion channels and subsequently increase in intracellular ammonium chloride concentrations. The resulting swelling of the cells eventually causes loss of membrane integrity.

Alternatively, the erythrocyte lysis agent can be an aqueous solution of nonionic detergents (e.g., nonyl phenoxypolyethoxylethanol (NP-40), 4-octylphenol polyethoxylate (TRITON™ X-100), BRIJ® 58, or related nonionic surfactants, and mixtures thereof). The erythrocyte lysis agent disrupts at least some of the red blood cells, allowing a large fraction of certain components of whole blood (e.g., certain whole blood proteins) to be separated (e.g., as supernatant following centrifugation) from the white blood cells or other cells (e.g., pathogen cells (e.g., bacterial cells and/or fungal cells)) present in the whole blood sample. Following erythrocyte lysis and centrifugation, the resulting pellet may be lysed, for example, as described above.

In some embodiments, the methods provided herein may include (a) providing a whole blood sample from a subject; (b) mixing the whole blood sample with an erythrocyte lysis agent solution to produce disrupted red blood cells; (c) following step (b), centrifuging the sample to form a supernatant and a pellet, discarding some or all of the supernatant, and resuspending the pellet to form an extract, (d) lysing cells of the extract (which may include white blood cells and/or pathogen cells) to form a lysate. In some embodiments, the method further comprises amplifying one or more pathogen nucleic acids (including drug resistance (e.g., antibiotic resistance) target nucleic acids) in the lysate. In some embodiments, the method further comprises sequencing one or more pathogen nucleic acids (including drug resistance (e.g., antibiotic resistance) target nucleic acids) in the lysate. In some embodiments, the sample of whole blood is from about 0.5 to about 10 mL of whole blood, for example, 0.5 mL, 1 mL, 2 mL, 3 mL, 4 mL, 5 mL, 6 mL, 7 mL, 8 mL, 9 mL, or 10 mL of whole blood. In some embodiments, the method may include washing the pellet (e.g., with a buffer such as TE buffer) prior to resuspending the pellet and optionally repeating step (c). In some embodiments, step (c) does not involve resuspending the pellet but instead includes adding a buffer solution to the pellet to form the extract. In some embodiments, the method may include 1, 2, 3, 4, 5, or more wash steps. In other embodiments, the method is performed without performing any wash step. In some embodiments, the amplifying is in the presence of whole blood proteins, non-target nucleic acids, or both. In some embodiments, the amplifying may be in the presence of from about 0.5 μg to about 200 μg (e.g., about 0.5 μg, 1 μg, 5 μg, 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110 μg, 120 μg, 130 μg, 140 μg, 150 μg, 160 μg, 170 μg, 180 μg, 190 μg, or 200 μg) of subject (i.e., host) DNA. In some embodiments, the amplifying may be in the presence of more than about 1 μg (e.g., more than about 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110 μg, 120 μg, 130 μg, 140 μg, 150 μg, 160 μg, 170 μg, 180 μg, 190 μg, or 200 μg) of subject (i.e., host) DNA. In some embodiments, at least a portion of the subject (i.e., host) DNA is from white blood cells of the subject. In some embodiments, the subject (i.e., host) DNA is from white blood cells of the subject.

### Amplification Approaches

In several embodiments, the methods and systems of the invention involve amplification of one or more nucleic acids. Amplification may be exponential or linear. A target or template nucleic acid may be any suitable nucleic acid(s) (e.g., one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids), and/or (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids).

In some embodiments, the target nucleic acid is DNA or RNA (e.g., mRNA). The sequences amplified in this manner form an amplified target nucleic acid (also referred to herein as an amplicon). Primers and probes can be readily designed by those skilled in the art to target a specific template nucleic acid sequence. In certain preferred embodiments, resulting amplicons are short to allow for rapid cycling and generation of copies. The size of the amplicon can vary as needed, for example, to provide the ability to discriminate target nucleic acids from non-target nucleic acids. For example, amplicons can be less than about 1,000 nucleotides in length. In some embodiments, the amplicons are from 100 to 500 nucleotides in length (e.g., 100 to 200, 150 to 250, 300 to 400, 350 to 450, or 400 to 500 nucleotides in length). In other embodiments, the amplicons are greater than about 1,000 nucleotides in length, e.g., about 1,000, about 2,000, about 3,000, about 4,000, about 5,000, about 6,000, about 7,000, about 8,000, about 9,000, about 10,000, or more nucleotides in length. In some embodiments, more than one (e.g., 1, 2, 3, 4, 5, 6, 7, 8, 9, 10, or more than 10) target nucleic acids may be amplified in one reaction. In other embodiments, a single target nucleic acid may be amplified in one reaction. In some embodiments, the invention provides amplification-based nucleic acid detection assays conducted in complex samples containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, CSF, BAL, SF, or sputum (e.g., purulent sputum or bloody sputum). In several embodiments, the method provides methods for amplifying target nucleic acids in a biological sample that includes cells, cell debris, and/or nucleic acids (e.g., DNA or RNA (e.g., mRNA)) derived from both a host mammalian subject and from a microbial organism, particularly a microbial pathogen. The resulting amplified target nucleic acids, or portions or fragments thereof, can be sequenced according to any of the sequencing approaches known in the art and/or described herein.

Sample preparation typically involves removing or providing resistance for common PCR inhibitors found in complex samples containing cells and/or cell debris. Common inhibitors are listed in Table A (see also Wilson, Appl. Environ. Microbiol., 63:3741 (1997)). The “facilitators” in Table A indicate methodologies or compositions that may be used to reduce or overcome inhibition. Any of the facilitators may be used in the methods described herein. Inhibitors typically act by either prevention of cell lysis, degradation or sequestering a target nucleic acid, and/or inhibition of a polymerase activity. The most commonly employed polymerase, Taq, is typically inhibited by the presence of 0.1% blood in a reaction. Mutant Taq polymerases have been engineered that are resistant to common inhibitors (e.g., hemoglobin and/or humic acid) found in blood (see, e.g., Kermekchiev et al., Nucl. Acid. Res., 37(5): e40, (2009)). Manufacturer recommendations indicate these mutations enable direct amplification from up to 20% blood.

TABLE A
PCR inhibitors and facilitators for overcoming inhibition

| Sample or | | | |
|---|---|---|---|
| Specimen Type | Target | Inhibitor | Facilitator |
| feces | Escherichia coli | >10³ bacterial cells | ion-exchange column |
| CSF | Treponema | Cell debris causing nonspecific | nested primers |
| | pallidum | amplification | |
| whole blood | mammalian | >4 μl of blood/100-ml reaction | 1-2% blood per reaction |
| | tissue | mix | |
| | | (hemoglobin) | |
| feces | Rotavirus | unknown dilution | cellulose fiber |
| clinical | Cytomegalovirus | unidentified components | glass bead extraction |
| specimens | | | |
| human blood | human genes | DNA binding proteins | thermophilic protease from |
| and tissue | | | Thermus strain rt44A |
| mammalian | Mammalian | thermal cycler variations | formamide |
| tissue | tissue genetics | | |
| mammalian | Mammalian | thermal cycler variations | DMSO, glycerol, PEG, |
| tissue | tissue genetics | | organic solvents |
| clinical | Treponema | unknown factors | Various substrate-specific |
| specimens | pallidum | | physicochemical methods |
| forensic semen | Sperm | Genotyping errors; | |
| samples | | selective/total PCR inhibition by | |
| | | vaginal microorganisms | |
| feces | Salmonella | various body fluids | immunomagnetic |
| | enterica | | separation |
| feces | Various enteric | unknown | size exclusion |
| | viruses | | chromatography, |
| | | | physicochemical extraction |
| clinical | Herpes simplex | endogenous inhibitors, random | repurification, coamplified |
| specimens | virus | effects | positive control |
| feces | Escherichia coli | nonspecific inhibitors, urea, | additional primers and |
| | | hemoglobin, heparin, phenol, | reaction cyclers, booster |
| | | SDS | PCR |
| tissue culture | Cytomegalovirus | glove powder | |
| | HIV | | |
| suspensions, | Mycobacterium | mercury-based fixatives, | reduced fixation times, |
| skin biopsies | leprae | neutral buffered formaline | ethanol fixation |
| clinical | Mycobacterium | unknown inhibitors in pus, | physicochemical extraction |
| specimens | tuberculosis | tissue biopsies, sputum, pleural | |
| | | fluid | |
| mammalian | mammalian | unknown contaminant of | additional DNA |
| tissue | tissue genetics | reverse transcriptase | |
| formalin-fixed | Hepatitis C virus | ribonucleotide vanadyl | phenol/chloroform |
| paraffin tissue | | complexes | extraction |
| nasopharyngeal | Bordetella | unknown inhibitors | phenol/chloroform |
| aspirates and | pertussis | | extraction |
| swabs | | | |
| human | HIV type 1 | detergents | mineral oil |
| mononuclear | | | |
| blood cells | | | |
| bloodstain | human | unidentified heme compound, | BSA |
| | mitochondrial | hemin | |
| | DNA | | |
| blood | various | heparin | alternative polymerases |
| | | | and buffers, chelex, |
| | | | spermine, [Mg2+], glycerol, |
| | | | BSA, heparinase |
| sputum | Mycoplasma | N-acetyl-L-cysteine, | |
| | pneumoniae | dithiothreitol, mucolytic agents | |
| human tissue | HLA-DRB1 | pollen, glove powder,impure | |
| | genotyping | DNA, heparin, hemoglobin | |
| clinical | Mycobacterium | unknown | competitive internal control |
| specimens | tuberculosis | | |
| dental plaque | many | unknown | diatomaceous earth, |
| | | | guanidium isothiocyante, |
| | | | ethanol, acetone |
| ancient | Cytochrome b | unknown | ammonium acetate, |
| mammalian | gene | | ethidium bromide |
| tissues | | | |

Polymerase chain reaction (PCR) amplification of DNA or cDNA is a tried and trusted methodology; however, as discussed above, polymerases are inhibited by agents contained in complex biological samples containing cells, cell debris, and/or nucleic acids (e.g., DNA or RNA)), including but not limited to commonly used anticoagulants and hemoglobin. Recently, mutant Taq polymerases have been engineered to harbor resistance to common inhibitors found in blood and soil. Currently available polymerases, e.g., HemoKlenTaq® (New England BioLabs, Inc., Ipswich, Mass.) as well as OmniTaq® and OmniKlenTaq® (DNA Polymerase Technology, Inc., St. Louis, Mo.) are mutant (e.g., N-terminal truncation and/or point mutations) Taq polymerase that render them capable of amplifying DNA in the presence of up to 10%, 20% or 25% whole blood, depending on the product and reaction conditions (See, e.g., Kermekchiev et al. Nucl. Acids Res. 31:6139 (2003); and Kermekchiev et al., Nucl. Acid. Res., 37:e40 (2009); and see U.S. Pat. No. 7,462,475). Additionally, PHUSION® Blood Direct PCR Kits (Finnzymes Oy, Espoo, Finland), include a unique fusion DNA polymerase enzyme engineered to incorporate a double-stranded DNA binding domain, which allows amplification under conditions which are typically inhibitory to conventional polymerases such as Taq or Pfu, and allow for amplification of DNA in the presence of up to about 40% whole blood under certain reaction conditions. See Wang et al., Nucl. Acids Res. 32:1197 (2004); and see U.S. Pat. Nos. 5,352,778 and 5,500,363. Furthermore, Kapa Blood PCR Mixes (Kapa Biosystems, Woburn, Mass.), provide a genetically engineered DNA polymerase enzyme which allows for direct amplification of whole blood at up to about 20% of the reaction volume under certain reaction conditions. Despite these breakthroughs, direct optical detection of generated amplicons is typically not possible with existing methods since fluorescence, absorbance, and other light based methods yield signals that are quenched by the presence of blood. See Kermekchiev et al., Nucl. Acid. Res., 37:e40 (2009).

Table B shows a list of mutant thermostable DNA polymerases that are compatible with many types of interfering substances and that may be used in the methods of the invention for amplification of target nucleic acids in biological samples containing cells and/or cell debris. In certain embodiments, the invention features the use of enzymes compatible with whole blood, e.g., mutant thermostable DNA polymerases including but not limited to NEB HemoKlenTaq™, DNAP OmniKlenTaq™, Kapa Biosystems whole blood enzyme, Thermo-Fisher Finnzymes PHUSION® enzyme, or any of the mutant thermostable DNA polymerases shown in Table B.

TABLE B
Exemplary mutant thermostable DNA polymerases

| Polymerase | Reference |
|---|---|
| Klentaq ®1 | Barnes, Proc Natl Acad Sci USA. 91 (6): 2216-2220, 1994. |
| Klentaq ® LA | ″ |
| Cesium Klentaq ® AC | Kermekchiev et al., Nuc. Acids Res. 31(21): 6139-6147, 2003. |
| Cesium Klentaq ® AC LA | ″ |
| Cesium Klentaq ® C | ″ |
| Cesium Klentaq ® C LA | ″ |
| Omni Klentaq ® | Kermekchiev et al. Nuc. Acids Res. 37(5):e40, 2009. |
| Omni Klentaq ® 2 | ″ |
| Omni Klentaq ® LA | ″ |
| Omni Taq | ″ |
| Omni Taq LA | ″ |
| Omni Taq 2 | ″ |
| Omni Taq 3 | ″ |
| Hemo KlenTaq ® | ″ |
| KAPA Blood DNA | KAPA Biosystems |
| Polymerase | |
| KAPA3G Plant DNA | KAPA Biosystems |
| Polymerase | |
| KAPA2G Robust DNA | KAPA Biosystems |
| Polymerase | |
| MyTaq ™ Blood-PCR Kit | Bioline |
| Phusion ® Blood DNA Kit | Thermo Scientific |
| with Hot Start Phusion II | Manage et al., Microfluid. Nanofluid. 10, 697-702, 2011. |

As described above, a variety of impurities and components of whole blood can be inhibitory to the polymerase and primer annealing. These inhibitors can sometimes lead to generation of false positives and low sensitivities. To reduce the generation of false positives and low sensitivities when amplifying and detecting nucleic acids in complex samples, it is desirable to utilize a thermal stable polymerase not inhibited by whole blood samples, for example as described above, and include one or more internal PCR assay controls (see Rosenstraus et al. J. Clin Microbiol. 36:191 (1998) and Hoofar et al., J. Clin. Microbiol. 42:1863 (2004)).

For example, the assay can include an internal control nucleic acid that contains primer binding regions identical to those of the target sequence to assure that clinical specimens are successfully amplified and detected. In some embodiments, the target nucleic acid and internal control can be selected such that each has a unique probe binding region that differentiates the internal control from the target nucleic acid. The internal control is, optionally, employed in combination with a processing positive control, a processing negative control, and a reagent control for the safe and accurate determination and identification of an infecting organism in, e.g., a whole blood clinical sample. The internal control can be an inhibition control that is designed to co-amplify with the nucleic acid target being detected. Failure of the internal inhibition control to be amplified is evidence of a reagent failure or process error. Universal primers can be designed such that the target sequence and the internal control sequence are amplified in the same reaction tube. Thus, using this format, if the target DNA is amplified but the internal control is not it is then assumed that the target DNA is present in a proportionally greater amount than the internal control and the positive result is valid as the internal control amplification is unnecessary. If, on the other hand, neither the internal control nor the target is amplified it is then assumed that inhibition of the PCR reaction has occurred and the test for that particular sample is not valid.

The assays of the invention can include one or more positive processing controls in which one or more target nucleic acids is included in the assay (e.g., each included with one or more cartridges) at 3× to 5× the limit of detection. If detected by T2MR, the measured T₂ for each of the positive processing controls must be above the pre-determined threshold indicating the presence of the target nucleic acid. The positive processing controls can detect all reagent failures in each step of the process (e.g., lysis, PCR, and T2MR detection), and can be used for quality control of the system. The assays of the invention can include one or more negative processing controls consisting of a solution free of target nucleic acid (e.g., buffer alone). If detected by T2MR, the T₂ measurements for the negative processing control should be below the threshold indicating a negative result while the T₂ measured for the internal control is above the decision threshold indicating an internal control positive result. The purpose of the negative control is to detect carry-over contamination and/or reagent contamination. The assays of the invention can include one or more reagent controls. The reagent control will detect reagent failures in the PCR stage of the reaction (i.e. incomplete transfer of master mix to the PCR tubes). The reagent controls can also detect gross failures in reagent transfer prior to T₂ detection.

The methods of the invention can also include use of a total process control (TPC), for example, an engineered cell (e.g., an engineered bacterium or fungus (e.g., yeast)) comprising a control target nucleic acid that has a known and defined sequence. The TPC may be added to the sample (e.g., environmental or biological sample) as a control to monitor steps including cell lysis, amplification, and sequencing.

In some embodiments, complex samples, which may be a liquid sample (including, for example, a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum) can be directly amplified using about 5%, about 10%, about 20%, about 25%, about 30%, about 25%, about 40%, about 45%, about 50%, about 55%, about 60%, about 65%, about 70%, or more complex liquid sample in amplification reactions, and that the resulting amplicons can be directly detected from amplification reaction using, for example, sequencing (e.g., massively parallel, long-read, and/or Sanger sequencing) and/or magnetic resonance (MR) relaxation measurements (e.g., T2MR) upon the addition of conjugated magnetic particles bound to oligonucleotides complementary to the target nucleic acid sequence. Alternatively, the magnetic particles can be added to the sample prior to amplification. Thus, provided are methods for the use of nucleic acid amplification in a complex dirty sample, sequencing and/or hybridization of the resulting amplicon to paramagnetic particles, which may be followed by direct detection of hybridized magnetic particle conjugate and target amplicons using magnetic particle based detection systems. In some embodiments, the detection is by sequencing only. In other embodiments, direct detection of hybridized magnetic particle conjugates and amplicons is via MR relaxation measurements (e.g., T₂, T₁, T₁/T₂ hybrid, T₂*, and the like). Further provided are methods which are kinetic, in order to quantify the original nucleic acid copy number within the sample (e.g., sampling and nucleic acid detection at pre-defined cycle numbers, comparison of endogenous internal control nucleic acid, use of exogenous spiked homologous competitive control nucleic acid). In some embodiments, the resulting amplicons are detected using a non-MR-based approach, for example, optical, fluorescent, mass, density, chromatographic, and/or electrochemical measurement.

While the exemplary methods described hereinafter relate to amplification using PCR, numerous other methods are known in the art for amplification of nucleic acids (e.g., isothermal methods, rolling circle methods, and the like). Those skilled in the art will understand that these other methods may be used either in place of, or together with, PCR methods. See, e.g., Saiki, “Amplification of Genomic DNA” in PCR Protocols, Innis et al., Eds., Academic Press, San Diego, Calif., pp 13-20 (1990); Wharam et al., Nucleic Acids Res. 29:E54 (2001); Hafner et al., Biotechniques, 30:852 (2001). Further amplification methods suitable for use with the present methods include, for example, reverse transcription PCR (RT-PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, ramification amplification (RAM), transcription based amplification system (TAS), transcription mediated amplification (TMA), the isothermal and chimeric primer-initiated amplification of nucleic acid (ICAN) method, and the smart amplification system (SMAP) method. These methods, as well as others are well known in the art and can be adapted for use in conjunction with provided methods of detection of amplified nucleic acid.

The PCR method is a technique for making many copies of a specific template DNA sequence. The PCR process is disclosed, for example, in U.S. Pat. Nos. 4,683,195; 4,683,202; and 4,965,188. One set of primers complementary to a template DNA are designed, and a region flanked by the primers is amplified by DNA polymerase in a reaction including multiple amplification cycles. Each amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan et al., Journal of Clinical Microbiology 33:556 (1995). Various modified PCR methods are available and well known in the art. Various modifications such as the “RT-PCR” method, in which DNA is synthesized from RNA using a reverse transcriptase before performing PCR; and the “TaqMan® PCR” method, in which only a specific allele is amplified and detected using a fluorescently labeled TaqMan® probe, and Taq DNA polymerase, are known to those skilled in the art. RT-PCR and variations thereof have been described, for example, in U.S. Pat. Nos. 5,804,383; 5,407,800; 5,322,770; and 5,310,652, and references described therein; and TaqMan® PCR and related reagents for use in the method have been described, for example, in U.S. Pat. Nos. 5,210,015; 5,876,930; 5,538,848; 6,030,787; and 6,258,569.

In some embodiments, asymmetric PCR is performed to preferentially amplify one strand of a double-stranded DNA (dsDNA) template. Asymmetric PCR typically involves addition of an excess of the primer for the strand targeted for amplification. An exemplary asymmetric PCR condition is 300 nM of the excess primer and 75 nM of the limiting primer to favor single strand amplification. In other embodiments, 400 nM of the excess primer and 100 nM of the limiting primer may be used to favor single strand amplification. In other embodiments, symmetric PCR is performed.

In some embodiments, including embodiments that employ multiplexed PCR reactions, hot start PCR conditions may be used to reduce mis-priming, primer-dimer formation, improve yield, and/or and ensure high PCR specificity and sensitivity. A variety of approaches may be employed to achieve hot start PCR conditions, including hot start DNA polymerases (e.g., hot start DNA polymerases with aptamer-based inhibitors or with mutations that limit activity at lower temperatures) as well as hot start dNTPs (e.g., CLEANAMP™ dNTPs, TriLink Biotechnologies).

In some embodiments, a PCR reaction may include from about 20 cycles to about 55 cycles or more (e.g., about 20, 25, 30, 35, 40, 45, 50, or 55 cycles).

LCR is a method of DNA amplification similar to PCR, except that it uses four primers instead of two and uses the enzyme ligase to ligate or join two segments of DNA. Amplification can be performed in a thermal cycler (e.g., LCx of Abbott Labs, North Chicago, Ill.). LCR can be performed for example, as according to Moore et al., Journal of Clinical Microbiology 36:1028 (1998). LCR methods and variations have been described, for example, in European Patent Application Publication No. EP0320308, and U.S. Pat. No. 5,427,930.

The TAS method is a method for specifically amplifying a target RNA in which a transcript is obtained from a template RNA by a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173 (1989). The TAS method has been described, for example, in International Patent Application Publication No. WO1988/010315.

Transcription mediated amplification (TMA) is a transcription-based isothermal amplification reaction that uses RNA transcription by RNA polymerase and DNA transcription by reverse transcriptase to produce an RNA amplicon from target nucleic acid. TMA methods are advantageous in that they can produce 100 to 1000 copies of amplicon per amplification cycle, as opposed to PCR or LCR methods that produce only 2 copies per cycle. TMA has been described, for example, in U.S. Pat. No. 5,399,491.

NASBA is a transcription-based method which for specifically amplifying a target RNA from either an RNA or DNA template. NASBA is a method used for the continuous amplification of nucleic acids in a single mixture at one temperature. A transcript is obtained from a template RNA by a DNA-dependent RNA polymerase using a forward primer having a sequence identical to a target RNA and a reverse primer having a sequence complementary to the target RNA a on the 3′ side and a promoter sequence that recognizes T₇ RNA polymerase on the 5′ side. A transcript is further synthesized using the obtained transcript as template. This method can be performed as according to Heim, et al., Nucleic Acids Res., 26:2250 (1998). The NASBA method has been described in U.S. Pat. No. 5,130,238.

The SDA method is an isothermal nucleic acid amplification method in which target DNA is amplified using a DNA strand substituted with a strand synthesized by a strand substitution type DNA polymerase lacking 5′->3′ exonuclease activity by a single stranded nick generated by a restriction enzyme as a template of the next replication. A primer containing a restriction site is annealed to template, and then amplification primers are annealed to 5′ adjacent sequences (forming a nick). Amplification is initiated at a fixed temperature. Newly synthesized DNA strands are nicked by a restriction enzyme and the polymerase amplification begins again, displacing the newly synthesized strands. SDA can be performed according to Walker, et al., PNAS, 89:392 (1992). SDA methods have been described in U.S. Pat. Nos. 5,455,166 and 5,457,027.

The LAMP method is an isothermal amplification method in which a loop is always formed at the 3′ end of a synthesized DNA, primers are annealed within the loop, and specific amplification of the target DNA is performed isothermally. LAMP can be performed according to Nagamine et al., Clinical Chemistry. 47:1742 (2001). LAMP methods have been described in U.S. Pat. Nos. 6,410,278; 6,974,670; and 7,175,985.

The ICAN method is anisothermal amplification method in which specific amplification of a target DNA is performed isothermally by a strand substitution reaction, a template exchange reaction, and a nick introduction reaction, using a chimeric primer including RNA-DNA and DNA polymerase having a strand substitution activity and RNase H. ICAN can be performed according to Mukai et al., J. Biochem. 142: 273(2007). The ICAN method has been described in U.S. Pat. No. 6,951,722.

The SMAP (MITANI) method is a method in which a target nucleic acid is continuously synthesized under isothermal conditions using a primer set including two kinds of primers and DNA or RNA as a template. The first primer included in the primer set includes, in the 3′ end region thereof, a sequence (Ac′) hybridizable with a sequence (A) in the 3′ end region of a target nucleic acid sequence as well as, on the 5′ side of the above-mentioned sequence (Ac′), a sequence (B′) hybridizable with a sequence (Bc) complementary to a sequence (B) existing on the 5′ side of the above-mentioned sequence (A) in the above-mentioned target nucleic acid sequence. The second primer includes, in the 3′ end region thereof, a sequence (Cc′) hybridizable with a sequence (C) in the 3′ end region of a sequence complementary to the above-mentioned target nucleic acid sequence as well as a loopback sequence (D-Dc′) including two nucleic acid sequences hybridizable with each other on an identical strand on the 5′ side of the above-mentioned sequence (Cc′). SMAP can be performed according to Mitani et al., Nat. Methods, 4(3): 257 (2007). SMAP methods have been described in U.S. Patent Application Publication Nos. 2006/0160084, 2007/0190531 and 2009/0042197.

The amplification reaction can be designed to produce a specific type of amplified product, such as nucleic acids that are double stranded; single stranded; double stranded with 3′ or 5′ overhangs; or double stranded with chemical ligands on the 5′ and 3′ ends. The amplified PCR product can be detected by: (i) sequencing; (ii) hybridization of the amplified product to magnetic particle bound complementary oligonucleotides, where two different oligonucleotides are used that hybridize to the amplified product such that the nucleic acid serves as an interparticle tether promoting particle agglomeration; (iii) hybridization mediated detection where the DNA of the amplified product must first be denatured; (iv) hybridization mediated detection where the particles hybridize to 5′ and 3′ overhangs of the amplified product; and/or (v) binding of the particles to the chemical or biochemical ligands on the termini of the amplified product, such as streptavidin functionalized particles binding to biotin functionalized amplified product.

### Analytes

Embodiments of the invention include methods, panels, and systems for amplifying, detecting, sequencing, and/or measuring the concentration of one or more analytes (e.g., one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids), and/or (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids)) in a complex biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., a tissue biopsy (e.g., a skin biopsy, muscle biopsy, or lymph node biopsy), including homogenized tissue samples), urine, cerebrospinal fluid (CSF), synovial fluid (SF), or sputum. In several embodiments, the analyte may be a nucleic acid derived from an organism. In some embodiments, the nucleic acid is a target nucleic acid derived from the organism that has been amplified to form an amplicon. In some embodiments, the organism is a plant, a mammal, or a microbial species. The nucleic acid can be detected by sequencing. In some embodiments, the nucleic acid may further be detected by other approaches, including T2MR.

In some embodiments, the analyte may be derived from a microbial pathogen. In such embodiments, the biological sample may include cells and/or cell debris from the host mammalian subject as well as one or more microbial pathogen cells. For example, in some embodiments, the analyte is derived from a Gram-negative bacterium, a Gram-positive bacterium, a fungal pathogen (e.g., a yeast (e.g., Candida spp.) or Aspergil/us spp.), a protozoan pathogen, or a viral pathogen.

In some embodiments, the analyte is derived from a bacterial pathogen, including Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakit), Enterobacteriaceae spp., Enterococcus spp. (e.g., Enterococcus faecium (including E. faecium with resistance marker vanA/B) and Enterococcus faecalis), Fusarium spp. (e.g. Fusarium solani), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae).

In some embodiments, the analyte is derived from a fungal pathogen, for example, Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis) and Aspergillus spp. (e.g., Aspergillus fumigatus). In some embodiments, the analyte is derived from a protozoan pathogen such as a Babesia spp. (e.g., Babesia microti and Babesia divergens). In some embodiments, the analyte is derived from a viral pathogen (e.g., a retrovirus (e.g., HIV), an adeno-associated virus (AAV), an adenovirus, Ebolavirus, hepatitis (e.g., hepatitis A, B, C, or E), herpesvirus, human papillomavirus (HPV), rhinovirus, influenza, parainfluenza, measles, rotavirus, West Nile virus, zika virus, and the like). In some embodiments, the analyte is derived from a biothreat species e.g., Bacillus anthracis, Francisella tularensis, Burkholderia spp. (e.g., B. mallei or B. pseudomallet), Yersinia pestis, or Rickettsia prowazekii. In some embodiments, the analyte is a toxin gene, e.g., Bacillus anthracis toxin genes protective antigen (pagA), edema factor (cya), or lethal factor (lef); enteropathogenic E. coli translocated intimin receptor (Tir); Clostridium difficile toxins TcdA and TcdB; or Clostridium botulinum toxins BoNT/A, BoNT/B, BoNT/C, BoNT/D, BoNT/E, BoNT/F, or BoNT/G.

In some embodiments, the analyte is an antimicrobial resistance marker. Exemplary non-limiting antimicrobial resistance markers include, e.g., bla_{KPC}, blaZ, bla_{NDM}, bla_{IMP}, bla_{VIM}, bla_{OXA}, bla_{CMY}, bla_{DHA}, bla_{TEM}, bla_{SHV}, blaC_{TX-M}, bla_{SME}, bla_{FOX}, bla_{MIR}, femA, femB, mecA, mecC, MCR, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1. In particular embodiments, the analyte includes one or more antibiotic resistance genes selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, and/or CTX-M 15), mefA, mefE, ermA, ermB, SHV, TEM, FKS, PDR1, and ERG11. In particular embodiments, the analyte includes CTX-M. In other particular embodiments, the analyte includes mcr-1.

In some embodiments, a pathogen-associated analyte may be a nucleic acid derived from any of the organisms described above, for example, DNA or RNA (e.g., mRNA). In some embodiments, the nucleic acid is a target nucleic acid derived from the organism that has been amplified to form an amplicon. In some embodiments, the target nucleic acid may be a multi-copy locus. Use of a target nucleic acid derived from a multi-copy locus, in particular in methods involving amplification, may lead to an increase in sensitivity in the assay. Exemplary multi-copy loci may include, for example, ribosomal DNA (rDNA) operons and multi-copy plasmids. In other embodiments, the target nucleic acid may be a single-copy locus. In particular embodiments, the target nucleic acid may be derived from an essential locus, for example, an essential house-keeping gene. In particular embodiments, the target nucleic acid may be derived from a locus that is involved in virulence (e.g., a virulence gene). In any of the above embodiments, a locus may include a gene and/or an intragenic region, for example, an internally transcribed sequence (ITS) between rRNA genes (e.g., ITS1, between the 16S and 23S rRNA genes, or ITS2, between the 5S and 23S rRNA genes). In some embodiments, the target nucleic acid is a 16S rRNA target nucleic acid.

In some embodiments, a target nucleic acid may be (a) species-specific, (b) species-inclusive (in other words, present in all strains or subspecies of a given species), (c) compatible with an amplification/detection protocol, and/or (d) present in multiple copies. In some embodiments, a target nucleic acid may be group-specific or group-inclusive, e.g., genus-specific or genus inclusive. In particular embodiments, a target nucleic acid is chromosomally-encoded.

### Panels

The methods and compositions (e.g., systems, devices, kits, or cartridges) described herein can be configured to detect and/or sequence target nucleic acids from a predetermined panel of targets. Any of the pathogens described herein can be detected and/or sequenced using the panels described herein.

For example, provided herein is a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids). In some embodiments, the panel includes at least 28 pathogen target nucleic acids.

In any of the panels described herein, the panel may further include (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids).

In any of the panels described herein, the panel may further include (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids).

Any of the panels described herein may have a percent coverage of greater than or equal to 90% (e.g., 90%, 91%, 92%, 93%, 94%, 95%, 96%, 97%, 98%, 99%, or 100%) of pathogen species associated with infections of the sample. For example, in some embodiments, the panels has a percent coverage of greater than or equal to 95%, 96%, 97%, 98%, or 99% of pathogen species associated with infections of the sample. In some embodiments, the panels has a percent coverage of greater than or equal to 99% of pathogen species associated with infections of the sample.

The panels described herein may be split across one or more subpanels. Any suitable number of subpanels may be used in any of the methods described herein. For example, in some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty subpanels. In some embodiments, the panel includes at least two subpanels. In some embodiments, the panel includes at least four subpanels. In some embodiments, the panel includes five subpanels.

Each subpanel may include any suitable number of pathogen target nucleic acids. For example, in some embodiments, the subpanel may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more pathogen target nucleic acids. For example, in some embodiments, the subpanel includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, or more pathogen target nucleic acids. In some embodiments, each subpanel includes at least 6 pathogen target nucleic acids. In some embodiments, each subpanel includes 9 pathogen target nucleic acids. In some embodiments, each subpanel includes 14 pathogen target nucleic acids. In some embodiments, each subpanel includes an internal control channel.

The panels described herein may include any suitable number of pathogen target nucleic acids. For example, in some embodiments, the panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, 100, or more target nucleic acids. In some embodiments, the panel includes at least 28 pathogen target nucleic acids. In some embodiments, the panel includes at least 30 pathogen target nucleic acids. In some embodiments, the panel includes at least 32 pathogen target nucleic acids. In some embodiments, the panel includes at least 36 pathogen target nucleic acids. In some embodiments, the panel includes at least 36 pathogen target nucleic acids. In some embodiments, the panel includes at least 38 pathogen target nucleic acids. In some embodiments, the panel includes at least 40 pathogen target nucleic acids. In some embodiments, the panel includes at least 42 pathogen target nucleic acids. In some embodiments, the panel includes at least 44 pathogen target nucleic acids. In some embodiments, the panel includes 45 pathogen target nucleic acids. In some embodiments, the 45 pathogen target nucleic acids are split between five subpanels.

For example, in some embodiments, the panel includes between 15 and 80, between 15 and 75, between 15 and 70, between 15 and 65, between 15 and 60, between 15 and 55, between 15 and 50, between 15 and 45, between 15 and 40, between 15 and 35, between 15 and 30, between 15 and 25, between 15 and 20, between 20 and 80, between 20 and 75, between 20 and 70, between 20 and 65, between 20 and 60, between 20 and 55, between 20 and 50, between 20 and 45, between 20 and 40, between 20 and 35, between 20 and 30, between 20 and 25, between 25 and 80, between 25 and 75, between 25 and 70, between 25 and 65, between 25 and 60, between 25 and 55, between 25 and 50, between 25 and 45, between 25 and 40, between 25 and 35, between 25 and 30, between 30 and 80, between 30 and 75, between 30 and 70, between 30 and 65, between 30 and 60, between 30 and 55, between 30 and 50, between 30 and 45, between 30 and 40, between 30 and 35, between 35 and 80, between 35 and 75, between 35 and 70, between 35 and 65, between 35 and 60, between 35 and 55, between 35 and 50, between 35 and 45, between 35 and 40, between 40 and 80, between 40 and 75, between 40 and 70, between 40 and 65, between 40 and 60, between 40 and 55, between 40 and 50, between 40 and 45, between 45 and 80, between 45 and 75, between 45 and 70, between 45 and 65, between 45 and 60, between 45 and 55, between 45 and 50, between 50 and 80, between 50 and 75, between 50 and 70, between 50 and 65, between 50 and 60, between 50 and 55, between 55 and 80, between 55 and 75, between 55 and 70, between 55 and 65, between 55 and 60, between 60 and 80, between 60 and 75, between 60 and 70, between 60 and 65, between 65 and 80, between 65 and 75, between 65 and 70, between 65 and 65, between 70 and 80, between 70 and 75, or between 75 and 80 pathogen target nucleic acids.

Any suitable genus-level target nucleic acids may be included in any of the panels described herein. For example, in some embodiments, the one or more genus-level target nucleic acids are characteristic of a genus selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp. In some embodiments, the one or more genus-level target nucleic acids are characteristic of a genus selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments, the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, or all twenty-two genus-level target nucleic acids selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty genus-level target nucleic acids selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments, the genus-level target nucleic acid characteristic of Enterobacteriaceae is characteristic of Klebsiella spp., Enterobacter spp., Citrobacter spp., Serratia spp., Proteus spp., and/or Morganella spp. In some embodiments, the genus-level target nucleic acid characteristic of coagulase negative Staphylococcus spp. is characteristic of S. epidermidis, S. haemolyticus, S. lugdunensis, and/or S. hominis. In some embodiments, the genus-level target nucleic acid characteristic of Viridans group Streptococcus is characteristic of S. anginosus, S. mitis, and/or S. oralis. In some embodiments, the genus-level target nucleic acid characteristic of anaerobes is characteristic of Clostridium spp. and/or Bacteroides spp.

In some embodiments of any of the panels described herein, the genus-level target nucleic acid may be characteristic of Staphylococcus spp., and the Staphylococcus spp. target nucleic acid is amplified in the presence of a forward primer including the nucleotide sequence of CACATTCTTTTATCACGTAACGTTGGTGT (SEQ ID NO: 179) and a reverse primer including the nucleotide sequence of CCAGGCATTACCATTTCAGTACCTTCTGGTAA (SEQ ID NO: 180) to produce a Staphylococcus spp. amplicon. In some embodiments, a probe pair including a 5′ probe including the nucleotide sequence of CCAGTTACGTCAGTAGTACGGAA (SEQ ID NO: 181) and a 3′ probe including the nucleotide sequence of TTTGATTTGACCACGTTCAACAC (SEQ ID NO: 182) is used for detection of the Staphylococcus spp. amplicon.

In some embodiments of any of the panels described herein, the genus-level target nucleic acid may be characteristic of Candida spp., and the Candida spp. target nucleic acid is amplified in the presence of a forward primer including a nucleotide sequence selected from the group consisting of GGCATGCCTGTTTGAGCGTC (SEQ ID NO: 93), GGCATGCCTGTTTGAGCGT (SEQ ID NO: 157), and GGGCATGCCTGTTTGAGCGT (SEQ ID NO: 159), and a reverse primer including the nucleotide sequence of GCTTATTGATATGCTTAAGTTCAGCGGGT (SEQ ID NO: 94) to produce a Candida spp. amplicon.

Any suitable Gram positive bacterial target nucleic acid may be included in any of the panels described herein. In some embodiments, the one or more Gram positive bacterial target nucleic acids are selected from the group consisting of E. faecium, E. faecalis, S. aureus, S. pneumoniae, S. pyogenes, and S. agalactiae. In some embodiments, the panel includes at least two, at least three, at least four, at least five, or all six Gram positive bacterial target nucleic acids selected from the group consisting of E. faecium, E. faecalis, S. aureus, S. pneumoniae, S. pyogenes, and S. agalactiae. In some embodiments, the one or more Gram positive bacterial target nucleic acids is amplified in the presence of a forward primer and a reverse primer set forth in Table 3. In some embodiments, the one or more Gram positive bacterial target nucleic acid amplicons is detected using a 5′ capture probe and a 3′ capture probe set forth in Table 4.

Any suitable Gram negative bacterial target nucleic acid may be included in any of the panels described herein. In some embodiments, the one or more Gram negative bacterial target nucleic acids are selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the panel includes at least two, at least three, at least four, at least five, at least six, at least seven, at least eight, at least nine, or all ten Gram negative bacterial target nucleic acids selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the one or more Gram negative bacterial target nucleic acids is amplified in the presence of a forward primer and a reverse primer set forth in Table 3. In some embodiments, the one or more Gram negative bacterial target nucleic acid amplicons is detected using a 5′ capture probe and a 3′ capture probe set forth in Table 4.

Any suitable resistance gene target nucleic acid may be included in any of the panels described herein. In some embodiments, the one or more resistance gene target nucleic acids are selected from the group consisting of mecA, mecC, mefA, mefE, MCR (e.g., mcr-1), vanA, vanB, ermA, ermB, KPC, NDM, VIM, IMP, OXA-23-like, OXA-48-like, SHV, CMY, DHA, CTX-M (e.g., CTX-M-2, CTX-M-8, CTX-M 14, and/or CTX-M 15), TEM, FKS, PDR1, and ERG11. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or all twenty-four resistance gene target nucleic acids selected from the group consisting of mecA, mecC, mefA, mefE, MCR, vanA, vanB, ermA, ermB, KPC, NDM, VIM, IMP, OXA-23-like, OXA-48-like, SHV, CMY, DHA, CTX-M 14, CTX-M 15, TEM, FKS, PDR1, and ERG11. In some embodiments, the resistance gene target nucleic acid is characteristic of mecA and mecC; mefA and mefE; vanA and vanB; ermA and ermB; NDM, VIM, and IMP; CMY and DHA; or CTX-M 14 and CTX M 15. In some embodiments, the one or more resistance gene target nucleic acids is amplified in the presence of a forward primer and a reverse set forth in Table 10, Table 12, Table 14, Table 16, or Table 26. In some embodiments, the one or more resistance gene target nucleic acids is amplified in the presence of a forward primer and a reverse set forth in Table 26. In some embodiments, the one or more resistance gene target nucleic acids is amplified in the presence of a forward primer and a reverse set forth in Table 10, Table 12, Table 14, or Table 16. In some embodiments, the one or more resistance gene target nucleic acid amplicons is detected using a 5′ capture probe and a 3′ capture probe set forth in Table 11, Table 13, Table 15, Table 17, or Table 26. In some embodiments, the one or more resistance gene target nucleic acid amplicons is detected using a 5′ capture probe and a 3′ capture probe set forth in Table 26. In some embodiments, the one or more resistance gene target nucleic acid amplicons is detected using a 5′ capture probe and a 3′ capture probe set forth in Table 11, Table 13, Table 15, or Table 17.

In some embodiments, the resistance target nucleic acid characteristic of CTX-M is a universal CTX-M target nucleic acid. In some embodiments, the universal CTX-M target nucleic acid is characteristic of CTX-M-2, CTX-M-8, CTX-M-14, and CTX-M-15. In some embodiments, the universal CTX-M target nucleic acid is amplified in the presence of (i) a first degenerate forward primer comprising the nucleotide sequence of CGTTTTCCIATGTGCAGTACCAGTAAGGTTATGGC (SEQ ID NO: 285) and a second degenerate forward primer comprising the nucleotide sequence of CGTTTTGCIATGTGCAGTACCAGTAAGGTGATGGC (SEQ ID NO: 286) and (ii) a first degenerate reverse primer comprising the nucleotide sequence of GGTGAGGTGGTGTCGCGCGGGTCGCCIGGGAT (SEQ ID NO: 287) and a second degenerate reverse primer comprising the nucleotide sequence of GGTGAGGTGGTGTCTCTCGGGTCGCCIGGGAT (SEQ ID NO: 288). In some embodiments, a plurality of probes comprising (i) a plurality of 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a plurality of 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291) are used for detection of the universal CTX-M amplicon. In some embodiments, a first population of 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a second population of 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291), are used for detection of the universal CTX-M amplicon.

In some embodiments of any of the panels described herein, the resistance gene target nucleic acid may be OXA-23-like, and the OXA-23-like target nucleic acid is amplified in the presence of a forward primer including the nucleotide sequence of AGATTGTTCAAGGACATAATCAGGTGA (SEQ ID NO: 183) and a reverse primer including the nucleotide sequence of GGTAAATGACCTTTTCTCGCCCTTC (SEQ ID NO: 184) to produce a OXA-23-like amplicon. In some embodiments, a probe pair including a 5′ probe including the nucleotide sequence of CTCAGGTGTGCTGGTTATTCA (SEQ ID NO: 185) and a 3′ probe including the nucleotide sequence of GCCCTGATCGGATTGGAGAA (SEQ ID NO: 186) is used for detection of the OXA-23-like amplicon.

Any suitable pan-level target nucleic acid may be included in any of the panels described herein. In some embodiments, the one or more pan-level target nucleic acids are selected from the group consisting of Pan-Bacterial, Pan-Gram positive, Pan-Gram negative, and Pan-Fungal. In some embodiments, the panel includes at least two, at least three, or all four pan-level target nucleic acids selected from the group consisting of Pan-Bacterial, Pan-Gram positive, Pan-Gram negative, and Pan-Fungal.

In any of the methods described herein, on some embodiments, the Pan-Bacterial target nucleic acid may be amplified in the presence of a forward primer including the nucleotide sequence of CTCCTACGGGAGGCAGCAGT (SEQ ID NO: 173) and a reverse primer including the nucleotide sequence of GTATTACCGCGGCTGCTGGCA (SEQ ID NO: 174) to produce a Pan-Bacteria amplicon. In some embodiments, a probe pair including a 5′ probe including the nucleotide sequence of CTGACGGAGCAACGCCGCGTGAGTGA (SEQ ID NO: 175) and a 3′ probe including the nucleotide sequence of CTAACCAGAAAGCCACGGCTAACTACG (SEQ ID NO: 176) is used to detect the presence of Gram positive bacteria. In some embodiments, a probe pair including a 5′ probe including the nucleotide sequence of CTGATCCAGCCATGCCGCGTGTATGA (SEQ ID NO: 177) and a 3′ probe including the nucleotide sequence of CCGCAGAAGAAGCACCGGCTAACTCCG (SEQ ID NO: 178) is used to detect the presence of Gram negative bacteria.

Any suitable fungal target nucleic acid may be included in any of the panels described herein. In some embodiments, the one or more fungal target nucleic acids are selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the panel includes at least two, at least three, at least four, at least five, at least six, at least seven, at least eight, at least nine, at least ten, or all eleven fungal target nucleic acids selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii. In some embodiments, the one or more fungal target nucleic acids is amplified in the presence of a forward primer and a reverse primer set forth in Table 7. In some embodiments, the one or more fungal target nucleic acid amplicons is detected using a 5′ capture probe and a 3′ capture probe set forth in Table 8 or Table 9.

In some embodiments, the panel is a panel shown in any one of Tables 20-24 or in Table 27. In some embodiments, the panel is shown in Table 27.

One preferred panel includes: (i) a first subpanel including the following pathogen target nucleic acids: Pan Gram negative, E. coli, K. pneumoniae, Enterobacter spp., Enterobacter cloacae complex, Citrobacter spp., S. marcescens, P. mirabilis, Salmonella spp., and an internal control; (ii) a second subpanel including the following pathogen target nucleic acids: Acinetobacter spp., A. baumanii, P. aeruginosa, S. maltophilia, H. influenzae, KPC, NDM/VIM/IMP, OXA-48-like, CTX-M 14/15, and an internal control; (iii) a third subpanel including the following pathogen target nucleic acids: Pan Gram positive, Enterococcus spp., E. faecium, E. faecalis, Staphylococcus spp., S. aureus, coagulase negative Staphylococcus spp., mecA/C, vanA/B, and an internal control; (iv) a fourth subpanel including the following pathogen target nucleic acids: Streptococcus spp., S. pneumoniae, S. pyogenes, S. agalactiae, Viridans Group Streptococcus, Anaerobes, Corynebacterium spp., ermA/B, mefA/E, and an internal control; and (v) a fifth subpanel including the following pathogen target nucleic acids: Candida spp., C. albicans, C. tropicalis, C. parapsilosis, C. krusei, C. glabrata, C. auris, Aspergillus spp., Cryptococcus spp., and an internal control.

In some embodiments, the panel includes one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 38, 39, or all 40), of Pan Gram Positive, Pan Gram Negative, Staphylococcus aureus, Coagulase negative staphylococci, Enterococcus spp., Enterococcus faecium, Enterococcus faecalis, Streptococcus pneumoniae, Streptococcus agalactiae, Streptococcus pyogenes, Clostridium spp., Mycobacterium spp., Enterobacterales, Escherichia coli, Klebsiella pneumoniae, Klebsiella aerogenes, Enterobacter cloacae complex, Citrobacter spp., Serratia spp., Proteus spp., Acinetobacter baumannii, Bacteroides spp., Haemophilus influenzae, Pseudomonas aeruginosa, Stenotrophomonas maltophilia, mecA, mecC, vanA/B, mefA/E, KPC, NDM, VIM, IMP, OXA-48, OXA-23, OXA-24/40, CTX-M, AmpC, mcr-1, and strA/strB.

In some embodiments, the panel includes one or more pathogen target nucleic acids characteristic of one or more, or all, of the following: any bacterium (pan-bacterial), any Gram positive bacterium (pan-Gram positive), any Gram negative bacterium (pan-Gram negative), Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakit), Enterobacteriaceae spp., Enterococcus spp. (e.g., Enterococcus faecium (including E. faecium with resistance marker vanA/B) and Enterococcus faecalis), Fusarium spp. (e.g., Fusarium solani), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae), any fungal pathogen (pan-fungal), Candida spp. (e.g., Candida albicans, Candida guilliermondii, Candida glabrata, Candida krusei, Candida lusitaniae, Candida parapsilosis, Candida dublinensis, and Candida tropicalis), Aspergillus spp. (e.g., Aspergillus fumigatus) and/or a Cryptococcus spp. In some embodiments, the panel is further configured to detect a Candida spp. (including Candida albicans, Candida guilliermondii, Candida glabrata, Candida krusei, Candida lusitaniae, Candida parapsilosis, Candida dublinensis, and Candida tropicalis). In cases where multiple species of a genus are detected, the species may be detected using individual target nucleic acids or using target nucleic acids that are universal to all or nearly all of the species, for example, target nucleic acids amplified using universal primers.

Any of the preceding panels may be further configured to configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Escherichia coli, and Staphylococcus aureus. For example, in some embodiments, the panel may be configured to individually detect one or more (e.g., 1, 2, 3, 4, or 5) of Enterococcus faecium, Klebsiella pneumoniae, Pseudomonas aeruginosa, Escherichia coli, and Staphylococcus aureus, as in the FDA-cleared T2BACTERIA® panel (T2 Biosystems, Inc.).

Any of the preceding panels may be further configured to individually detect one or more (e.g., 1, 2, 3, 4, 5, 6, or 8) Candida spp. (e.g., Candida albicans, Candida guilliermondii, Candida glabrata, Candida krusei, Candida lusitaniae, Candida parapsilosis, Candida dublinensis, and Candida tropicalis). For example, in some embodiments, the panel may be configured to individually detect one or more (e.g., 1, 2, 3, 4, or 5) of Candida albicans, Candida tropicalis, Candida krusei, Candida glabrata, and Candida parapsilosis, as in the FDA-cleared T2CANDIDA® panel (T2 Biosystems, Inc.).

In some embodiments, the panel can be further configured to individually detect one, two, or three Borrelia burgdorferi sensu lato (Borrelia burgdorferi, Borrelia afzelii, and Borrelia garinii) species. These species may be detected using individual target nucleic acids or using target nucleic acids that are universal to all three species, for example, target nucleic acids amplified using universal primers. In some embodiments, the panel is configured to detect Borrelia burgdorferi. In some embodiments, the panel is configured to detect Borrelia afzelii. In some embodiments, the panel is configured to detect Borrelia garinii. In some embodiments, the panel is configured to detect Borrelia burgdorferi and Borrelia afzelii. In some embodiments, the panel is configured to detect Borrelia burgdorferi and Borrelia garinii. In some embodiments, the panel is configured to detect Borrelia afzelii and Borrelia garinii. In some embodiments, the panel is configured to detect Borrelia burgdorferi, Borrelia afzelii and Borrelia garinii. In some embodiments, the panel may be configured to individually detect one or more (e.g., 1, 2, 3, 4, 5, or 6) of Rickettsia rickettsii, Coxiella burnettii, Ehrlichia chaffeensis, Babesia microti, Francisella tularensis, and Anaplasma phagocytophilum.

In any of the above panels, the analyte may be a nucleic acid (e.g., an amplified target nucleic acid, as described above), or a polypeptide (e.g., a polypeptide derived from the pathogen or a pathogen-specific antibody produced by a host subject, for example, an IgM or IgG antibody). In some embodiments, multiple analytes (e.g., multiple amplicons) are used to detect a target, e.g., a pathogen or a drug resistance (e.g., antibiotic resistance) marker (e.g., an antibiotic resistance gene selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, and/or CTX-M 15), mefA, mefE, ermA, ermB, SHV, TEM, FKS, PDR1, and ERG11). In any of the above panels, the biological sample may be a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), BAL, urine, or sputum. In some embodiments, the biological sample is blood (e.g., whole blood, a crude whole blood lysate, serum, or plasma). Such panels may be used, for example, to diagnose bloodstream infections and/or to select an optimized therapy, e.g., by selecting an antimicrobial agent for which the pathogen is predicted to be sensitive rather than resistant. In some embodiments, the biological sample may be a tissue sample, for example, a homogenized tissue sample. Such panels may be used, for example, to detect infections present in tissue, e.g., tissue biopsies of skin at the site of a tick bite to identify Borrelia spp. for diagnosis of Lyme disease.

In some embodiments, the panel can be further configured to detect one or more toxin genes. For example, in some embodiments, the toxin gene panel can include one or more of Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G.

### Methods of Patient Identification, Selection, Treatment, and Medical Conditions

The methods, panels, and systems of the invention can also be used diagnose and monitor diseases and other medical conditions. In some embodiments, the methods, panels, and systems of the invention may be used diagnose and monitor diseases in a multiplexed, automated, no sample preparation system.

The methods, panels, and systems of the invention can be used to identify and monitor the pathogenesis of disease in a subject, to select therapeutic interventions, and to monitor the effectiveness of the selected treatment. For example, for a patient having or at risk of bacteremia and/or sepsis, the methods and systems of the invention can be used to identify the infectious drug-resistant pathogen, pathogen load, and to monitor white blood cell count and/or biomarkers indicative of the status of the infection. The presence or expression level of one or more pathogen target nucleic acids, including drug resistance markers (e.g., antibiotic resistance genes), can be used to select an appropriate therapy. The identity of the pathogen (e.g., at a pan-level, genus-level, and/or a species-level) can be used to select an appropriate therapy. In some embodiments, the methods may further include administering a therapeutic agent following monitoring or diagnosing an infectious disease. The therapeutic intervention (e.g., a particular drug such as an antibiotic agent) can be monitored as well to correlate the treatment regimen to the circulating concentration of antibiotic agent and pathogen load to ensure that the patient is responding to treatment.

For example, provided herein is a method for identifying a patient infected with a pathogen, the method including: (a) providing a biological sample obtained from the subject; and (b) detecting the presence of a pathogen target nucleic acid in the biological sample according to any one of the methods described herein, wherein the presence of a pathogen target nucleic in the biological sample obtained from the subject identifies the subject as one who may be infected with the pathogen.

Any of the methods may further include selecting an optimized therapy for the patient based on the presence of the pathogen and/or the presence of one or more resistance genes.

Any of the methods may further include administering the optimized therapy to the patient.

In some embodiments, the method results in administration of an optimized therapy to the patient faster than standard of care.

Any of the method may result in de-escalation of empiric therapy, e.g., within 10, 9, 8, 7, 6, 5, 4, 3, 2, or 1 hours. In some embodiments, the method results in de-escalation of empiric therapy within 3 to 5 hours.

Exemplary diseases that can be diagnosed, monitored, and/or treated by the methods and systems of the invention include diseases caused by or associated with microbial pathogens (e.g., bacterial infection, fungal infection, protozoal infection, or viral infection (e.g., COVID-19)), Lyme disease, bloodstream infection (e.g., bacteremia or fungemia (e.g., Candidemia)), pneumonia, peritonitis, osteomyeletis, meningitis, empyema, urinary tract infection, sepsis, septic shock, and septic arthritis) and diseases that may manifest with similar symptoms to diseases caused by or associated with microbial pathogens (e.g., SIRS). In any of the methods, the causative pathogen may be a drug-resistant pathogen (e.g., an antibiotic-resistant bacterium (e.g., an antibiotic-resistant Gram positive or Gram negative bacterium) or a drug-resistant fungal pathogen (e.g., drug-resistant Candida spp. such as drug-resistant C. auris).

For example, the methods and systems of the invention may be used to diagnose, monitor, and/or treat a disease caused by the following non-limiting examples of pathogens: bacterial pathogens, including Acinetobacter spp. (e.g., Acinetobacter baumannii, Acinetobacter pittii, and Acinetobacter nosocomialis), Bacillus spp., (including Bacillus anthracis, Bacillus cereus group, and Bacillus subtilis group), Cronobacter spp. (e.g., Cronobacter sakazakii), Enterobacteriaceae spp., Enterococcus spp. (e.g., Enterococcus faecium (including E. faecium with resistance marker vanA/B) and Enterococcus faecalis), Fusarium spp. (e.g., Fusarium solani), Fusobacterium spp. (e.g., Fusobacterium nucleatum and Fusobacterium necrophorum), Klebsiella spp. (e.g., Klebsiella pneumoniae (e.g., K. pneumoniae with resistance marker KPC), Klebsiella variicola, Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosus group, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Lactobacillus spp., Listeria spp. (e.g., Listeria monocytogenes), Micrococcus spp., Mycobacterium spp., Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morganit), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae); and fungal pathogens including but not limited to Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis) and Aspergillus spp. (e.g., Aspergillus fumigatus). In some embodiments, the pathogen may be a Borrelia spp., including Borrelia burgdorferi sensu lato (Borrelia burgdorferi, Borrelia afzelii, and Borrelia garinii) species, Borrelia americana, Borrelia andersonii, Borrelia bavariensis, Borrelia bissettii, Borrelia carolinensis, Borrelia californiensis, Borrelia chilensis, Borrelia genomosp. 1 and 2, Borrelia japonica, Borrelia kurtenbachii, Borrelia lusitaniae, Borrelia myomatoii, Borrelia sinica, Borrelia spielmanii, Borrelia tanukii, Borrelia turdi, Borrelia valaisiana and unclassified Borrelia spp. In other embodiments, the pathogen may be selected from the following: Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Babesia spp. (including Babesia microti and Babesia divergens), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the pathogen is a viral pathogen (e.g., a retrovirus (e.g., HIV), an adeno-associated virus (AAV), an adenovirus, Ebolavirus, hepatitis (e.g., hepatitis A, B, C, or E), herpesvirus, human papillomavirus (HPV), rhinovirus, influenza, parainfluenza, measles, rotavirus, West Nile virus, zika virus, and the like). In some embodiments, the pathogen is a biothreat species, e.g., Bacillus anthracis, Francisella tularensis, Burkholderia spp. (e.g., B. mallei or B. pseudomallet), Yersinia pestis, or Rickettsia prowazekii.

Any of the methods may include administering a therapeutic agent (e.g., an antimicrobial agent (e.g., an antibiotic agent)) or a composition thereof (e.g., a pharmaceutical composition) to a subject following a diagnosis or identification of a pathogen, e.g., a drug-resistant pathogen. For example, the identification of a particular pathogen and/or a particular drug resistance marker (e.g., an antibiotic resistance gene) in a biological sample obtained from the subject (e.g., a complex sample containing host cells and/or cell debris, e.g., blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, BAL, CSF, SF, or sputum) will guide the selection of the appropriate therapeutic agent (e.g., antimicrobial agent, e.g., an antibiotic, an anti-fungal agent, and the like). In other embodiments, provided herein is a method of treating a patient that includes administering a therapeutic agent (e.g., an antimicrobial agent (e.g., an antibiotic agent)) or a composition thereof (e.g., a pharmaceutical composition) to a subject, wherein the patient has been identified as being infected by a pathogen and/or having a drug resistance marker (e.g., an antibiotic resistance marker) according to any of the methods described herein.

For example, provided herein is a method of treating a patient infected with a pathogen, the method including administering an optimized therapy to the patient, wherein the patient has been identified as infected by pathogen according to any one of the methods described herein.

For example, for a bacterial infection (e.g., bacteremia), a therapy may include an antibiotic. In some instances, an antibiotic may be administered orally. In other instances, the antibiotic may be administered intravenously. Exemplary non-limiting antibiotics that may be used in the methods of the invention include but are not limited to, acrosoxacin, amifioxacin, amikacin, amoxycillin, ampicillin, aspoxicillin, azidocillin, azithromycin, aztreonam, balofloxacin, benzylpenicillin, biapenem, brodimoprim, cefaclor, cefadroxil, cefatrizine, cefcapene, cefdinir, cefetamet, ceftmetazole, cefoxitin, cefprozil, cefroxadine, ceftarolin, ceftazidime, ceftibuten, ceftobiprole, cefuroxime, cephalexin, cephalonium, cephaloridine, cephamandole, cephazolin, cephradine, chlorquinaldol, chlortetracycline, ciclacillin, cinoxacin, ciprofloxacin, clarithromycin, clavulanic acid, clindamycin, clofazimine, cloxacillin, colistin, danofloxacin, dapsone, daptomycin, demeclocycline, dicloxacillin, difloxacin, doripenem, doxycycline, enoxacin, enrofloxacin, erythromycin, fleroxacin, flomoxef, flucloxacillin, flumequine, fosfomycin, gentamycin, isoniazid, imipenem, kanamycin, levofloxacin, linezolid, mandelic acid, mecillinam, meropenem, metronidazole, minocycline, moxalactam, mupirocin, nadifloxacin, nafcillin, nalidixic acid, netilmycin, netromycin, nifuirtoinol, nitrofurantoin, nitroxoline, norfloxacin, ofloxacin, oxacillin, oxytetracycline, panipenem, pefloxacin, phenoxymethylpenicillin, pipemidic acid, piromidic acid, pivampicillin, pivmecillinam, polymixin-b, prulifloxacin, rufloxacin, sparfloxacin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, temafioxacin, tetracycline, tetroxoprim, tigecycline, tinidazole, tobramycin, tosufloxacin, trimethoprim, vancomycin, and pharmaceutically acceptable salts or esters thereof.

In some embodiments, a method of treatment may include administering a treatment to an asymptomatic patient, for example, based on the detection and/or identification of a pathogen present in a biological sample derived from the patient by the methods of the invention. In other embodiments, a method of treatment may include administering a treatment to a symptomatic patient based on the detection of identification of a pathogen present in a biological sample derived from the patient by the methods of the invention. In several embodiments, the biological sample may contain cells, cell debris, and/or nucleic acids (e.g., DNA or RNA (e.g., mRNA)) derived from both the host subject and a pathogen, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), CSF, SF, urine, BAL, or sputum (e.g., purulent sputum or bloody sputum). In some embodiments, the biological sample is blood (e.g., whole blood, a crude whole blood lysate, serum, or plasma) or a bloody fluid (e.g., wound exudate, phlegm, bile, and the like). In particular embodiments, the biological sample is whole blood. In other particular embodiments, the biological sample is a crude whole blood lysate.

In some embodiments, the treatment selected for a patient is based on the detection and/or identification of a pathogen by the methods of the invention. Appropriate treatments for different pathogen species are known in the art. In one example, if a Gram positive bacterium is detected in a biological sample derived from a patient, a method of treatment may involve administration of vancomycin. In another example, if a Gram negative bacterium is detected in a biological sample derived from a patient, a method of treatment may involve administration of pipercillin-tazobactam. In another example, in some embodiments, if an Acinetobacter spp. (e.g., Acinetobacter baumannii) is detected in a biological sample derived from a patient, a method of treatment may involve administration of colistin, meropenem, and/or gentamicin. In another example, in some embodiments, if a Klebsiella spp. (e.g., Klebsiella pneumoniae, Klebsiella oxytoca, Klebsiella aerogenes, or Klebsiella variicola) is detected in a biological sample derived from a patient, a method of treatment may involve administration of meropenem. In yet another example, in some embodiments, if a Pseudomonas spp. (e.g., Pseudomonas aeruginosa) is detected in a biological sample derived from a patient, a method of treatment may involve administration of pipercillin-tazobactam. In a further example, in some embodiments, if an Escherichia spp. (e.g., Escherichia coli) is detected in a biological sample derived from a patient, a method of treatment may involve administration of meropenem. In another example, in some embodiments, if an Enterococcus spp. (e.g., Enterococcus faecium) is detected in a biological sample derived from a patient, a method of treatment may involve administration of daptomycin.

Exemplary therapies for the treatment of carbapenem-resistant infections may include treatment with a core therapy, either as the sole therapeutic agent (monotherapy) or in combination with one or more adjunct drugs (combination therapy). Treatments can be delivered using any suitable administration route, for example, per os, by intramuscular injection, or intravenously by traditional infusion, prolonged infusion (e.g., over 4 hours), or continuous infusion.

In general, monotherapies or core therapies for carbapenem-resistant infections that can be used include but are not limited to high-dose meropenem or doripenem, polymyxin B, colistin, tigecycline, ceftazidime-avibactam, meropenem-vaborbactam, aztreonam, and fosfomycin. Exemplary adjunct drugs include one or more of aminoglycosides, colistin, tigecycline, fosfomycin, gentamicin, tobramycin, amikacin, plazomicin, rimfampin, meropenem, doripenem, ertapenem, and imipenem.

Exemplary monotherapies or core therapies for carbapenem-resistant infections of the bloodstream include high-dose meropenem or doripenem and polymyxin B Suitable exemplary adjunct drugs for carbapenem-resistant infections of the bloodstream include one or more of an aminoglycoside, tigecycline, fosfomycin, and rimfampin.

Exemplary monotherapies or core therapies for carbapenem-resistant infections of the lung include high-dose meropenem or doripenem and polymyxin B. Suitable exemplary adjunct drugs for carbapenem-resistant infections of the lung include one or more of an aminoglycoside, tigecycline, fosfomycin, and rimfampin.

Exemplary monotherapies or core therapies for carbapenem-resistant infections of the gastrointestinal and/or biliary tract include high-dose meropenem or doripenem, polymyxin B, and high-dose tigecycline. Suitable exemplary adjunct drugs for carbapenem-resistant infections of the gastrointestinal and/or biliary tract include one or both of fosfomycin and rimfampin.

Exemplary monotherapies or core therapies for carbapenem-resistant infections of the urinary tract include high-dose meropenem or doripenem, an aminoglycoside, and fosfomycin. Suitable exemplary adjunct drugs for carbapenem-resistant infections of the urinary tract include one or both of colistin and an aminoglycoside.

High-dose meropenam can be defined as, e.g., 2000 mg q8 h over 4 h by IV. High-dose doripenam can be defined as, e.g., 1000-2000 mg q8 h over 4 h by IV. Ertapenem can be administered at, e.g., 1000 mg q24 h by IV. Gentamicin can be administered at., e.g., 5-10 mg/kg daily dose by IV. Tobramycin can be administered at., e.g., 5-10 mg/kg daily dose by IV. Amikacin can be administered at., e.g., 10-15 mg/kg daily dose by IV. Tigecycline can be administered at., e.g., 100-200 mg loading dose, then 50 mg q12 h mg/kg daily dose by IV. Fosfomycin can be administered at, e.g., 3 grams once or every 2-3 days per orum or 1-16 g daily by IV. Colistin can be administered at, e.g., 5 mg colistin base activity (CBA) per kg followed by maintenance doses of, e.g., 2.5-3 mg/kg per day. Polymyxin B can be administered at, e.g., 2-2.5 mg/kg followed by maintenance doses of, e.g., 2.5-3 mg/kg per day.

In one example, the invention provides a method for identifying a patient infected with an antibiotic resistant bacterial pathogen, the method including: (a) providing a biological sample obtained from the subject; and (b) detecting the presence of an antibiotic resistance gene in the biological sample according to any of the methods described herein, wherein the presence of an antibiotic resistance gene in the biological sample obtained from the subject identifies the subject as one who may be infected with an antibiotic resistant bacterial pathogen. In some embodiments, the method further includes selecting an optimized anti-bacterial therapy for the patient based on the presence of the antibiotic resistance gene. In some embodiments, the method further includes administering the optimized anti-bacterial therapy to the patient. In some embodiments, the optimized anti-bacterial therapy includes one or more antibiotic agents. In some embodiments, the one or more antibiotic agents is selected from the group consisting of polymyxin B, colistin, tigecycline, ceftazidime-avibactam, meropenem-vaborbactam, aztreonam, and fosfomycin.

In some embodiments, the drug (e.g., the antibiotic agent) is administered as a monotherapy. In some embodiments, the drug (e.g., the antibiotic agent) is administered as a combination therapy. In some embodiments, the combination therapy includes one or more additional antibiotic agents selected from the group consisting of an aminoglycoside, colistin, tigecycline, fosfomycin, gentamicin, tobramycin, amikacin, plazomicin, rimfampin, meropenem, doripenem, ertapenem, and imipenem.

Any suitable administration route described herein or known in the art may be used. Administration may be local or systemic. In some embodiments, the administration is parenteral. In some embodiments, the drug (e.g., the antibiotic agent) is administered to the patient orally, intravenously, intramuscularly, intra-arterially, subcutaneously, or intraperitoneally.

### Systems and Cartridges

The invention provides systems for carrying out the methods of the invention. The invention features methods and systems that may involve one or more cartridge units to provide a convenient method for placing all of the assay reagents and consumables onto the system. The panels of the invention may be contained on one or more cartridge units (e.g., 1, 2, 3, 4, 5, 6, 7, 8, 9, 10, 11, 12, 13, 14, 15, 16, 17, 18, 19, or 20 cartridge unites). For example, the system may be customized to perform a specific function, or adapted to perform more than one function, e.g., via changeable cartridge units containing arrays of micro wells with customized magnetic particles contained therein. The system can include a replaceable and/or interchangeable cartridge containing an array of wells pre-loaded with magnetic particles, and designed for detection and/or concentration measurement of a particular analyte. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a magnetic assisted agglomeration unit, or an NMR unit). The cartridge unit itself can interface directly with manipulation stations as well as with, for example, one or more MR reader(s). The cartridge unit can be a modular cartridge having an inlet module that can be sterilized independent of the reagent module. The systems may include one or more NMR units, MAA units, cartridge units, and agitation units, as described in WO 2012/054639. Any of the systems described in WO 2012/054639 may be used for embodiments that involve T2MR detection, e.g., for providing group-level information to focus or narrow subsequent sequencing. For example, FIG. 42 of WO 2012/054639 depicts a system that can be used for embodiments involving T2MR detection. In some embodiments, the system stores a sample containing one or more amplified target nucleic acids for downstream sequencing.

For example, in some embodiments, the systems include one or more sequencing units. In other embodiments, the system results in production of a sample that can be sequenced separately, for example, a sample that requires one or more further steps for sequencing (e.g., adaptor ligation and tagging). Such systems may further include other components for carrying out an automated assay of the invention, such as a thermocycling unit for the amplification of oligonucleotides; a centrifuge, a robotic arm for delivery an liquid sample from unit to unit within the system; one or more incubation units; a fluid transfer unit (i.e., pipetting device) for combining assay reagents and a biological sample (e.g., a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum) to form the liquid sample; a computer with a programmable processor for storing data, processing data, and for controlling the activation and deactivation of the various units according to a one or more preset protocols; and a cartridge insertion system for delivering pre-filled cartridges to the system, optionally with instructions to the computer identifying the reagents and protocol to be used in conjunction with the cartridge.

The sequencing unit may include any system or device that is known in the art for sequencing, e.g., massively parallel sequencing, long-read sequencing, or Sanger sequencing. Exemplary sequencing devices include but are not limited to ILLUMINA® systems (e.g., the ILLUMINA® iSeq 100 system, MiniSeq® system, MiSeq® systems, NextSeq® series platforms, HiSeq® series platforms, HiSeq X® series platforms, and NovaSeq® 6000 system); the BGISEQ-500 system; the 10× Genomics Chromium™ system; Ion Torrent sequencing systems (e.g., Ion PGM™, Ion Proton™, Ion S5™, and Ion S5 XL); Oxford Nanopore systems (e.g., MinION and PromethION); Pacific Biosystems systems (e.g., PacBio RS II or PacBio Sequel); and the Roche 454 system. Other sequencing systems are known in the art.

The systems of the invention can provide an effective means for high throughput detection and/or sequencing of analytes present in sample, e.g., an environmental sample or a biological sample from a subject. The detection methods may be used in a wide variety of circumstances including, without limitation, sequencing of nucleic acids, identification and/or quantification of analytes that are associated with specific biological processes, physiological conditions, disorders or stages of disorders. As such, the systems have a broad spectrum of utility in, for example, disease diagnosis, identification of drug resistance (e.g., antibiotic resistance), disease onset and recurrence, individual response to treatment versus population bases, monitoring of therapy, and antimicrobial stewardship. The devices and systems can provide a flexible system for personalized medicine. The system of the invention can be changed or interchanged along with a protocol or instructions to a programmable processor of the system to perform a wide variety of assays as described herein. The systems of the invention offer many advantages of a laboratory setting contained in a desk-top or smaller size automated instrument.

The invention provides methods and systems that may involve one or more cartridge units to provide a convenient method for placing all of the assay reagents (e.g., sequencing reagents) and consumables onto the system. For example, the cartridge units can include reagents for sequencing. Such reagents include, e.g., library preparation reagents (e.g., tagmentation reagents such as NEXTERA® XT library preparation reagents), buffers, adaptors, primers, enzymes (e.g., thermostable polymerases), and the like. The system can include a replaceable and/or interchangeable cartridge containing an array of wells pre-loaded, e.g., with sequencing reagents or magnetic particles, and designed for detection and/or sequencing of a particular analyte, e.g., a particular target nucleic acid. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a sequencing unit or an NMR unit). Any of the cartridges described in WO 2012/054639 can be used in the methods and systems described herein.

For example, provided herein is a system for the detection of one or more pathogen target nucleic acids, the system including: (a) a first unit including (i) a permanent magnet defining a magnetic field; (ii) a support defining a well holding a liquid sample including magnetic particles having a mean particle diameter between 600 and 1200 nm, preferably between 650 and 950 nm, and the one or more antibiotic resistance genes and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and (b) a second unit including a removable cartridge sized to facilitate insertion into and removal from the system, wherein the removable cartridge is a modular cartridge including (i) a reagent module for holding one or more assay reagents, (ii) a detection module including a detection chamber for holding a liquid sample including the magnetic particles and the one or more analytes, and, optionally, (iii) a sterilizable inlet module, wherein the reagent module, the detection module, and, optionally, the sterilizable inlet module, can be assembled into the modular cartridge prior to use, and wherein the detection chamber is removable from the modular cartridge, preferably, wherein the system further includes a system computer with processor for implementing an assay protocol and storing assay data, and wherein the removable cartridge further includes (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label including instructions for the programmable processor.

A modular cartridge can provide a simple means for cross contamination control during certain assays, including but not limited to distribution of amplification (e.g., PCR) products into multiple detection or sequencing aliquots. In addition, a modular cartridge can be compatible with automated fluid dispensing, and provides a way to hold reagents at very small volumes for long periods of time (in excess of a year). Finally, pre-dispensing these reagents allows concentration and volumetric accuracy to be set by the manufacturing process and provides for a point of care use instrument that is more convenient as it can require much less precise pipetting.

The modular cartridge can be designed for a multiplexed assay. The challenge in multiplexing assays is combining multiple assays which have incompatible assay requirements (i.e., different incubation times and/or temperatures) on one cartridge. The cartridge format depicted in FIGS. 14A-14C of WO 2012/054639 allows for the combination of different assays with dramatically different assay requirements. The cartridge features two main components: (i) a reagent module (i.e., the reagent strip portion) that contains all of the individual reagents required for the full assay panel (for example, a panel as described below), and (ii) the detection module. In some embodiments, a cartridge may be configured to detect and/or sequence from 2 to 24 or more target nucleic acids (e.g., 2, 3, 4, 5, 6, 7, 8, 9, 10, 11, 12, 13, 14, 15, 16, 17, 18, 19, 20, 21, 22, 23, 24, or more target nucleic acids), such as drug (e.g., antibiotic) resistance genes. In some embodiments, a cartridge may be configured to detect and/or sequence target nucleic acids from 2 to 24 or more pathogens (e.g., 2, 3, 4, 5, 6, 7, 8, 9, 10, 11, 12, 13, 14, 15, 16, 17, 18, 19, 20, 21, 22, 23, 24, or more pathogens). The detection modules contain only the parts of the cartridge that carry through the incubation, and can carry single assays or several assays, as needed.

The cartridge units can further include one or more populations of magnetic particles, either as a liquid suspension or dried magnetic particles which are reconstituted prior to use. For example, the cartridge units of the invention can include a compartment including from 1×10⁶ to 1×10¹³ magnetic particles (e.g., from 1×10⁶ to 1×10⁸, 1×10⁷ to 1×10⁹, 1×10⁸ to 1×10¹⁰, 1×10⁹ to 1×10¹¹, 1×10¹⁰ to 1×10¹², 1×10¹¹ to 1×10¹³, or from 1×10⁷ to 5×10⁸ magnetic particles) for assaying a single liquid sample.

The number of cartridges utilized can be scaled based on the number of analytes being detected. For example, the targets may be detected across 1, 2, 3, 4, 5, 6, 7, 8, 9, 10, 11, 12, or more cartridges. In some examples, each subpanel is included on a separate cartridge.

For example, provided herein is a removable cartridge comprising a well comprising any of the magnetic particles described herein. In some embodiments, the well includes a magnetic particle conjugated to one or more nucleic acid probes comprising a nucleic acid sequence set forth in any one of Tables 2, 4, 6, 8, 9, 11, 13, 15, 17, or 19 or a nucleic acid sequence having at least 95% sequence identity to any one of the nucleic acid sequences set forth in any one of Tables 2, 4, 6, 8, 9, 11, 13, 15, 17, or 19. In some embodiments, the removable cartridge further includes one or more chambers for holding a plurality of reagent modules for holding one or more assay reagents. In some embodiments, the removable cartridge further includes a chamber comprising beads for lysing cells. In some embodiments, the removable cartridge further includes a chamber comprising a polymerase. In some embodiments, the removable cartridge further includes a chamber comprising one or more primers. In some embodiments, the one or more primers comprising a nucleic acid sequence set forth in any one of Tables 1, 3, 5, 7, 10, 12, 14, 16, or 18, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence set forth in any one of Tables 1, 3, 5, 7, 10, 12, 14, 16, or 18.

### Magnetic Particles and T2MR

In some embodiments, the methods and systems of the invention may involve use of magnetic particles and NMR (e.g., T2MR). For example, T2MR can be used, for example, to rapidly and sensitively detect a target nucleic acid (e.g., a pathogen target nucleic acid, including an antibiotic resistance gene) and/or to obtain group-level information regarding a target nucleic acid, which can be used to narrow or focus sequencing analysis. The magnetic particles can be coated with a binding moiety (e.g., oligonucleotide, antibody, and the like) such that in the presence of analyte, or multivalent binding agent, aggregates are formed. Aggregation depletes portions of the sample from the microscopic magnetic non-uniformities that disrupt the solvent's T₂ signal, leading to an increase in T₂ relaxation (see, e.g., FIG. 3 of International Patent Application Publication No. WO 2012/054639, which is incorporated herein by reference in its entirety). Any NMR-based detection approach described in WO 2012/054639 may be used in the methods and systems described herein.

The T₂ measurement is a single measure of all spins in the ensemble, measurements lasting typically 1-10 seconds, which allows the solvent to travel hundreds of microns, a long distance relative to the microscopic non-uniformities in the liquid sample. Each solvent molecule samples a volume in the liquid sample and the T₂ signal is an average (net total signal) of all (nuclear spins) on solvent molecules in the sample; in other words, the T₂ measurement is a net measurement of the entire environment experienced by a solvent molecule, and is an average measurement of all microscopic non-uniformities in the sample.

The observed T₂ relaxation rate for the solvent molecules in the liquid sample is dominated by the magnetic particles, which in the presence of a magnetic field form high magnetic dipole moments. In the absence of magnetic particles, the observed T₂ relaxation rates for a liquid sample are typically long (i.e., T₂ (water)=approximately 2000 ms, T₂ (blood)=approximately 1500 ms). As particle concentration increases, the microscopic non-uniformities in the sample increase and the diffusion of solvent through these microscopic non-uniformities leads to an increase in spin decoherence and a decrease in the T₂ value. The observed T₂ value depends upon the particle concentration in a non-linear fashion, and on the relaxivity per particle parameter.

In embodiments that involve NMR detection, e.g., to provide rapid and sensitive detection and/or to obtain initial group-level information, the number of magnetic particles, and if present the number of agglomerant particles, remain constant during the assay. The spatial distribution of the particles changes when the particles cluster. Aggregation changes the average “experience” of a solvent molecule because particle localization into clusters is promoted rather than more even particle distributions. At a high degree of aggregation, many solvent molecules do not experience microscopic non-uniformities created by magnetic particles and the T₂ approaches that of solvent. As the fraction of aggregated magnetic particles increases in a liquid sample, the observed T₂ is the average of the non-uniform suspension of aggregated and single (unaggregated) magnetic particles. The assays of the invention are designed to maximize the change in T₂ with aggregation to increase the sensitivity of the assay to the presence of analytes, and to differences in analyte concentration.

Provided herein are magnetic particles conjugated to one or more nucleic acid probes for detection of one or more pathogen target nucleic acids described herein (e.g., one or more pathogen target nucleic acids in a multiplexed amplification reaction, wherein the multiplexed amplification reaction is configured to amplify a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids), (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids), and/or (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids). In one example, a magnetic particle may be conjugated to one or more probes set forth in any one of Tables 2, 4, 6, 8, 9, 11, 13, 15, 17, and 19.

For example, provided herein is a magnetic particle conjugated to a nucleic acid probe, wherein the nucleic acid probe is specific for an antibiotic resistance gene selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M-2, CTX-M-8, CTX-M 14, and/or CTX-M 15), mefA, mefE, ermA, ermB, SHV, TEM, FKS, PDR1, and ERG11. In some embodiments, the magnetic particle further includes an additional nucleic acid probe, wherein the second nucleic acid probe is specific for a second antibiotic resistance gene selected from the group consisting of NDM, KPC, IMP, VIM, OXA (e.g., OXA-23-like or OXA-48-like), DHA, CMY, FOX, mecA, mecC, MCR, vanA, vanB, CTX-M 14, CTX-M 15, mefA, mefE, ermA, ermB, SHV, TEM, FKS, PDR1, and ERG11. In some embodiments, the magnetic particle includes a first nucleic acid probe specific for DHA, and a second nucleic acid probe specific for CMY.

For example, the magnetic particles may be conjugated to one or more nucleic acid probes for detection of a universal CTX-M amplicon. In some embodiments, provided herein is a magnetic particle or a population of magnetic particles conjugated to a plurality of probes comprising (i) a plurality of 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a plurality of 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291) are used for detection of the universal CTX-M amplicon. In some embodiments, provided herein is a first population of magnetic particles conjugated to 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a second population of magnetic particles conjugated to 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291).

In some embodiments, the methods of the invention involve contacting a solution (e.g., a sample, e.g., a liquid sample, that includes whole blood or a crude whole blood lysate) with between from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter).

In some embodiments, the magnetic particles used in the methods and systems of the invention have a mean diameter of from 150 nm to 1200 nm (e.g., from 150 to 250, 200 to 350, 250 to 450, 300 to 500, 450 to 650, 500 to 700 nm, 700 to 850, 800 to 950, 900 to 1050, or from 1000 to 1200 nm). For example, in some embodiments, the magnetic particles used in the methods of the invention may have a mean diameter of from 150 nm to 699 nm (e.g., from 150 to 250, 200 to 350, 250 to 450, 300 to 500, 450 to 650, or from 500 to 699 nm). In other embodiments, the magnetic particles used in the methods of the invention may have a mean diameter of from 700 nm to 1200 nm (e.g., from 650 to 850, 650 to 950, 650 to 1050, 650 to 1200, 700 to 850, 800 to 950, 900 to 1050, or from 1000 to 1200 nm). In particular embodiments, the magnetic particles may have a mean diameter of from 700 nm to 950 nm (e.g., from 700 to 750, 700 to 800, 700 to 850, or from 700 to 900 nm).

In some embodiments, the magnetic particles used in the methods of the invention may have a T₂ relaxivity per particle of from 1×10⁸ to 1×10¹² mM⁻¹s⁻¹ (e.g., from 1×10⁸ to 1×10⁹, 1×10⁸ to 1×10¹⁰, 1×10⁹ to 1×10¹⁰, 1×10⁹ to 1×10¹¹, or from 1×10¹⁰ to 1×10¹² mM⁻¹s⁻¹). In some embodiments, the magnetic particles have a T₂ relaxivity per particle of from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹).

In some embodiments, the magnetic particles may be substantially monodisperse. In some embodiments, the magnetic particles in a liquid sample (e.g., a biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, BAL, or sputum) may exhibit nonspecific reversibility in the absence of the one or more analytes and/or multivalent binding agent. In some embodiments, the magnetic particles may further include a surface decorated with a blocking agent selected from albumin, fish skin gelatin, gamma globulin, lysozyme, casein, peptidase, and an amine-bearing moiety (e.g., amino polyethyleneglycol, glycine, ethylenediamine, or amino dextran.

The above methods can be used with any of the following categories of detection of aggregation or disaggregation described herein, including those described in WO 2012/054639, e.g., at pages 110-111.

### Primers and Probes

The invention provides primers and probes for use in the methods, panels, systems, cartridges, and kits provided herein. Any suitable primers and/or probes may be used in the context of the invention. Exemplary primers and probes that may be used in the context of the invention are set forth below.

In one example, provided herein is a nucleic acid primer comprising a nucleic acid sequence set forth in any one of Tables 1, 3, 5, 7, 10, 12, 14, 16, 18, or 26, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence set forth in any one of Tables 1, 3, 5, 7, 10, 12, 14, 16, 18, or 26. In one example, provided herein is a nucleic acid primer comprising a nucleic acid sequence set forth in any one of Tables 1, 3, 5, 7, 10, 12, 14, 16, or 18, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence set forth in any one of Tables 1, 3, 5, 7, 10, 12, 14, 16, or 18.

In another example, provided herein is a nucleic acid probe comprising a nucleic acid sequence set forth in any one of Tables 2, 4, 6, 8, 9, 11, 13, 15, 17, 19, or 26 or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence set forth in any one of Tables 2, 4, 6, 8, 9, 11, 13, 15, 17, 19, or 26. For example, provided herein is a nucleic acid probe comprising a nucleic acid sequence set forth in any one of Tables 2, 4, 6, 8, 9, 11, 13, 15, 17, or 19, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence set forth in any one of Tables 2, 4, 6, 8, 9, 11, 13, 15, 17, or 19. Any of the foregoing probes can be conjugated to one or more populations of magnetic particles.

Any of the primer and/or probe sequences described in International Patent Publication Nos. WO2012/054639, WO 2016/118766, WO 2017/127731, or WO 2018/213641, each of which is incorporated herein by reference in its entirety, may be used in the methods, panels, systems, cartridges, and kits provided herein.

Any of the primers and/or probes shown in Tables 1 and 2 can be used for amplifying a target nucleic acid characteristic of bacteria, e.g., for pan-level detection. The primer pair shown in Table 1 amplifies a large portion of bacteria, and the probes shown in Table 2 differentiate Gram positive bacteria from Gram negative bacteria. For example, in some embodiments, a target nucleic acid characteristic of bacteria (e.g., Gram positive and Gram negative bacteria) may be amplified in the presence of a forward primer that includes the oligonucleotide sequence CTCCTACGGGAGGCAGCAGT (SEQ ID NO: 173) and a reverse primer that includes the oligonucleotide sequence GTATTACCGCGGCTGCTGGCA (SEQ ID NO: 174). In some embodiments, an amplicon produced using these primers is detected by hybridization using a 5′ capture probe that includes the oligonucleotide sequence CTGACGGAGCAACGCCGCGTGAGTGA (SEQ ID NO: 175) and/or a 3′ capture probe that includes the oligonucleotide sequence CTAACCAGAAAGCCACGGCTAACTACG (SEQ ID NO: 176) to detect the presence of a Gram positive bacterium in a biological sample. In other embodiments, an amplicon produced using these primers is detected by hybridization using a 5′ capture probe that includes the oligonucleotide sequence CTGATCCAGCCATGCCGCGTGTATGA (SEQ ID NO: 177) and/or a 3′ capture probe that includes the oligonucleotide sequence CCGCAGAAGAAGCACCGGCTAACTCCG (SEQ ID NO: 178) to detect the presence of a Gram negative bacterium in a biological sample. In some embodiments, the 5′ capture probe and/or the 3′ capture probe is conjugated to a magnetic nanoparticle.

TABLE 1
Primer Sequences for Amplification/Detection of
Bacteria (PanGram)

| SEQ ID | | |
|---|---|---|
| NO | Name | Sequence |
| 173 | PanGram | CTCCTACGGGAGGCAGCAGT |
| | Forward Primer | |
| 174 | PanGram | GTATTACCGCGGCTGCTGGCA |
| | Reverse Primer | |

TABLE 2
Probe Sequences for Detection of Bacteria (PanGram)

| SEQ ID | | |
|---|---|---|
| NO | Name | Sequence |
| 175 | Gram Positive 5’ Capture | CTGACGGAGCAACGCCGCGTGAGTGA |
| | Probe | |
| 176 | Gram Positive 3’ Capture | CTAACCAGAAAGCCACGGCTAACTACG |
| | Probe | |
| 177 | Gram Negative 5’ | CTGATCCAGCCATGCCGCGTGTATGA |
| | Capture Probe | |
| 178 | Gram Negative 3’ | CCGCAGAAGAAGCACCGGCTAACTCCG |
| | Capture Probe | |

In one example, for amplification and/or detection of bacteria, any of the primers and/or probes set forth in Tables 3 and 4 may be used for amplification and/or detection of the indicated bacterial pathogens.

TABLE 3
Exemplary Primer Sequences for Bacterial Pathogens

| SEQ ID | | |
|---|---|---|
| NO | Name | Sequence |
| 81 | Acinetobacter baumannii | CGTTTTCCAAATCTGTAACAGACTGGG |
| | Forward Primer #1 | |
| 149 | Acinetobacter baumannii | GGAAGGGATCAGGTGGTTCACTCTT |
| | Forward Primer #2 | |
| 82 | Acinetobacter baumannii | AGGACGTTGATAGGTTGGATGTGGA |
| | Reverse Primer #1 | |
| 83 | Enterococcus spp. (e.g., | GGTAGCTATGTAGGGAAGGGATAAACGCTGA |
| | E. faecium and E. | |
| | faecalis) Forward Primer | |
| 84 | Enterococcus spp. (e.g., | GCGCTAAGGAGCTTAACTTCTGTGTTCG |
| | E. faecium and E. | |
| | faecalis) Reverse Primer | |
| 85 | Klebsiella pneumoniae | GACGGTTGTCCCGGTTTAAGCA |
| | Forward Primer | |
| 86 | Klebsiella pneumoniae | GCTGGTATCTTCGACTGGTCT |
| | Reverse Primer | |
| 87 | Pseudomonas | AGGCTGGGTGTGTAAGCGTTGT |
| | aeruginosa Forward | |
| | Primer | |
| 88 | Pseudomonas | CAAGCAATTCGGTTGGATATCCGTT |
| | aeruginosa Reverse | |
| | Primer | |
| 89 | Staphylococcus aureus | GGTAATGAATTACCT/i6diPr/TCTCTGCTGGTTTCTTCTT |
| | femA Forward Primer | |
| 90 | Staphylococcus aureus | ACCAGCATCTTC/i6diPr/GCATCTTCTGTAAA |
| | femA Reverse Primer | |
| 91 | Staphylococcus aureus | GAAGTTATGTTT/i6diPr/CTATTCGAATCGTGGTCCAGT |
| | femB Forward Primer | |
| 92 | Staphylococcus aureus | GTTGTAAAGCCATGATGCTCGTAACCA |
| | femB Reverse Primer | |
| 132 | Escherichia coli Forward | GCATTAATCGACGGTATGGTTGACC |
| | Primer | |
| 133 | Escherichia coli Reverse | CCTGCTGAAACAGGTTTTCCCACATA |

Primer

TABLE 4
Exemplary Probe Sequences for Bacterial Pathogens

| SEQ ID | | |
|---|---|---|
| NO | Name | Sequence |
| 95 | Acinetobacter baumannii | TGAGGCTTGACTATACAACACC |
| | 5’ Capture Probe | |
| 96 | Acinetobacter baumannii | CTAAAATGAACAGATAAAGTAAGATTCAA |
| | 3’ Capture Probe | |
| 99 | E. faecium 5’ Capture | AAAACTTATATGACTTCAAATCCAGTTTT |
| | Probe #1 | |
| 150 | E. faecium 5’ Capture | AAAACTTATGTGACTTCAAATCCAGTTTT |
| | Probe #2 | |
| 100 | E. faecium 3’ Capture | TTTACTCAATAAAAGATAACACCACAG |
| | Probe #1 | |
| 151 | E. faecium 3’ Capture | TTTACTCAATAAAAGATAACACCACAGT |
| | Probe #2 | |
| 103 | E. faecalis 5’ Capture | TGGATAAGTAAAAGCAACTTGGTT |
| | Probe | |
| 104 | E. faecalis 3’ Capture | AATGAAGATTCAACTCAATAAGAAACAACA |
| | Probe | |
| 107 | Klebsiella pneumoniae 5’ | TACCAAGGCGCTTGAGAGAACTC |
| | Capture Probe | |
| 108 | Klebsiella pneumoniae 3’ | CTGGTGTGTAGGTGAAGTC |
| | Capture Probe | |
| 111 | Pseudomonas | GTGTGTTGTAGGGTGAAGTCGAC |
| | aeruginosa 5’ Capture | |
| | Probe #1 | |
| 153 | Pseudomonas | TCTGACGATTGTGTGTTGTAAGG |
| | aeruginosa 5’ Capture | |
| | Probe #2 | |
| 112 | Pseudomonas | CACCTTGAAATCACATACCTGA |
| | aeruginosa 3’ Capture | |
| | Probe #1 | |
| 154 | Pseudomonas | GGATAGACGTAAGCCCAAGC |
| | aeruginosa 3’ Capture | |
| | Probe #2 | |
| 115 | Staphylococcus aureus | CCATTTGAAGTTGTTTATTATGC |
| | femA 5’ Capture Probe | |
| 116 | Staphylococcus aureus | GGGAAATGATTAATTATGCATTAAATC |
| | femA 3’ Capture Probe | |
| 119 | Staphylococcus aureus | TTTTTCAGATTTAGGATTAGTTGATT |
| | femB 5’ Capture Probe | |
| 120 | Staphylococcus aureus | GATCCGTATTGGTTATATCATC |
| | femB 3’ Capture Probe | |
| 134 | Escherichia coli 5’ | AGTGATGATGAGTTGTTTGCCAGTG |
| | Capture Probe | |
| 135 | Escherichia coli 3’ | TGAATTGTCGCCGCGTGACCAG |
| | Capture Probe | |

For example, in some embodiments, an Acinetobacter baumannii target nucleic acid is derived from a region that includes parts or all of the internally transcribed sequence (ITS) between the 5S and 23S rRNA genes (i.e., the ITS2 region). In particular embodiments, an Acinetobacter baumannii target nucleic acid may be amplified in the presence of a forward primer that includes the oligonucleotide sequence CGT TTT CCA AAT CTG TAA CAG ACT GGG (SEQ ID NO: 81) or GGA AGG GAT CAG GTG GTT CAC TCT T (SEQ ID NO: 149) and a reverse primer that includes the oligonucleotide sequence AGG ACG TTG ATA GG TTG GAT GTG GA (SEQ ID NO: 82). For example, in particular embodiments, an Acinetobacter baumannii target nucleic acid may be amplified in the presence of a forward primer that includes the oligonucleotide sequence GGA AGG GAT CAG GTG GTT CAC TCT T (SEQ ID NO: 149) and a reverse primer that includes the oligonucleotide sequence AGG ACG TTG ATA GG TTG GAT GTG GA (SEQ ID NO: 82). In some embodiments, an amplicon produced using these primers is detected by hybridization using a 5′ capture probe that includes the oligonucleotide sequence TGA GGC TTG ACT ATA CAA CAC C (SEQ ID NO: 95) and/or a 3′ capture probe that includes the oligonucleotide sequence CTA AAA TGA ACA GAT AAA GTA AGA TTC AA (SEQ ID NO: 96) to detect the presence of Acinetobacter baumannii in a biological sample. In some embodiments, an amplicon produced using these primers is detected by hybridization using a 5′ capture probe that includes the oligonucleotide sequence of SEQ ID NO: 97 and/or a 3′ capture probe that includes the oligonucleotide sequence of SEQ ID NO: 98 to detect the presence of Acinetobacter baumannii in a biological sample. In some embodiments, the 5′ capture probe and/or the 3′ capture probe is conjugated to a magnetic nanoparticle. In some embodiments, a control target nucleic acid for A. baumannii may comprise the nucleic acid sequence of SEQ ID NO: 125.

In some embodiments, an Enterococcus spp. target nucleic acid is derived from a region that includes parts or all of the ITS between the 23S and 5S rRNA genes. In particular embodiments, an target nucleic acid that is specific for Enterococcus faecium and Enterococcus faecalis may be amplified in the presence of a forward primer that includes the oligonucleotide sequence GGT AGC TAT GTA GGG AAG GGA TAA ACG CTG A (SEQ ID NO: 83) and a reverse primer that includes the oligonucleotide sequence GCG CTA AGG AGC TTA ACT TCT GTG TTC G (SEQ ID NO: 84). In some embodiments, an amplicon produced using these primers is detected by hybridization using a 5′ capture probe that includes the oligonucleotide sequence AAA ACT TAT ATG ACT TCA AAT CCA GTT TT (SEQ ID NO: 99) or AAA ACT TAT GTG ACT TCA AAT CCA GTT TT (SEQ ID NO: 150) and/or a 3′ capture probe that includes the oligonucleotide sequence TTT ACT CAA TAA AAG ATA ACA CCA CAG (SEQ ID NO: 100) or TTT ACT CAA TAA AAG ATA ACA CCA CAG T (SEQ ID NO: 151) to detect the presence of Enterococcus faecium in a biological sample. In particular embodiments, an amplicon produced using these primers is detected by hybridization using a 5′ capture probe that includes the oligonucleotide sequence AAA ACT TAT GTG ACT TCA AAT CCA GTT TT (SEQ ID NO: 150) and/or a 3′ capture probe that includes the oligonucleotide sequence TTT ACT CAA TAA AAG ATA ACA CCA CAG T (SEQ ID NO: 151) to detect the presence of Enterococcus faecium in a biological sample. In some embodiments, an amplicon produced using these primers is detected by hybridization using a 5′ capture probe that includes the oligonucleotide sequence of SEQ ID NO: 101 and/or a 3′ capture probe that includes the oligonucleotide sequence of SEQ ID NO: 102 to detect the presence of Enterococcus faecium in a biological sample. In some embodiments, an amplicon produced using these primers is detected by hybridization using a 5′ capture probe that includes the oligonucleotide sequence TGG ATA AGT AAA AGC AAC TTG GTT (SEQ ID NO: 103) and/or a 3′ capture probe that includes the oligonucleotide sequence AAT GAA GAT TCA ACT CAA TAA GAA ACA ACA (SEQ ID NO: 104) to detect the presence of Enterococcus faecalis in a biological sample. In some embodiments, an amplicon produced using these primers is detected by hybridization using a 5′ capture probe that includes the oligonucleotide sequence of SEQ ID NO: 105 and/or a 3′ capture probe that includes the oligonucleotide sequence of SEQ ID NO: 106 to detect the presence of Enterococcus faecalis in a biological sample. In some embodiments, the 5′ capture probe and/or the 3′ capture probe is conjugated to a magnetic nanoparticle. In some embodiments, a control target nucleic acid for Enterococcus faecium may comprise the nucleic acid sequence of SEQ ID NO: 126. In other embodiments, a control target nucleic acid for Enterococcus faecium may comprise the nucleic acid sequence of SEQ ID NO: 157. In some embodiments, a control target nucleic acid for Enterococcus faecalis may comprise the nucleic acid sequence of SEQ ID NO: 127.

In some embodiments, a Klebsiella pneumoniae target nucleic acid is derived from a 23S rRNA gene. In particular embodiments, a Klebsiella pneumoniae target nucleic acid may be amplified in the presence of a forward primer that includes the oligonucleotide sequence GAC GGT TGT CCC GGT TTA AGC A (SEQ ID NO: 85) and a reverse primer that includes the oligonucleotide sequence GCT GGT ATC TTC GAC TGG TCT (SEQ ID NO: 86). In some embodiments, an amplicon produced using these primers is detected by hybridization using a 5′ capture probe that includes the oligonucleotide sequence TAC CAA GGC GCT TGA GAG AAC TC (SEQ ID NO: 107) and/or a 3′ capture probe that includes the oligonucleotide sequence CTG GTG TGT AGG TGA AGT C (SEQ ID NO: 108) to detect the presence of Klebsiella pneumoniae in a biological sample. In some embodiments, an amplicon produced using these primers is detected by hybridization using a 5′ capture probe that includes the oligonucleotide sequence of SEQ ID NO: 109 and/or a 3′ capture probe that includes the oligonucleotide sequence of SEQ ID NO: 110 to detect the presence of Klebsiella pneumoniae in a biological sample. In some embodiments, the 5′ capture probe and/or the 3′ capture probe is conjugated to a magnetic nanoparticle. In some embodiments, a control target nucleic acid for Klebsiella pneumoniae may comprise the nucleic acid sequence of SEQ ID NO: 128.

In some embodiments, a Pseudomonas aeruginosa target nucleic acid is derived from a region that includes parts or all of the ITS between the 23S and 5S rRNA genes. In particular embodiments, a Pseudomonas aeruginosa target nucleic acid may be amplified in the presence of a forward primer that includes the oligonucleotide sequence AGG CTG GGT GTG TAA GCG TTG T (SEQ ID NO: 87) and a reverse primer that includes the oligonucleotide sequence CAA GCA ATT CGG TTG GAT ATC CGT T (SEQ ID NO: 88). In some embodiments, an amplicon produced using these primers is detected by hybridization using a 5′ capture probe that includes the oligonucleotide sequence GTG TGT TGT AGG GTG AAG TCG AC (SEQ ID NO: 111) or TCT GAC GAT TGT GTG TTG TAA GG (SEQ ID NO: 153) and/or a 3′ capture probe that includes the oligonucleotide sequence CAC CTT GAA ATC ACA TAC CTG A (SEQ ID NO: 112) or GGA TAG ACG TAA GCC CAA GC (SEQ ID NO: 154) to detect the presence of Pseudomonas aeruginosa in a biological sample. In particular embodiments, an amplicon produced using these primers is detected by hybridization using a 5′ capture probe that includes the oligonucleotide sequence TCT GAC GAT TGT GTG TTG TAA GG (SEQ ID NO: 153) and/or a 3′ capture probe that includes the oligonucleotide GGA TAG ACG TAA GCC CAA GC (SEQ ID NO: 154) to detect the presence of Pseudomonas aeruginosa in a biological sample. In some embodiments, an amplicon produced using these primers is detected by hybridization using a 5′ capture probe that includes the oligonucleotide sequence of SEQ ID NO: 113 and/or a 3′ capture probe that includes the oligonucleotide sequence of SEQ ID NO: 114 to detect the presence of Pseudomonas aeruginosa in a biological sample. In some embodiments, the 5′ capture probe and/or the 3′ capture probe is conjugated to a magnetic nanoparticle. In some embodiments, a control target nucleic acid for Pseudomonas aeruginosa may comprise the nucleic acid sequence of SEQ ID NO: 129.

In some embodiments, a Staphylococcus aureus target nucleic acid is derived from the femAB operon. The femAB operon codes for two nearly identical approximately 50 kDa proteins involved in the formation of the Staphylococcal pentaglycine interpeptide bridge in peptidoglycan. These chromosomally-encoded proteins are considered as factors that influence the level of methicillin resistance and as essential housekeeping genes. femB is one gene in the femA/B operon, also referred to as graR, the two component response regulator of methicillin resistance. femB encodes an aminoacyltransferase, whereas femA encodes a regulatory factor that is essential for expression of femB and therefore methicillin resistance expression. In some embodiments, a Staphylococcus aureus target nucleic acid is derived from the femA gene. For example, in particular embodiments, a Staphylococcus aureus target nucleic acid may be amplified in the presence of a forward primer that includes the oligonucleotide sequence GGT AAT GAATTA CCT/i6diPr/TC TCT GCT GGTTTC TTC TT (SEQ ID NO: 89) and a reverse primer that includes the oligonucleotide sequence ACC AGC ATC TTC/i6diPr/GC ATC TTC TGT AAA (SEQ ID NO: 90). Note that “/i6diPr/” indicates 2,6-Diaminopurine. In some embodiments, an amplicon produced using these primers is detected by hybridization using a 5′ capture probe that includes the oligonucleotide sequence CCA TTT GAA GTT GTT TAT TAT GC (SEQ ID NO: 115) and/or a 3′ capture probe that includes the oligonucleotide sequence GGG AAA TGA TTA ATT ATG CAT TAA ATC (SEQ ID NO: 116) to detect the presence of Staphylococcus aureus in a biological sample. In some embodiments, an amplicon produced using these primers is detected by hybridization using a 5′ capture probe that includes the oligonucleotide sequence of SEQ ID NO: 117 and/or a 3′ capture probe that includes the oligonucleotide sequence of SEQ ID NO: 118 to detect the presence of Staphylococcus aureus in a biological sample. In some embodiments, the 5′ capture probe and/or the 3′ capture probe is conjugated to a magnetic nanoparticle.

In some embodiments, a Staphylococcus aureus target nucleic acid is derived from the femB gene. For example, in other particular embodiments, a Staphylococcus aureus target nucleic acid may be amplified in the presence of a forward primer that includes the oligonucleotide sequence GAA GTT ATG TTT/i6diPr/CT ATT CGA ATC GTG GTC CAGT (SEQ ID NO: 91) and a reverse primer that includes the oligonucleotide sequence GTT GTA AAG CCA TGA TGC TCG TAA CCA (SEQ ID NO: 92). In some embodiments, an amplicon produced using these primers is detected by hybridization using a 5′ capture probe that includes the oligonucleotide sequence TT TTT CAG ATT TAG GAT TAG TTG ATT (SEQ ID NO: 119) and/or a 3′ capture probe that includes the oligonucleotide sequence GAT CCG TAT TGG TTA TAT CAT C (SEQ ID NO: 120) to detect the presence of Staphylococcus aureus in a biological sample. In some embodiments, an amplicon produced using these primers is detected by hybridization using a 5′ capture probe that includes the oligonucleotide sequence of SEQ ID NO: 121 and/or a 3′ capture probe that includes the oligonucleotide sequence of SEQ ID NO: 122 to detect the presence of Staphylococcus aureus in a biological sample. In some embodiments, the 5′ capture probe and/or the 3′ capture probe is conjugated to a magnetic nanoparticle. In some embodiments, a Staphylococcus aureus target nucleic acid includes all or a portion of both the femA gene and the femB gene. In some embodiments, a control target nucleic acid for Staphylococcus aureus femA may comprise the nucleic acid sequence of SEQ ID NO: 130. In some embodiments, a control target nucleic acid for Staphylococcus aureus femB may comprise the nucleic acid sequence of SEQ ID NO: 131.

In particular embodiments, an Escherichia coli target nucleic acid is derived from the yfcL gene. The yfcL gene is within an E. coli-specific Chaperone-Usher Fimbriae gene cluster (see, e.g., Wurpel et al. PLoS One Vol 8, e52835, 2013). For example, in other particular embodiments, Escherichia coli yfcL may be amplified in the presence of a forward primer that includes the oligonucleotide sequence GCA TTA ATC GAC GGT ATG GTT GAC C (SEQ ID NO: 132) and a reverse primer that includes the oligonucleotide sequence CCT GCT GAA ACA GGT TTT CCC ACA TA (SEQ ID NO: 133). In some embodiments, an amplicon produced using these primers is detected by hybridization using a 5′ capture probe that includes the oligonucleotide sequence AGT GAT GAT GAG TTG TTT GCC AGT G (SEQ ID NO: 134) and/or a 3′ capture probe that includes the oligonucleotide sequence TGA ATT GTC GCC GCG TGA CCA G (SEQ ID NO: 135) to detect the presence of Escherichia coli in a biological sample. In some embodiments, the 5′ capture probe and/or the 3′ capture probe is conjugated to a magnetic nanoparticle.

Any of the primers and/or probes shown in Tables 5 and 6 can be used for amplifying a target nucleic acid characteristic of Staphylococcus spp., e.g., for genus-level detection. For example, in some embodiments, a target nucleic acid characteristic of Staphylococcus spp. may be amplified in the presence of a forward primer that includes the oligonucleotide sequence CACATTCTTTTATCACGTAACGTTGGTGT (SEQ ID NO: 179) and a reverse primer that includes the oligonucleotide sequence CCAGGCATTACCATTTCAGTACCTTCTGGTAA (SEQ ID NO: 180). In some embodiments, an amplicon produced using these primers is detected by hybridization using a 5′ capture probe that includes the oligonucleotide sequence CCAGTTACGTCAGTAGTACGGAA (SEQ ID NO: 181) and/or a 3′ capture probe that includes the oligonucleotide sequence TTTGATTTGACCACGTTCAACAC (SEQ ID NO: 182) to detect the presence of Staphylococcus spp. in a biological sample. In some embodiments, the 5′ capture probe and/or the 3′ capture probe is conjugated to a magnetic nanoparticle.

TABLE 5
Exemplary Primer Sequences for Staphylococcus spp.

| SEQ ID | | |
|---|---|---|
| NO | Name | Sequence |
| 179 | Staphylococcus spp. | CACATTCTTTTATCACGTAACGTTGGTGT |
| | Forward Primer | |
| 180 | Staphylococcus spp. | CCAGGCATTACCATTTCAGTACCTTCTGGTAA |
| | Reverse Primer | |

TABLE 6
Exemplary Probe Sequences for
Staphylococcus spp.

| SEQ ID | | |
|---|---|---|
| NO | Name | Sequence |
| 181 | Staphylococcus | CCAGTTACGTCA |
| | spp. 5′ | GTAGTACGGAA |
| | Capture Probe | |
| 182 | Staphylococcus | TTTGATTTGACC |
| | spp. 3′ | ACGTTCAACAC |
| | Capture Probe | |

Any of the primers and/or probes shown in Table 7, Table 8, and Table 9 can be used for amplifying and/or detecting a target nucleic acid characteristic of Candida spp., including for the indicated Candida species.

TABLE 7
Exemplary Primer Sequences for
Candida spp.

| SEQ ID | | |
|---|---|---|
| NO | Name | Sequence |
| 93 | Candida spp. | GGCATGCCTGTTTGAGCGTC |
| | Forward | |
| | Primer #1 | |
| 158 | Candida spp. | GGCATGCCTGTTTGAGCGT |
| | Forward | |
| | Primer #2 | |
| 159 | Candida spp. | GGGCATGCCTGTTTGAGCGT |
| | Forward | |
| | Primer #3 | |
| 94 | Candida spp. | GCTTATTGATATGCTTAAGT |
| | Reverse | TCAGCGGGT |
| | Primer | |

TABLE 8
Exemplary Probe Sequences for Candida spp.

| SEQ ID | | |
|---|---|---|
| NO | Name | Sequence |
| 136 | Candida albicans | ACCCAGCGGTTTGAGGGAGAAAC |
| | 5′ Capture Probe | |
| 137 | Candida albicans | AAAGTTTGAAGATATACGTGGTG |
| | 3′ Capture Probe | GACGTTA |
| 138 | Candida krusei | CGC ACG CGC AAG ATG GAA |
| | Capture Probe #1 | ACG |
| 139 | Candida krusei | AAG TTC AGC GGG TAT TCC |
| | Capture Probe #2 | TAC CT |
| 140 | Candida krusei | AGC TTT TTG TTG TCT CGC |
| | Capture Probe #3 | AAC ACT CGC |
| 141 | Candida glabrata | CTA CCA AAC ACA ATG TGT |
| | 5′ Capture Probe | TTG AGA AG |
| 142 | Candida glabrata | CCT GAT TTG AGG TCA AAC |
| | 3′ Capture Probe | TTA AAG ACG TCT G |
| 143 | C. parapsilosis/ | AGT CCT ACC TGA TTT GAG |
| | C. tropicalis | GTCNitlndAA |
| | Capture | |
| | Probe #1 | |
| 64 | C. parapsilosis/ | CCG NitlndGG GTT TGA |
| | C. tropicalis | GGG AGA AAT |
| | Capture | |
| | Probe #2 | |
| 144 | C. parapsilosis/ | AAA GTT ATG AAATAA ATT |
| | C. tropicalis | GTG GTG GCC ACT AGC |
| | Capture | |
| | Probe #3 | |
| 145 | C. parapsilosis/ | ACC CGG GGGTTT GAG GGA |
| | C. tropicalis | GAA A |
| | Capture | |
| | Probe #4 | |
| 146 | C. parapsilosis/ | AGT CCT ACC TGA TTT GAG |
| | C. tropicalis | GTC GAA |
| | Capture | |
| | Probe #5 | |
| 147 | C. parapsilosis/ | CCG AGG GTT TGA GGG AGA |
| | C. tropicalis | AAT |
| | Capture | |
| | Probe #6 | |

TABLE 9
Exemplary Probe Sequences for Candida auris
and other Candida spp.

| SEQ ID | | |
|---|---|---|
| NO | Name | Sequence |
| 160 | Candida auris 5′ | CTA CCT GAT TTG AGG |
| | Capture Probe | CGA CAA CAA AAC |
| 161 | Candida auris 3′ | CCG CGA AGA TTG GTG |
| | Capture Probe | AGA AGA CAT |
| 162 | Candida lusitaniae | CCT ACC TGA TTT GAG |
| | 5′ Capture Probe | GGC GAA ATG TC |
| 163 | Candida lusitaniae | GGA GCA ACG CCT AAC |
| | 3′ Capture Probe | CGG G |
| 164 | Candida haemulonii | GTC CTA CCT GAT TTG |
| | 5′ Capture Probe | AGG GGA AAA AGC |
| 165 | Candida haemulonii | AAC AAA TCC ACC AAC |
| | 3′ Capture Probe | GGT GAG AAG ATA T |
| 166 | Candida | CGT AGA CTT CGC TGC |
| | duobushaemulonii 5′ | GGA T |
| | Capture Probe #1 | |
| 167 | Candida | GCG TAG ACT TCG CTG |
| | duobushaemulonii 5′ | CGG AT |
| | Capture Probe #2 | |
| 168 | Candida | CTG GGC GGT GAG AAG |
| | duobushaemulonii 3′ | AAA TC |
| | Capture Probe | |
| 169 | Candida | GCG TAG ACT TCG CTG |
| | pseudohaemulonii 5′ | CTG GAA |
| | Capture Probe | |
| 170 | Candida | CCG TGC GGT GAG AAG |
| | pseudohaemulonii 3′ | AAA TC |
| | Capture Probe | |
| 171 | Candida | TCC TAC CTG ATT TGA |
| | duobushaemulonii/ | GGA AAT AGC ATG G |
| | Candida | |
| | pseudohaemulonii 5′ | |
| | Capture Probe | |
| 172 | Candida | ATT TAG CGG ATG CAA |
| | duobushaemulonii/ | AAC CAC C |
| | Candida | |
| | pseudohaemulonii 3′ | |
| | Capture Probe | |

Detection of a Candida species can be performed as described, for example, in International Patent Application Publication No. WO 2012/054639, which is incorporated herein by reference in its entirety. In particular embodiments, a Candida spp. target nucleic acid may be amplified in the presence of a forward primer that includes the oligonucleotide sequence GGC ATG CCT GTT TGA GCG TC (SEQ ID NO: 93) and a reverse primer that includes the oligonucleotide sequence GCT TAT TGA TAT GCT TAA GTT CAG CGG GT (SEQ ID NO: 94). In some embodiments, a Candida amplicon produced by amplification of a Candida target nucleic acid in the presence of a forward primer comprising the oligonucleotide sequence GGC ATG CCT GTT TGA GCG TC (SEQ ID NO: 93) and a reverse primer that includes the oligonucleotide sequence GCT TAT TGA TAT GCT TAA GTT CAG CGG GT (SEQ ID NO: 94) is detected by hybridization to a first nucleic acid probe and a second nucleic acid probe conjugated to one or more populations of magnetic particles. For example, certain embodiments, (i) the Candida species is Candida albicans, the first probe includes the oligonucleotide sequence ACC CAG CGG TTT GAG GGA GAA AC (SEQ ID NO: 136), and the second probe includes the oligonucleotide sequence AAA GTT TGA AGA TAT ACG TGG TGG ACG TTA (SEQ ID NO: 137); (ii) the Candida species is Candida krusei and the first probe and the second probe include an oligonucleotide sequence selected from: CGC ACG CGC AAG ATG GAA ACG (SEQ ID NO: 138), AAG TTC AGC GGG TAT TCC TAC CT (SEQ ID NO: 139), and AGC TTT TTG TTG TCT CGC AAC ACT CGC (SEQ ID NO: 140); (iii) the Candida species is Candida glabrata, the first probe includes the oligonucleotide sequence: CTA CCA AAC ACA ATG TGT TTG AGA AG (SEQ ID NO: 141), and the second probe includes the oligonucleotide sequence: CCT GAT TTG AGG TCA AAC TTA AAG ACG TCT G (SEQ ID NO: 142); and (iv) the Candida species is Candidaparapsi/osis or Candida tropicalis and the first probe and the second probe include an oligonucleotide sequence selected from: AGT CCT ACC TGA TTT GAG GTCNitIndAA (SEQ ID NO: 143), CCG NitIndGG GTT TGA GGG AGA AAT (SEQ ID NO: 64), AAA GTT ATG AAATAA ATT GTG GTG GCC ACT AGC (SEQ ID NO: 144), ACC CGG GGGTTT GAG GGA GAA A (SEQ ID NO: 145), AGT CCT ACC TGA TTT GAG GTC GAA (SEQ ID NO: 146), and CCG AGG GTT TGA GGG AGA AAT (SEQ ID NO: 147). In some embodiments, the first probe comprises the oligonucleotide sequence of SEQ ID NO: 123 and the second probe comprises the oligonucleotide sequence of SEQ ID NO: 124.

Detection of Candida auris, Candida lusitaniae, Candida haemulonii, Candida duobushaemulonii, and/or Candida pseudohaemulonii, or other species such as Candida lusitaniae can be performed as described in International Patent Application Publication No. WO 2018/213641, which is incorporated herein by reference in its entirety. For example, in some embodiments, a Candida spp. target nucleic acid may be amplified in the presence of a primer pair comprising a forward primer comprising the oligonucleotide sequence: GGC ATG CCT GTT TGA GCG T (SEQ ID NO: 158) or GGG CAT GCC TGT TTG AGC GT (SEQ ID NO: 159) and a reverse primer comprising the oligonucleotide sequence: GCT TAT TGA TAT GCT TAA GTT CAG CGG GT (SEQ ID NO: 94). In some embodiments, a Candida spp. amplicon produced by amplification of a Candida target nucleic acid in the presence of a primer pair comprising a forward primer comprising the oligonucleotide sequence: GGC ATG CCT GTT TGA GCG T (SEQ ID NO: 158) or GGG CAT GCC TGT TTG AGC GT (SEQ ID NO: 159) and a reverse primer comprising the oligonucleotide sequence: GCT TAT TGA TAT GCT TAA GTT CAG CGG GT (SEQ ID NO: 94) is detected by hybridization to a first nucleic acid probe and a second nucleic acid probe conjugated to one or more populations of magnetic particles. In some embodiments, the Candida species is Candida auris, and the first probe comprises the oligonucleotide sequence: CTA CCT GAT TTG AGG CGA CAA CAA AAC (SEQ ID NO: 160), and the second probe comprises the oligonucleotide sequence: CCG CGA AGA TTG GTG AGA AGA CAT (SEQ ID NO: 161). In some embodiments, the Candida species is Candida lusitaniae, and the first probe comprises the oligonucleotide sequence: CCT ACC TGA TTT GAG GGC GAA ATG TC (SEQ ID NO: 162), and the second probe comprises the oligonucleotide sequence: GGA GCA ACG CCT AAC CGG G (SEQ ID NO: 163). In some embodiments, the Candida species is Candida haemulonii, and the first probe comprises the oligonucleotide sequence: GTC CTA CCT GAT TTG AGG GGA AAA AGC (SEQ ID NO: 164), and the second probe comprises the oligonucleotide sequence: AAC AAA TCC ACC AAC GGT GAG AAG ATA T (SEQ ID NO: 165). In some embodiments, the Candida species is Candida duobushaemulonii, and the first probe comprises the oligonucleotide sequence: CGT AGA CTT CGC TGC GGA T (SEQ ID NO: 166) or GCG TAG ACT TCG CTG CGG AT (SEQ ID NO: 167), and the second probe comprises the oligonucleotide sequence: CTG GGC GGT GAG AAG AAA TC (SEQ ID NO: 168). In some embodiments, the Candida species is Candida pseudohaemulonii, and the first probe comprises the oligonucleotide sequence: GCG TAG ACT TCG CTG CTG GAA (SEQ ID NO: 169), and the second probe comprises the oligonucleotide sequence: CCG TGC GGT GAG AAG AAA TC (SEQ ID NO: 170). In some embodiments, the Candida species is Candida duobushaemulonii or Candida pseudohaemulonii, and the first probe comprises the oligonucleotide sequence: TCC TAC CTG ATT TGA GGA AAT AGC ATG G (SEQ ID NO: 171), and the second probe comprises the oligonucleotide sequence: ATT TAG CGG ATG CAA AAC CAC C (SEQ ID NO: 172).

Any of the primer and/or probe sequences set forth in Tables 10-17 can be used for amplification and/or detection of the indicated antibiotic resistance genes. For example, any of the antibiotic resistance gene target nucleic acids amplified by the primers set forth in Table 10 can be detected using the probes set forth in Table 11. In another example, any of the antibiotic resistance gene target nucleic acids amplified by the primers set forth in Table 12 can be detected using the probes set forth in Table 13. In some examples, any of the primers set forth in Table 14 may be used in place of any of the primers set forth in Table 12. In some examples, any of the probes set forth in Table 15 may be used in place of any of the probes set forth in Table 13.

TABLE 10
Exemplary Primer Sequences for
Antibiotic Resistance Genes

| SEQ ID | | |
|---|---|---|
| NO | Name | Sequence |
| 1. | NDM F Primer | CCAGCTCGCACCGAATGTCT |
| 2. | NDM R Primer | CATCTTGTCCTGATGCGCGTGA |
| | | GTCA |
| 3. | KPC F Primer | TTTCTGCCACCGCGCTGAC |
| 4. | KPC R Primer | GCAGCAAGAAAGCCCTTGAATG |
| 5. | IMP F Primer | ACATTTCCATAGCGACAGCACG |
| | | GGCGGAAT |
| 6. | IMP R Primer | GGACTTTGGCCAAGCTTCTAAA |
| | | TTTGCGTC |
| 7. | VIM F Primer | CGTGCAGTCTCCACGCACT |
| 8. | VIM R Primer | TCGAATGCGCAGCACCGGGATA |
| | | G |
| 9. | OXA-48-like | GGCTGTGTTTTTGGTGGCATCG |
| | F Primer | ATTATC |
| 10. | OXA-48-like | TCCCACTTAAAGACTTGGTGTT |
| | R Primer | CATCC |
| 11. | DHA F Primer | ACGGGCCGGTAATGCGGATCTG |
| | | GA |
| 12. | DHA R Primer | TATTCGCCAGAATCACAATCGC |
| | | CACCTGT |
| 13. | CMY F Primer | CCGCGGCGAAATTAAGCTCAGC |
| | | GA |
| 14. | CMY R Primer | CCAAACAGACCAATGCTGGAGT |
| | | TAG |
| 15. | EK F Primer | ATTCGTTGCACTATCGTTAACT |
| | | GAATACA |
| 16. | EK R Primer | CTGTACCGTCGGACTTTCCAGA |
| | | C |

TABLE 11
Exemplary Probe Sequences for
Antibiotic Resistance Genes

| SEQ ID | | |
|---|---|---|
| NO | Name | Sequence |
| 17. | NDM 5′ Probe | GCGACCGGCAGGTTGATCT |
| 18. | NDM 3′ Probe | CATGTCGAGATAGGAAGTGTGCTGC |
| 19. | KPC 5′ Probe | CGGAACCATTCGCTAAACTC |
| 20. | KPC 3′ Probe | AGGCGCAACTGTAAGTTACCG |
| 21. | IMP 5′ Probe | GCTTAATTCTCAATCTATCCCCACGTAT |
| 22. | IMP 3′ Probe | CTCCAGATAACGTAGTGGTTTGGCTG |
| 23. | VIM 5′ Probe | CTTTCATGACGACCGCGTCGG |
| 24. | VIM 3′ Probe | CTCTAGAAGGACTCTCATCGAGC |
| 25. | OXA-48-like | ATTTTAAAGGTAGATGCGGG |
| | 5′ Probe | |
| 26. | OXA-48-like | CGCCCTGTGATTTATGTTCA |
| | 3′ Probe | |
| 27. | DHA 5′ Probe | GTTTTArGCACCCAGGAAGC |
| 28. | DHA 3′ Probe | TCTGCTGCGGCCAGTCATA |
| 29. | CMY 5′ Probe | GCGGCTGCCAGTTTTGATAA |
| 30. | CMY 3′ Probe | GTGGCTAAGTGCAGCAGGC |
| 31. | EK 5′ Probe | CGTTCCACTAACACACAAGCTGATTCAG |
| 32. | EK 3′ Probe | ATCTCGGTTGATTTCTTTTCCTCGGG |

TABLE 12
Exemplary Primer Sequences for
Antibiotic Resistance Genes

| SEQ ID | | |
|---|---|---|
| NO | Name | Sequence |
| 33. | CTX-M 14 F | ACGCTTTCCAATGTGCAGTACCAGTA |
| | Primer | |
| 34. | CTX-M 14 R | TGCGATCCAGACGAAACGTCTCATCG |
| | Primer | |
| 35. | CTX-M 15 F | GTGATACCACTTCACCTCGGGCAA |
| | Primer | |
| 36. | CTX-M 15 R | AATACATCGCGACGGCTTTCTGCC |
| | Primer | |
| 37. | ermA F Primer | AGAATTACCTTTGAAAGTCAGGC |
| 38. | ermA R Primer | GCTTCAAAGCCTGTCGGAATTGGTTT |
| 39. | ermB F Primer | GGGCATTTAACGACGAAACTGGCTA |
| 40. | ermB R Primer | GTGTTCGGTGAATATCCAAGGTACGC |
| 41. | mecA F Primer | ATGTTGGTCCCATTAACTCTGAAGAA |
| 42. | mecA R Primer | CACCTGTTTGAGGGTGGATAGCAGTA |
| 43. | mefA F Primer | GCAGGGCAAGCAGTATCATTAATCAC |
| 44. | mefA R Primer | AATTAAATCAGCACCAATCATTATCT |
| | | TCTTCC |
| 45. | SHV F Primer | AAGCTGCTGACCAGCCAGCGTCTGA |
| 46. | SHV R Primer | CGGCGATTTGCTGATTTCGCTCG |
| 47. | TEM F Primer | TGCAGTGCTGCCATAACCATGAGTGA |
| 48. | TEM R Primer | AGCGCAGAAGTGGTCCTGCAACTTT |
| 49. | vanA F Primer | CAGTACGGAATCTTTCGTATTCATCA |
| | | GGA |
| 50. | vanA R Primer | CAGTTCGGGAAGTGCAATACCTGCA |
| 51. | vanB F Primer | AATTGAGCAAGCGATTTCGGGCTGT |
| 52. | vanB R Primer | CGTTTAGAACGATGCCGCCATCCT |
| 53. | Streptococcus | CCTTGGACGGAAATGTAGCTGGCA |
| | pneumoniae | |
| | F Primer | |
| 54. | Streptococcus | AATCACATGGTTGACACCTGCTGTG |
| | pneumoniae | |
| | R Primer | |

TABLE 13
Exemplary Probe Sequences for Antibiotic
Resistance Genes

| SEQ ID | | |
|---|---|---|
| NO | Name | Sequence |
| 57. | CTX-M 14 5′ | CTGTCGAGATCAAGCCTGCCGA |
| | Probe | |
| 58. | CTX-M 14 3′ | ACAAATTGATTGCCCAGCTCGGT |
| | Probe | |
| 59. | CTX-M 15 5′ | AATCAGCGGCGCACGATCTTT |
| | Probe | |
| 60. | CTX-M 15 3′ | AATGCTCGCTGCACCGGTGGTAT |
| | Probe | |
| 61. | ermA 5′ Probe | AAATCTGCAACGAGCTTTGGG |
| 62. | ermA 3′ Probe | GTTTATAAGTGGGTAAACCGTGAA |
| | | TATC |
| 63. | ermB 5′ Probe | TTCGTGTCACTTTAATTCACCAAG |
| | | AT |
| 64. | ermB 3′ Probe | AAAGCCATGCGTCTGACATCT |
| 65. | mecA 5′ Probe | AAGCTCCAACATGAAGATGGCT |
| 66. | mecA 3′ Probe | AGATGGCAAAGATATTCAACTAAC |
| 67. | mefA 5′ Probe | AGTGCCATCTTGCAAATGGCGAT |
| 68. | mefA 3′ Probe | TGCAATTGGTGTGTTAGTGGATCG |
| | | TCATGATA |
| 69. | SHV 5′ Probe | CAGTGGATGGTGGACGATCGGGT |
| 70. | SHV 3′ Probe | TTGTGGTGATTTATCTGCGGGATA |
| | | CT |
| 71. | TEM 5′ Probe | CGCCAGTTAATAGTTTGCGCAACG |
| 72. | TEM 3′ Probe | AAAGCGGTTAGCTCCTTCGGTCCT |
| 73. | vanA 5′ Probe | CGTTCAGTACAATGCGGCCGTTA |
| 74. | vanA 3′ Probe | CTGCGGGAACGGTTATAACTGC |
| 75. | vanB 5′ Probe | GCACCCGATATACTTTCTTTGCC |
| 76. | vanB 3′ Probe | CGCCGACAATCAAATCATCCT |
| 77. | Streptococcus | TTGACCAGTTCCGAGCAAATGGTA |
| | pneumoniae | |
| | 5′ Probe | |
| 78. | Streptococcus | GACAGTATCGATGTTCCAGCAGCT |
| | pneumoniae | |
| | 3′ Probe | |

TABLE 14
Exemplary Primer Sequences for
Antibiotic Resistance Genes

| SEQ ID | | |
|---|---|---|
| NO | Name | Sequence |
| 187 | CTX-M-15 | CCTCGGGCAATGGCGCAAAC |
| | Forward | |
| | Primer #2 | |
| 188 | CTX-M-15 | ATCGCGACGGCTTTCTGCCT |
| | Reverse | TA |
| | Primer #2 | |
| 189 | vanA Forward | TATTCATCAGGAAGTCGAGC |
| | Primer #2 | CGGA |
| 190 | vanB Reverse | AAGATCAACACGGGCAAGCC |
| | Primer #2 | CTCT |
| 191 | mecC Forward | ATGTGGGTCCAATTAATTCT |
| | Primer | GACGAG |
| 192 | mecC Reverse | CTCCAGTTTTTGGTTGTAAT |
| | Primer | GCTGTA |

TABLE 15
Exemplary Probe Sequences for
Antibiotic Resistance Genes

| SEQ ID | | |
|---|---|---|
| NO | Name | Sequence |
| 193 | CTX-M-15 5′ | GGGCGCAGCTGGTGACAT |
| | Probe #2 | |
| 194 | CTX-M-15 3′ | AAGATCGTGCGCCGCTGATT |
| | Probe #2 | |
| 195 | vanA 5′ Probe | GCAGTTATAACCGTTCCCGCAG |
| | #2 | |
| 196 | vanA 3′ Probe | TAACGGCCGCATTGTACTGAACG |
| | #2 | |
| 197 | mecA 5′ Probe | AAAGGCTATAAAGATGATGCAG |
| | #2 | |
| 198 | mecA 3′ probe | GAGTATTTATAACAACATGAAAA |
| | #2 | ATGATT |
| 199 | mecC 5′ Probe | GGCTTAGAACGCCTCTATGAT |
| 200 | mecC 3′ Probe | AGAGTACAAGAAAGTATTTATAA |
| | | ACATATGA |

For example, any of the primers and/or probes shown in Tables 16 and 17 can be used for amplifying a target nucleic acid characteristic of OXA-23-like. For example, in some embodiments, a target nucleic acid characteristic of OXA-23-like may be amplified in the presence of a forward primer that includes the oligonucleotide sequence AGATTGTTCAAGGACATAATCAGGTGA (SEQ ID NO: 183) and a reverse primer that includes the oligonucleotide sequence GGTAAATGACCTTTTCTCGCCCTTC (SEQ ID NO: 184). In some embodiments, an amplicon produced using these primers is detected by hybridization using a 5′ capture probe that includes the oligonucleotide sequence CTCAGGTGTGCTGGTTATTCA (SEQ ID NO: 185) and/or a 3′ capture probe that includes the oligonucleotide sequence GCCCTGATCGGATTGGAGAA (SEQ ID NO: 186) to detect the presence of Staphylococcus spp. in a biological sample. In some embodiments, the 5′ capture probe and/or the 3′ capture probe is conjugated to a magnetic nanoparticle.

TABLE 16
Exemplary Primers for Amplification/
Detection of OXA-23-like

| SEQ ID | | |
|---|---|---|
| NO | Name | Sequence |
| 183 | OXA-23-like | AGATTGTTCAAGGA |
| | Forward Primer | CATAATCAGGTGA |
| 184 | OXA-23-like | GGTAAATGACCTTT |
| | Reverse Primer | TCTCGCCCTTC |

TABLE 17
Exemplary Probes for Detection
of OXA-23-like

| SEQ ID | | |
|---|---|---|
| NO | Name | Sequence |
| 185 | OXA-23-like | CTCAGGTGTGCTGG |
| | 5′ Capture Probe | TTATTCA |
| 186 | OXA-23-like | GCCCTGATCGGATT |
| | 3′ Capture Probe | GGAGAA |

In some embodiments, one or more of the following primers may be used for amplification of a universal CTX-M amplicon: (i) a first degenerate forward primer comprising the nucleotide sequence of CGTTTTCCIATGTGCAGTACCAGTAAGGTTATGGC (SEQ ID NO: 285) and/or a second degenerate forward primer comprising the nucleotide sequence of CGTTTTGCIATGTGCAGTACCAGTAAGGTGATGGC (SEQ ID NO: 286) and/or (ii) a first degenerate reverse primer comprising the nucleotide sequence of GGTGAGGTGGTGTCGCGCGGGTCGCCIGGGAT (SEQ ID NO: 287) and/or a second degenerate reverse primer comprising the nucleotide sequence of GGTGAGGTGGTGTCTCTCGGGTCGCCIGGGAT (SEQ ID NO: 288). In some embodiments, the universal CTX-M target nucleic acid is amplified in the presence of (i) a first degenerate forward primer comprising the nucleotide sequence of CGTTTTCCIATGTGCAGTACCAGTAAGGTTATGGC (SEQ ID NO: 285) and a second degenerate forward primer comprising the nucleotide sequence of CGTTTTGCIATGTGCAGTACCAGTAAGGTGATGGC (SEQ ID NO: 286) and (ii) a first degenerate reverse primer comprising the nucleotide sequence of GGTGAGGTGGTGTCGCGCGGGTCGCCIGGGAT (SEQ ID NO: 287) and a second degenerate reverse primer comprising the nucleotide sequence of GGTGAGGTGGTGTCTCTCGGGTCGCCIGGGAT (SEQ ID NO: 288).

In some embodiments, one or more of the following probes may be used for detection of a universal CTX-M amplicon: (i) one or more (e.g., 1, 2, or all 3) 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) one or more (e.g., 1, 2, or all 3) 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291). In some embodiments, 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291), are used for detection of the universal CTX-M amplicon.

In some embodiments, one or more of the primers and/or probes set forth in Table 26 may be used.

Any suitable internal control primers and/or probes may be used in any of the panels or subpanels described herein. Exemplary internal control primers that can be used are set forth in Table 18. Exemplary internal control probes that can be used for the detection of internal control amplicons are set forth in Table 19.

TABLE 18
OIC Primer Sequences

| SEQ ID | | |
|---|---|---|
| NO | Name | Sequence |
| 55 | OIC F Primer | GGAAATCTAACGAGAGAGCATGCT |
| 56 | OIC R Primer | CGATGCGTGACACCCAGGC |

TABLE 19
OIC Probe Sequences

| SEQ ID | | |
|---|---|---|
| NO | Name | Sequence |
| 79 | OIC 3′ Probe | GAGACGTTTTGGATA |
| | | CATGTGAAAGAAGGC |
| 80 | OIC 5′ Probe | CGATGGTTCACGGGA |
| | | TTCTGCAATTC |

In some embodiments, any of the preceding primers or probes may include one or more modified bases, for example, 2,6-Diaminopurine (abbreviated herein as “/i6diPr/”), deoxyinosine (abbreviated herein as “/ideoxyl/”), nitroindole (abbreviated herein as/35NiTInd/or NitInd) or other modified bases known in the art. In some embodiments, any of the preceding primers or probes may include inosine (abbreviated as “I”).

### Assay Reagents

The methods and compositions (e.g., systems, devices, kits, or cartridges) described herein may include any suitable reagents, for example, magnetic particles, surfactants, buffer components, additives, chelating agents, and the like. The surfactant may be selected from a wide variety of soluble non-ionic surface active agents including surfactants that are generally commercially available under the IGEPAL® trade name from GAF Company. The IGEPAL® liquid non-ionic surfactants are polyethylene glycol p-isooctylphenyl ether compounds and are available in various molecular weight designations, for example, IGEPAL® CA720, IGEPAL® CA630, and IGEPAL® CA890. Other suitable non-ionic surfactants include those available under the trade name TETRONIC® 909 from BASF Corporation. This material is a tetra-functional block copolymer surfactant terminating in primary hydroxyl groups. Suitable non-ionic surfactants are also available under the ALPHONIC® trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names SYNPERONIC® PE series (ICI), PLURONIC® series (BASF), Supronic, MONOLAN®, PLURACARE®, and PLURODAC®, polysorbate surfactants, such as TWEEN® 20 (PEG-20 sorbitan monolaurate), and glycols such as ethylene glycol and propylene glycol.

Such non-ionic surfactants may be selected to provide an appropriate amount of detergency for an assay without having a deleterious effect on assay reactions. In particular, surfactants may be included in a reaction mixture for the purpose of suppressing non-specific interactions among various ingredients of the aggregation assays of the invention. The non-ionic surfactants are typically added to the liquid sample prior in an amount from 0.01% (w/w) to 5% (w/w).

The non-ionic surfactants may be used in combination with one or more proteins (e.g., albumin, fish skin gelatin, lysozyme, or transferrin) also added to the liquid sample prior in an amount from 0.01% (w/w) to 5% (w/w).

Furthermore, the assays, methods, and cartridge units of the invention can include additional suitable buffer components (e.g., Tris base, selected to provide a pH of about 7.8 to 8.2 in the reaction milieu); and chelating agents to scavenge cations (e.g., ethylene diamine tetraacetic acid (EDTA), EDTA disodium, citric acid, tartaric acid, glucuronic acid, saccharic acid or suitable salts thereof).

### Contamination Control

One potential problem in the use of amplification methods such as PCR as an analytical tool is the risk of having new reactions contaminated with old, amplified products. Such contamination could potentially affect downstream sequencing results as well. Potential sources of contamination include a) large numbers of target organisms in clinical specimens that may result in cross-contamination, b) plasmid clones derived from organisms that have been previously analyzed and that may be present in larger numbers in the laboratory environment, and c) repeated amplification of the same target sequence leading to accumulation of amplification products in the laboratory environment. A common source of the accumulation of the PCR amplicon is aerosolization of the product. Typically, if uncontrolled aerosolization occurs, the amplicon will contaminate laboratory reagents, equipment, and ventilation systems. When this happens, all reactions will be positive, and it is not possible to distinguish between amplified products from the contamination or a true, positive sample. In addition to taking precautions to avoid or control this carry-over of old products, preferred embodiments include a blank reference reaction in every PCR experiment to check for carry-over. For example, carry-over contamination will be visible on the agarose gel as faint bands or fluorescent signal when TaqMan® probes, MolBeacons®, or intercalating dyes, among others, are employed as detection mechanisms. Furthermore, it is preferred to include a positive sample. As an example, in some embodiments, contamination control is performed using any of the approaches and methods described in WO 2012/054639. In some embodiments, a bleach solution is used to neutralize potential amplicons, for example, in a reaction tube of a T2Dx® device being used to perform a method of the invention. In some embodiments, contamination control includes the use of ethylene oxide (EtO) treatment, for example, of cartridge components.

Typically, the instrumentation and processing areas for samples that undergo amplification are split into pre- and post-amplification zones. This minimizes the chances of contamination of samples with amplicon prior to amplification. For example, the T2DX® instrument design is such that the pre- and post-amplification instrumentation and processing areas are integrated into a single instrument. This is made possible as described in the sections below.

Amplifying Multiple Amplicons Characteristic of a Target for Improved Sensitivity and/or Specificity

In some embodiments, the methods of the invention may involve amplification, detection, and/or sequencing of more than one amplicon characteristic of a target in a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum. In some embodiments, amplification of more than one target nucleic acid characteristic of a target increases the total amount of amplicons characteristic of the target in an assay (in other words, the amount of analyte is increased in the assay). This increase may allow, for example, an increase in sensitivity and/or specificity of detection of the target compared to a method that involves amplification and detection of a single amplicon characteristic of a target, e.g., for T2MR detection. In some embodiments, the methods of the invention may involve amplifying 2, 3, 4, 5, 6, 7, 8, 9, or 10 amplicons characteristic of a species.

In some embodiments, multiple (e.g., 2, 3, 4, 5, 6, 7, 8, 9, or 10) single-copy loci from a target are amplified and detected. In some embodiments, 2 single-copy loci from a target are amplified and detected. In some embodiments, amplification and detection of multiple single-copy loci from a species may allow for a sensitivity of detection comparable with methods that involve detecting an amplicon that is derived from a multi-copy locus. In some embodiments, methods involving detection of multiple single-copy loci amplified from a microbial species can detect from about 1-10 cells/mL (e.g., 1, 2, 3, 4, 5, 6, 7, 8, 9 or 10 cells/mL) of the microbial species in a liquid sample. In some embodiments, methods involving detection of multiple single-copy loci amplified from a target have at least 95% correct detection when the microbial species is present in the liquid sample at a frequency of less than or equal to 5 cells/mL (e.g., 1, 2, 3, 4, or 5 cells/mL) of liquid sample.

The invention also provides embodiments in which at least three amplicons are produced by amplification of two target nucleic acids, each of which is characteristic of a target. For example, in some embodiments, a first target nucleic acid and a second target nucleic acid to be amplified may be separated (for example, on a chromosome or on a plasmid) by a distance ranging from about 50 base pairs to about 1000 1500 base pairs (bp), e.g., about 50, 100, 150, 200, 250, 300, 400, 500, 600, 700, 800, 900, or 1000, 1100, 1200, 1300, 1400, or 1500 bp base pairs. In some embodiments, a first target nucleic acid and a second target nucleic acid to be amplified may be separated (for example, on a chromosome or on a plasmid) by a distance ranging from about 50 bp to about 1000 bp (e.g., about 50, 100, 150, 200, 250, 300, 400, 500, 600, 700, 800, 900, or 1000 bp). In some embodiments the first target nucleic acid and the second target nucleic acid to be amplified may be separated by a distance ranging from about 50 bp to about 1500 bp, from about 50 bp to about 1400 bp, from about 50 bp to about 1300 bp, from about 50 bp to about 1200 bp, from about 50 bp to about 1100 bp, from about 50 bp to about 1000 bp, from about 50 bp to about 950 bp, from about 50 bp to about 900 bp, from about 50 bp to about 850 bp, from about 50 bp to about 800 bp, from about 50 bp to about 800 bp, from about 50 bp to about 750 bp, from about 50 bp to about 700 bp, from about 50 bp to about 650 bp, from about 50 bp to about 600 bp, from about 50 bp to about 550 bp, from about 50 bp to about 500 bp, from about 50 bp to about 500 bp, from about 50 bp to about 450 bp, from about 50 bp to about 400 bp, from about 50 bp to about 350 bp, from about 50 bp to about 300 bp, from about 50 bp to about 250 bp, from about 50 bp to about 200 bp, from about 50 bp to about 150 bp, or from about 50 bp to about 100 bp. In some embodiments, amplification of the first and second target nucleic acids using individual primer pairs (each having a forward and a reverse primer) may lead to amplification of an amplicon that includes the first target nucleic acid, an amplicon that includes the second target nucleic acid, and an amplicon that contains both the first and the second target nucleic acid. This may result in an increase in sensitivity of detection of the target compared to samples in which the third amplicon is not present. In any of the preceding embodiments, amplification may be by asymmetric PCR.

The invention provides magnetic particles decorated with nucleic acid probes to detect two or more amplicons characteristic of a target. For example, in some embodiments, the magnetic particles include two populations, wherein each population is conjugated to probes such that the magnetic particle that can operably bind each of the two or more amplicons. For instance, in embodiments where two target nucleic acids have been amplified to form a first amplicon and a second amplicon, a pair of particles each of which have a mix of capture probes on their surface may be used. In some embodiments, the first population of magnetic particles may be conjugated to a nucleic acid probe that operably binds a first segment of the first amplicon and a nucleic acid probe that operably binds a first segment of the second amplicon, and the second population of magnetic particles may be conjugated to a nucleic acid probe that operably binds a second segment of the first amplicon and a nucleic acid probe that operably binds a second segment of the second amplicon. For instance, one particle population may be conjugated with a 5′ capture probe specific to the first amplicon and a 5′ capture probe specific to second amplicon, and the other particle population may be conjugated with a 3′ capture probe specific to the first amplicon and a 3′ capture probe specific to the second amplicon.

In such embodiments, the magnetic particles may aggregate in the presence of the first amplicon and aggregate in the presence of the second amplicon. Aggregation may occur to a greater extent when both amplicons are present.

In some embodiments, a magnetic particle may be conjugated to two, three, four, five, six, seven, eight, nine, or ten nucleic acid probes, each of which operably binds a segment of a distinct target nucleic acid. In some embodiments, a magnetic particle may be conjugated to a first nucleic acid probe and a second nucleic acid probe, wherein the first nucleic acid probe operably binds to a first target nucleic acid, and the second nucleic acid probe operably binds to a second target nucleic acid. In other embodiments, a magnetic particle may be conjugated to a first nucleic acid probe that operably binds a first target nucleic acid, a second nucleic acid probe that operably binds a second target nucleic acid, and a third nucleic acid that operably binds a third target nucleic acid. In yet other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, and a fourth nucleic acid probe that operably binds a fourth target nucleic acid. In still other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, a fourth nucleic acid probe that operably binds a fourth target nucleic acid, and a fifth nucleic acid probe that operably binds a fifth target nucleic acid. In some embodiments, one population of magnetic particles includes the 5′ capture probe for each amplicon to be detected, and the other population of magnetic particles includes the 3′ capture probe for each amplicon to be detected.

### Kits

The invention provides kits and articles of manufacture that can be used for carrying out the methods described herein. The kit may include one or more containers for holding the components of the kit (e.g., tubes (e.g., microcentrifuge tubes), plates (e.g., microtiter plates), trays, packaging materials (e.g., boxes), and the like. The kit may also include instructions (e.g., printed instructions for using the kit).

For example, a kit may include one or more, or all, of the following: one or more containers (e.g., tubes) that contain erythrocyte lysis buffers, one or more containers containing buffers or buffered solutions (e.g., TE buffer); one or more containers that contain primers (e.g., any of the primers described herein), one or more containers that contain control nucleic acids or total process controls, one or more containers containing lysis reagents (e.g., beads for beadbeating), and/or one or more containers containing amplification reagents (e.g., buffers, thermostable DNA polymerases, nucleotides, magnesium (e.g., MgCl₂), and the like). The kit may further include reagents for sequencing (e.g., buffers, library preparation reagents, enzymes, adaptors, and the like). The kit may further include reagents for T2MR detection (e.g., magnetic particles, probes, conjugated magnetic particles, and the like).

### EXAMPLES

The following examples are put forth so as to provide those of ordinary skill in the art with a complete disclosure and description of how the devices, systems, and methods described herein are performed, made, and evaluated, and are intended to be purely exemplary of the invention and are not intended to limit the scope of what the inventors regard as their invention.

### Example 1: T2AMR Panels for Detection of ≥99% of Blood-Borne Pathogens and Common Resistance Genes

This example describes direct-from-blood sample, sensitive (≤10 CFU/mL limit of detection, e.g., 1-10 CFU/mL), and rapid detection of 99% of pathogens that are detected by blood culture in hospitals in the U.S. and worldwide. These panels can be used to identify the relevant pathogens and/or resistance markers in a rapid manner to triage patients onto the correct therapy faster than standard of care, as well as to circumvent resistance mechanisms early in treatment. These panels can also provide confidence that antibiotic therapy can be de-escalated upon a negative result. Such de-escalation of empiric therapy within 3 to 5 hours is expected to dramatically reduce the over-prescription of antibiotics and help curtail the spread of resistance.

The multiple-level target strategy of pan, genus, species, and resistance gene results provides redundancy for true infections. For instance, if a patient were infected with MRSA, it is expected that the pan-Gram positive, Staphyloccocus spp., S. aureus and mecA channels will all be positive. Multiple positive results from independent primer sets will provide physicians with confidence that the species-level pathogen is present, and simultaneously provide broad inclusivity of less prevalent species identified with the pan or genus level results.

One exemplary panel includes 36 direct-from-blood results to achieve inclusivity of 99% of blood-borne pathogens and common resistance genes, consisting of 2 pan-level results, 10 genus level results, 5 Gram positive species results, 6 Gram negative species results, and 13 resistance genes (Table 20). Table 21 shows an exemplary split of the results into four groups of 10-plex reactions, including an internal control. However, it is to be understood that the results can be split into different groups.

TABLE 20
Exemplary Panel

| Result Type | N | Results |
|---|---|---|
| Pan-level | 2 | Gram positive bacteria, Gram negative bacteria |
| Genus level | 10 | Acinetobacter spp., Citrobacter spp., Corynebacterium spp., Enterobacter |
| | | spp., Enterobacteriaceae, Enterococcus spp., Mycobacterium spp., |
| | | Staphylococcus spp., Coagulase negative Staphylococcus spp., |
| | | Streptococcus spp. |
| Gram pos. | 5 | E. faecium, S. aureus, S. pneumoniae, S. pyogenes, S. viridans |
| species | | |
| Gram neg. | 6 | A. baumannii, E. coli, Enterobacter cloacae complex, H. influenzae, K. |
| species | | pneumoniae, P. aeruginosa |
| Resistance | 13 | mecA, mefA/E, vanA/B, ermA/B, KPC, NDM, VIM, IMP, OXA, SHV, AmpC |
| genes | | (CMY, DHA), CTX-M 14/15, TEM |

TABLE 21
Exemplary split of n = 36 results into four groups of 10-plex reactions, including an
internal control

| Multiplex Group | | | | |
|---|---|---|---|---|
| ID | A | B | C | D |
| Theme | Gram negative, | Gram positive, | Major antibiotic | Low prevalence |
| | high prevalence | high prevalence | resistance genes | targets |
| Result 1 | Pan Gram positive | Enterococcus spp. | KPC | Acinetobacter |
| | | | | spp. |
| Result 2 | Pan Gram negative | E. faecium | NDM | A. baumannii |
| Result 3 | Enterobacteriaceae | Streptococcus | VIM | Corynebacterium |
| | | spp. | | spp. |
| Result 4 | E. coli | S. pneumoniae | IMP | Mycobacterium |
| | | | | spp. |
| Result 5 | K. pneumoniae | S. pyogenes | OXA | H. influenzae |
| Result 6 | Enterobacter spp. | S. viridans | mecA/C | ermA/B |
| Result 7 | Enterobacter | Staphylococcus | vanA/vanB | SHV |
| | cloacae complex | spp. | | |
| Result 8 | Citrobacter spp. | Coagulase | CTX-M 14, CTX- | TEM |
| | | negative | M 15 | |
| | | Staphylococcus | | |
| | | spp. | | |
| Result 9 | P. aeruginosa | S. aureus | mefA/E | AmpC |
| | | | | (CMY/DHA) |
| Result 10 | Internal control | Internal control | Internal control | Internal control |

Additional T2AMR panels are also provided herein. See, e.g., Tables 22-24.

Panels can be selected to maximize coverage of relevant targets. The panel members can be compared to epidemiological studies for blood-borne infections and/or resistance markers, and the percentage of coverage can be calculated by counting the isolates that would be detected by a panel design and dividing by total isolates in the study. While some studies list all species individually, other studies use genera or non-taxonomic groups without individually identifying species in the group. To simplify calculations, the following groups were defined by their most prevalent members: coagulase negative Staphylococcus (CoNS) includes S. epidermidis, S. haemolyticus, S. lugdunensis, and S. hominis; S. viridans (also referred to as Viridans group Streptococcus) includes S. anginosus, S. mitis, and S. oralis; and Enterobacteriaceae contains the genera Klebsiella, Enterobacter, Citrobacter, Serratia, Proteus, and Morganella. In these calculations, if the published source designated an “other” category with unnamed species, no percentage was awarded to the panel.

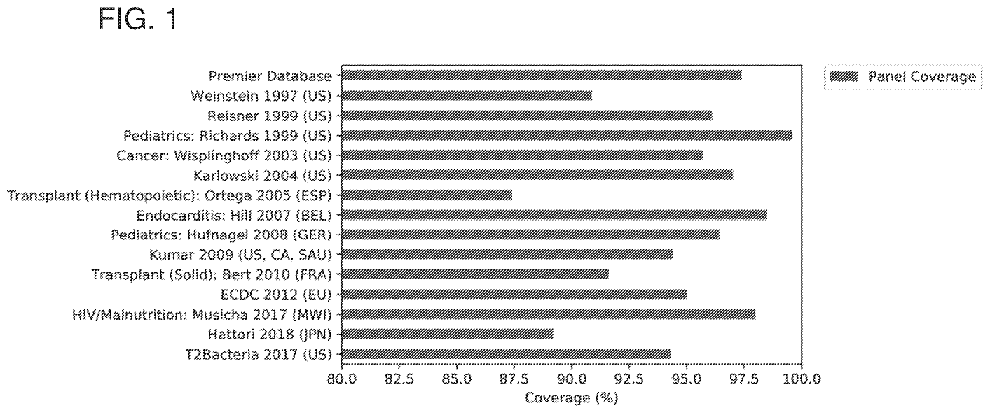

For example, the panel shown in Table 22 covers >90% of species in most epidemiological studies. Addition of Salmonella, Clostridium, and other fungal (Aspergillus/Cryptococcus) species improves coverage of species in most epidemiological studies to >95% (see Table 23). In some examples, Gram negative organisms can be combined with typically Gram negative resistance genes (two 10-plex panels), and Gram positive organisms can be with typically Gram positive resistance genes (two 10-plex panels). To cover >99% of infections, the Pan Gram positive and Pan Gram negative channels can be included in the panels (for example, by omitting the Enterobacteriaceae and/or Mycobacterium channels) (see Table 24 and FIG. 1). In some examples, detection of Neisseria spp. can be substituted with Stenotrophomonas spp. (e.g., Stenotrophomonas maltophilia) due to the unique treatment for these pathogens. Since infection by Acinetobacter spp. other than Acinetobacter baumannii are very rare, this channel may be omitted to allow for additional detection of resistance genes. In another example, the Clostridium channel may be substituted with an anaerobes channel that detects both Clostridium spp. and Bacteroides spp. In some examples, the mefA/E channel may be omitted or substituted with a different target gene (e.g., another resistance target). In another example, the C. dublinensis channel may be omitted to allow for two separate channels that allow for differentiation between C. krusei and C. glabrata. In other examples, a Pan Fungal channel can be included. In these layouts, users can select subpanels for detection individually or in combinations.

TABLE 22
Exemplary T2AMR Panel

| | | Major | | |
|---|---|---|---|---|
| Gram negative, high | Gram positive, high | resistance | Low prevalence | |
| prevalence | prevalence | genes | targets | Fungal |
| Pan Gram positive | Enterococcus spp. | KPC | Acinetobacter | Pan Fungal |
| | | | spp. | |
| Pan Gram negative | E. faecium | NDM | A. baumannii | Candida spp. |
| Enterobacteriaceae | Streptococcus spp. | VIM | Corynebacterlum | C. albicans |
| | | | spp. | |
| E. coli | S. pneumoniae | IMP | Mycobacterium | C. tropicalis |
| | | | spp. | |
| K. pneumoniae | S. pyogenes | OXA | H. influenzae | C. dublinensis |
| Enterobacter spp. | Viridans Group | mecA/C | ermA/B | C. parapsilosis |
| Enterobacter cloacae | Staphylococcus spp. | vanA/B | SHV | C. krusei/C. |
| complex | | | | glabrata |
| Citrobacter spp. | Coagulase negative | CTX-M 14/15 | TEM | C. auris |
| | Staphylococcus spp. | | | |
| | (CoNS) | | | |
| P. aeruginosa | S. aureus | mefA/E | AmpC | FKS |
| | | | (CMY/DHA) | |
| Internal control (IC) | IC | IC | IC | PDR1 & |
| | | | | ERG11 |

TABLE 23
Exemplary T2AMR Panel

| Gram negative 1 | Gram negative 2 | Gram positive 1 | Gram positive 2 | Fungal |
|---|---|---|---|---|
| Enterobacteriaceae | Acinetobacter | Enterococcus | Staphylococcus spp. | Candida spp. |
| | spp. | spp. | | |
| E. coli | A. baumannii | E. faecalis | S. aureus | C. albicans |
| K. pneumoniae | Pseudomonas | E. faecium | CoNS | C. tropicalis |
| | aeruginosa | | | |
| Enterobacter spp. | Neisseria spp. | Streptococcus | Clostridium spp. | C. parapsilosis |
| | | spp. | | |
| Enterobacter cloacae | H. influenzae | S. pneumoniae | Corynebacterium | C. krusei/C. |
| complex | | | spp. | glabrata |
| Citrobacter spp. | KPC | S. pyogenes | mecA/C | C. dublinensis |
| Serratia marcescens | NDM/VIM/IMP | S. agalactiae | vanA/B | C. auris |
| Proteus mirabilis | OXA | Viridans Group | ermA/B | Aspergillus |
| | | | | spp. |
| Salmonella spp. | CTX-M | Mycobacterium | mefA/E | Cryptococcus |
| | | spp. | | spp. |
| IC | IC | IC | IC | IC |

TABLE 24
Exemplary T2AMR Panel

| Gram negative 1 | Gram negative 2 | Gram positive 1 | Gram positive 2 | Fungal |
|---|---|---|---|---|
| Pan Gram negative | Acinetobacter spp. | Pan Gram | Streptococcus spp. | Candida spp. |
| | | positive | | |
| E. coli | A. baumannii | Enterococcus | S. pneumoniae | C. albicans |
| | | spp. | | |
| K. pneumoniae | Pseudomonas | E. faecium | S. pyogenes | C. tropicalis |
| | aeruginosa | | | |
| Enterobacter spp. | Stenotrophomonas | E. faecalis | S. agalactiae | C. parapsilosis |
| | maltophilia | | | |
| Enterobacter cloacae | H. influenzae | Staphylococcus | Viridans Group | C. krusei |
| complex | | spp. | | |
| Citrobacter spp. | KPC | S. aureus | Anaerobes | C. glabrata |
| Serratia marcescens | NDM/VIM/IMP | CoNS | Corynebacterium | C. auris |
| | | | spp. | |
| Proteus mirabilis | OXA | mecA/C | ermA/B | Aspergillus |
| | | | | spp. |
| Salmonella spp. | CTX-M | vanA/B | mefA/E | Cryptococcus |
| | | | | spp. |
| IC | IC | IC | IC | IC |

Any of the panels may include the MCR gene (colistin), which is a Gram negative resistance gene. Any of the panels may include a Klebsiella spp. channel. Any of the panels may identify K. oxytoca and/or K. variicola (e.g., in a single channel).

With respect to the panel shown in Table 24, the panel coverage for the indicated studies is shown below in Table 25.

TABLE 25
Estimated Coverage of Exemplary T2AMR Panel shown in Table 24

| Study | Panel Coverage |
|---|---|
| Premier Database | 97.4 |
| Weinstein et al. Clin. Infect. Dis. 24:584-602, 1997 | 90.9 |
| (US) | |
| Reisner et al. J. Clin. Microbiol. 37(6):2024-2026, | 96.1 |
| 1999 (US) | |
| Pediatrics: Richards et al. Pediatrics 103(4), 1999 | 99.6 |
| 1999 (US) | |
| Cancer: Wisplinghoff et al. Clin. Infect. Dis. | 95.7 |
| 2003(36):1103-1110, 2003 (US) | |
| Karlowski et al. Annals of Clinical Microbiology and | 97 |
| Antimicrobials 3:7, 2004 (US) | |
| Transplant (Hematopoietic): Ortega et al. Ann. | 87.4 |
| Hematol. 84:40-47, 2005 (ESP) | |
| Endocarditis: Hill et al. Eur. Heart J. 28:196-203, | 98.5 |
| 2007 (BEL) | |
| Pediatrics: Hufnagel et al. Eur. J. Pediatr. | 96.4 |
| 167:1149-1159, 2008 (GER) | |
| Kumar et al. Chest 136:1237-1248, 2009 (US, CA, | 94.4 |
| SAU) | |
| Transplant (Solid): Bert et al. Liver Transplant. | 91.6 |
| 16:393-401, 2010 (FRA) | |
| European Center for Disease Prevention and | 95 |
| Control (ECDC) Surveillance Report, “Incidence | |
| and attributable mortality of healthcare-associated | |
| infections in intensive care units in Europe, 2008- | |
| 2012”, 2012 (EU) | |
| HIV/Malnutrition: Musicha et al. Lancet Infect. Dis. | 98 |
| 17:1042-1052, 2017 (MWI) | |
| Hattori et al. Am. J. Infect. Control. 46(12):e75-79, | 89.2 |
| 2018 (JPN) | |
| T2Bacteria 2017 (US) | 94.3 |
| Average | 94.6 |

These results show that the panels described in this Example can detect a very high percentage (e.g., ≥90%, ≥95%, ≥99%, or higher) of blood-borne infections and resistance markers described in epidemiological studies in the U.S. and worldwide. This nearly complete coverage allows for direct-from-blood sample, sensitive (≤10 CFU/mL limit of detection, e.g., 1-10 CFU/mL), and rapid detection of the relevant pathogen(s) and/or resistance markers. The addition of Pan-Gram negative or Pan-Gram positive channels can be used to further increase the percent coverage. These panels can be used to identify the relevant pathogens and/or resistance markers in a rapid manner to triage patients onto the correct therapy faster than standard of care, as well as to circumvent resistance mechanisms early in treatment.

### Example 2: Results from Exemplary T2AMR Panel

Summary

The T2AMR Panel described in this Example includes 40 detection channels that cover species, genus, higher level groupings, and resistance genes implicated in bacterial blood stream infections. The genetic targets were selected based on analysis of specificity with predetermined inclusivity and exclusivity lists, and primers and probes were designed for each target. Primer performance was assayed using a real time PCR analysis of genomic DNA (gDNA) in buffer detected with non-specific fluorescence. Detection with probes was determined using gDNA amplified in blood lysate and a high throughput T2MR reader system. We demonstrated amplification with primers and T2 magnetic resonance (T2MR) detection of all targets with ≤100 copies of gDNA per reaction.

The T2AMR Panel was designed to detect 40 targets implicated in bacterial sepsis. This panel broadly covers major sepsis causing organisms and antibiotic resistance genes that result in multidrug resistant infections. Targets were determined through rigorous bioinformatic analysis of genetic regions and comparison to inclusive and exclusive species and genes. Primers and probes were designed using thermodynamic simulations to determine melting temperature and free energy of duplexes and secondary structures. All experiments were performed with singleplex reaction buffers and either DNA purified from the organism of interest or synthetic DNA.

Initial primer designs were shown to amplify all 40 AMR targets using a real-time PCR method with non-specific DNA detection. T2MR detection was achieved for all 40 AMR targets with gDNA spiked at s 100 copies/reaction in lysate.

Methods

Symmetric singleplex reaction buffers were prepared with primers, and real-time PCR was performed using 10 to 10,000 copies of gDNA spiked in buffers. SYBR® Green was used to non-specifically stain for DNA. Primer efficiency was calculated from results and sensitivity was defined as lowest concentration with 75% positive hit rate. Human whole blood was lysed with detergents. Lysed blood was centrifuged and the pellet was washed with Tris-EDTA. The remaining pellet was suspended in Tris-EDTA and disrupted by bead beating; this suspension is referred to as blood lysate in this Example.

PCR reactions were prepared using asymmetric singleplex reaction buffers with or without blood lysate spiked with ≤100 copies gDNA/reaction. Reactions were amplified and detected using probe conjugated particles and a T2MR reader. Results with a T2MR signal >65 ms were considered positive.

Table 26 shows exemplary primer and probe sequences that have been tested for use in T2AMR panels. A “Y” in the right-hand column indicates that the primer or probe sequence was used in the experiments described in this Example (see Table 27).

TABLE 26
Exemplary primer and probe sequences tested for use in T2AMR panels

| | | | SEQ ID | Used in |
|---|---|---|---|---|
| Target | Type | Sequence | NO: | Example 2? |
| Acinetobacter | F primer | CACTCGTACCGTCTCTGAGGGTGTT | 201. | |
| spp. | | GCA | | |
| Acinetobacter | R primer | CGTAGATTCACGACCKGGACCAGGG | 202. | |
| spp. | | CCT | | |
| Acinetobacter | 3′ probe | ACAATGGTGTTATTAAAAGAGGCGT | 203. | |
| spp. | | GAATGTG | | |
| Acinetobacter | 5′ probe | AACCGTAGTCTAAAGCGGCTTTACC | 204. | |
| spp. | | AGCAACT | | |
| Acinetobacter | F primer | T2Bacteria (see Table 3) | 149. | Y |
| baumannii | | | | |
| Acinetobacter | R primer | T2Bacteria (see Table 3) | 82. | Y |
| baumannii | | | | |
| Acinetobacter | 3′ probe | T2Bacteria (see Table 4) | 95. | Y |
| baumannii | | | | |
| Acinetobacter | 5′ probe | T2Bacteria (see Table 4) | 96. | Y |
| baumannii | | | | |
| Bacteroides | F primer | TATCCGATTATTATGCCAGCCATGT | 205. | Y (as |
| fragilis | | ATTCGA | | Bacteroides |
| | | | | spp.) |
| Bacteroides | R primer | CCAGTGGGTGTAGTGTGTCACGTTG | 206. | Y (as |
| fragilis | | AGC | | Bacteroides |
| | | | | spp.) |
| Bacteroides | 3′ probe | ACTGCTTTGTATGCTTCTTTGGGAA | 207. | Y (as |
| fragilis | | GGTATTC | | Bacteroides |
| | | | | spp.) |
| Bacteroides | 5′ probe | CAGCTTTTCATGCCGTTGGCTATCA | 208. | Y (as |
| fragilis | | G | | Bacteroides |
| | | | | spp.) |
| Citrobacter spp. | F primer | CGCTGGCGCATGACGAGAAG | 209. | Y |
| Citrobacter spp. | R primer | GCTTGCGGAGATGCCGTCATAGGTA | 210. | Y |
| | | CCGGCTT | | |
| Citrobacter spp. | 3′ probe | TAGATGAAGAACCATTTCCAGTCCA | 211. | Y |
| | | TGGAAAC | | |
| Citrobacter spp. | 5′ probe | CGTTGGCGATCAGATGCAG | 212. | Y |
| Clostridium spp. | F primer | GTTTGTAGTACATGAAGCACCTGAG | 213. | Y |
| | | ATAACATC | | |
| Clostridium spp. | R primer | CGGAAAAGAAATCAAAGTAACTGCT | 214. | Y |
| | | AAAAGC | | |
| Clostridium spp. | 3′ probe | ACCACGATATATTAGATGGAAGCGA | 215. | Y |
| | | A | | |
| Clostridium spp. | 5′ probe | GGGAGAATTAGGAGTAGACGTAGTA | 216. | Y |
| | | TT | | |
| Enterobacter | F primer | TGATCCCGTTCTGGATGGTGA | 217. | Y |
| cloacae complex | | | | |
| Enterobacter | R primer | TTCCACATAATCCAGATGGAACCGA | 218. | Y |
| cloacae complex | | CT | | |
| Enterobacter | 3′ probe | GAATCTGAATCACCGCGG | 219. | Y |
| cloacae complex | | | | |
| Enterobacter | 5′ probe | CCAGGATAGCGATGATAATCAC | 220. | Y |
| cloacae complex | | | | |
| Enterobacterales | F primer | TGAGGGAAAGGCGAAAAGAACCTTG | 221. | Y |
| | | GC | | |
| Enterobacterales | R primer | ATGGGTAGATCACCGGGTTTCGGGT | 222. | Y |
| | | CTATA | | |
| Enterobacterales | 3′ probe | TTGTACGTACACGGTTTCAGGTTCT | 223. | Y |
| | | T | | |
| Enterobacterales | 5′ probe | CGCCCGGTTAAGACTCGGTTTCC | 224. | Y |
| Enterococcus | F primer | T2Bacteria (see Table 3) | 83. | Y |
| spp. | | | | |
| Enterococcus | R primer | T2Bacteria (see Table 3) | 84. | Y |
| spp. | | | | |
| Enterococcus | 3′ probe | TGTGGTGGCGATGGCGAGAAGGATA | 225. | Y |
| spp. | | CA | | |
| Enterococcus | 5′ probe | GAAGCCCACCTCAAGATGAGATTTC | 226. | Y |
| spp. | | C | | |
| Enterococcus | F primer | T2Bacteria (see Table 3) | 83. | Y |
| faecalis | | | | |
| Enterococcus | R primer | T2Bacteria (see Table 3) | 84. | Y |
| faecalis | | | | |
| Enterococcus | 3′ probe | AATGAAGATTCAACTCAATAAGAAA | 104. | Y |
| faecalis | | CAACA | | |
| Enterococcus | 5′ probe | TGGATAAGTAAAAGCAACTTGGTT | 103. | Y |
| faecalis | | | | |
| Enterococcus | F primer | T2Bacteria (see Table 3) | 83. | Y |
| faecium | | | | |
| Enterococcus | R primer | T2Bacteria (see Table 3) | 84. | Y |
| faecium | | | | |
| Enterococcus | 3′ probe | T2Bacteria (see Table 4) | 151. | Y |
| faecium | | | | |
| Enterococcus | 5′ probe | T2Bacteria (see Table 4) | 150. | Y |
| faecium | | | | |
| Escherichia coli | F primer | T2Bacteria (see Table 3) | 132. | Y |
| Escherichia coli | R primer | T2Bacteria (see Table 3) | 133. | Y |
| Escherichia coli | 3′ probe | T2Bacteria (see Table 4) | 135 | Y |
| Escherichia coli | 5′ probe | T2Bacteria (see Table 3) | 134. | Y |
| Haemophilus | F primer | CTTTATCAACGTAACGGCATTGGTC | 227. | Y |
| influenzae | | AATAC | | |
| Haemophilus | R primer | CGCATAGGAGGGAAATGGTTATTAC | 228. | Y |
| influenzae | | TTAAAC | | |
| Haemophilus | 3′ probe | GGAAAACATCTGGCTTGTGTGTTTT | 229. | Y |
| influenzae | | TAACC | | |
| Haemophilus | 5′ probe | GGTTGTCATCATTGATGTTCCCGC | 230. | Y |
| influenzae | | | | |
| Klebsiella | F primer | GCCGTCAGTCAGCAGATGCAG | 231. | Y |
| aerogenes | | | | |
| Klebsiella | R primer | GATCCCCTGCTCGGTCAGTAAT | 232. | Y |
| aerogenes | | | | |
| Klebsiella | 3′ probe | CGGCGGACTGGGTACGACATA | 233. | Y |
| aerogenes | | | | |
| Klebsiella | 5′ probe | GCGAAAAGCTCTTTGCCTACCAG | 234. | Y |
| aerogenes | | | | |
| Klebsiella | F primer | TGATCCCGGTGTTGGCGATCGTG | 235. | |
| oxytoca | | | | |
| Klebsiella | R primer | TTCATGATTCCGGTGACGATCAGCG | 236. | |
| oxytoca | | CC | | |
| Klebsiella | 3′ probe | TGCGCTTCGATAAACAGGTTCAGCG | 237. | |
| oxytoca | | G | | |
| Klebsiella | 5′ probe | CCACAGCACCTGGCACACCAG | 238. | |
| oxytoca | | | | |
| Klebsiella | F primer | T2Bacteria (see Table 3) | 85. | Y |
| pneumoniae | | | | |
| Klebsiella | R primer | T2Bacteria (see Table 3) | 86. | Y |
| pneumoniae | | | | |
| Klebsiella | 3′ probe | T2Bacteria (see Table 4) | 108. | Y |
| pneumoniae | | | | |
| Klebsiella | 5′ probe | T2Bacteria (see Table 4) | 107. | Y |
| pneumoniae | | | | |
| Morganella | F primer | CTGCATCCGCGTGCCAATATCG | 239. | |
| morganii | | | | |
| Morganella | R primer | AGCATTTCCGGTTTGAAGAAATGCC | 240. | |
| morganii | | C | | |
| Morganella | 3′ probe | CGGCATCATTCAGCGGGT | 241. | |
| morganii | | | | |
| Morganella | 5′ probe | GGGCTTTCAGGCGGCTTTC | 242. | |
| morganii | | | | |
| Mycobacterium | F primer | TGGTCAGGTGGTCAGCGAAGAGGTC | 243. | Y |
| spp. | | GG | | |
| Mycobacterium | R primer | CGTAGTCGGAGTCGCTGTTCTC | 244. | Y |
| spp. | | | | |
| Mycobacterium | 3′ probe | GGTGACGACGACCTTGCGGGCTTT | 245. | Y |
| spp. | | | | |
| Mycobacterium | 5′ probe | TCTGGGCCACGCGGCCGG | 246. | Y |
| spp. | | | | |
| PanGram primers | F primer | CTCCTACGGGAGGCAGCAGT | 173. | Y |
| PanGram primers | R primer | GTATTACCGCGGCTGCTGGCA | 174. | Y |
| PanGram | 3′ probe | CCGCAGAAGAAGCACCGGCTAACTC | 247. | Y |
| negative | | CGT | | |
| PanGram | 5′ probe | CTGATCCAGCCATGCCGCGTGTATG | 177. | Y |
| negative | | A | | |
| PanGram | 3′ probe | CTAACCAGAAAGCCACGGCTAACTA | 248. | Y |
| positive | | CGT | | |
| PanGram | 5′ probe | TCTGACGGAGCAACGCCGCGTGAGT | 249. | Y |
| positive | | GA | | |
| Proteus spp. | F primer | CGCAGAGGTTTATATGTCAATTCCA | 250. | Y |
| | | GCTA | | |
| Proteus spp. | R primer | TCTTGATAAGCGGCGACCAGCTTAT | 251. | Y |
| | | T | | |
| Proteus spp. | 3′ probe | CAATATTGGATGCTTGTAAAGTTTG | 252. | Y |
| | | AATT | | |
| Proteus spp. | 5′ probe | TTCAACGCCGGGCGTACCTAACGT | 253. | Y |
| Pseudomonas | F primer | T2Bacteria (see Table 3) | 87. | Y |
| aeruginosa | | | | |
| Pseudomonas | R primer | T2Bacteria (see Table 3) | 88. | Y |
| aeruginosa | | | | |
| Pseudomonas | 3′ probe | T2Bacteria (see Table 4) | 154. | Y |
| aeruginosa | | | | |
| Pseudomonas | 5′ probe | T2Bacteria (see Table 4) | 153. | Y |
| aeruginosa | | | | |
| Salmonella spp. | F primer | CGCCGTARCCGTTGCTGATG | 254. | |
| Salmonella spp. | R primer | CGATCACCACGTCTAAACGTACTGC | 255. | |
| | | CGA | | |
| Salmonella spp. | 3′ probe | CAGTACCAGTTCGACTTTGGTCTGC | 256. | |
| Salmonella spp. | 5′ probe | ACTGGTGGCCTGAAATACGATGCGA | 257. | |
| | | ACAAC | | |
| Serratia spp. | F primer | CCTGAAGAAGTCCGGTGCCTTCGTA | 258. | Y |
| Serratia spp. | R primer | AGCCATAAAGTCCATGATGACGACA | 259. | Y |
| | | AC | | |
| Serratia spp. | 3′ probe | CCAAAGTAGAATGGTACTTTCATTG | 260. | Y |
| | | CA | | |
| Serratia spp. | 5′ probe | TCGCGCCCACCAGGGTTAAAC | 261. | Y |
| Staphylococcus | F primer | GCTACTGGTCGTGTTGAACGTGGTC | 262. | Y |
| universal | | AAATCA | | |
| primers | | | | |
| Staphylococcus | R primer | ACAACGCCTGAWCCTACAGTACGTC | 263. | Y |
| universal | | CA | | |
| primers | | | | |
| Staphylococcus | 3′ probe | AGATGTGTCATGTAAACCGAT | 264. | Y |
| aureus | | | | |
| Staphylococcus | 5′ probe | ATACTTCTGCTTTGAATTCAGTATG | 265. | Y |
| aureus | | | | |
| Staphylococcus | 3′ probe | GTTGTGGGTACCGATGATTTCAACT | 266. | Y |
| coagulase | | TC | | |
| negative 1 | | | | |
| Staphylococcus | 5′ probe | AGGTAAAAAGTATACTTCAGCTTTG | 267. | Y |
| coagulase | | AATTTAGTGTG | | |
| negative 1 | | | | |
| Staphylococcus | 3′ probe | TGCTTGATTCTTCTTGCATACCGAT | 268. | Y |
| coagulase | | G | | |
| negative 2 | | | | |
| Staphylococcus | 5′ probe | AAAACTTGACCACGTTGGACAT | 269. | Y |
| coagulase | | | | |
| negative 2 | | | | |
| Stenotrophomon | F primer | CTGATCATGCTGTCGCTGTGGATC | 270. | Y |
| as maltophilia | | | | |
| Stenotrophomon | R primer | GGTGATGGTCAGCACCGGGAA | 271. | Y |
| as maltophilia | | | | |
| Stenotrophomon | 3′ probe | CTGTATTCGATGCCCGACAGTGGTG | 272. | Y |
| as maltophilia | | GGAA | | |
| Stenotrophomon | 5′ probe | CGGCATCTGCATGAGCTTCATGCT | 273. | Y |
| as maltophilia | | | | |
| Streptococcus | F degenerate | GGCATTGGTCCGTCTGTTGATGCAA | 274. | |
| spp. | 1 primer | CTACAAGG | | |
| Streptococcus | F degenerate | GGCATTGGACCGTCAGTTGAAGCAA | 275. | |
| spp. | 2 primer | CTACAAGG | | |
| Streptococcus | R primer | TGGCAAAAGAAAAATACGATCGTAG | 276. | |
| spp. | | TAAACC | | |
| Streptococcus | 3′ probe | TCACTGGTGCTGCTCAAATGG | 277. | |
| spp. | | | | |
| Streptococcus | 5′ probe | ACGTTAACATTGGTACAATTGGACA | 278. | |
| spp. | | CG | | |
| Streptococcus | F primer | CCGTCCGGGTGATCCAGGTGATCAT | 279. | Y |
| group A/B primer | | GGTAAAGG | | |
| primers | | | | |
| Streptococcus | R primer | GTTGGCAGTAATGCTGCCGCGATCT | 280. | Y |
| group A/B primer | | GGCAT | | |
| primers | | | | |
| Streptococcus | 3′ probe | TGGTCAATGGCATATTGAGCAAC | 281. | Y |
| group A | | | | |
| (pyogenes) | | | | |
| Streptococcus | 5′ probe | CTTGCAAATGGCGCATAGAATCG | 282. | Y |
| group A | | | | |
| (pyogenes) | | | | |
| Streptococcus | 3′ probe | CATATTTTGTGTAGAGTACTGTGCA | 283. | Y |
| group B | | ACTT | | |
| (agalactiae) | | | | |
| Streptococcus | 5′ probe | ATTCCAAGTATTAGCAGGTCCATAA | 284. | Y |
| group B | | | | |
| (agalactiae) | | | | |
| Streptococcus | F primer | T2Resistance/T2GNR/T2ARx (see | 53. | Y |
| pneumoniae | | Table 12) | | |
| Streptococcus | R primer | T2Resistance/T2GNR/T2ARx (see | 54. | Y |
| pneumoniae | | Table 12) | | |
| Streptococcus | 3′ probe | T2Resistance/T2GNR/T2ARx (see | 78. | Y |
| pneumoniae | | Table 13) | | |
| Streptococcus | 5′ probe | T2Resistance/T2GNR/T2ARx (see | 77. | Y |
| pneumoniae | | Table 13 | | |
| CMY | F primer | T2Resistance/T2GNR/T2ARx (see | 13. | Y (as |
| | | Table 10) | | AmpC) |
| CMY | R primer | T2Resistance/T2GNR/T2ARx (see | 14. | Y (as |
| | | Table 10) | | AmpC) |
| CMY | 3′ probe | T2Resistance/T2GNR/T2ARx (see | 32. | Y (as |
| | | Table 11) | | AmpC) |
| CMY | 5′ probe | T2Resistance/T2GNR/T2ARx (see | 31. | Y (as |
| | | Table 11) | | AmpC) |
| CTX-M universal | F degenerate | CGTTTTCCTATGTGCAGTACCAGTA | 285. | Y |
| | 1 primer | AGGTTATGGC | | |
| CTX-M universal | F degenerate | CGTTTTGCTATGTGCAGTACCAGTA | 286. | Y |
| | 2 primer | AGGTGATGGC | | |
| CTX-M universal | R degenerate | GGTGAGGTGGTGTCGCGCGGGTCGC | 287. | Y |
| | 1 primer | CTGGGAT | | |
| CTX-M universal | R degenerate | GGTGAGGTGGTGTCTCTCGGGTCGC | 288. | Y |
| | 2 primer | CTGGGAT | | |
| CTX-M universal | 3′ degenerate | GCTTTCACTTTTCTFCAGCACCGCG | 289. | Y |
| | 1 probe | GCC | | |
| CTX-M universal | 3′ degenerate | CGTTTCACTCTGCTTAAGCACCGCC | 290. | Y |
| | 2 probe | GCG | | |
| CTX-M universal | 3′ degenerate | CGTTTCACTTTGCTTGAGCACCGCC | 291. | Y |
| | 3 probe | GT | | |
| CTX-M universal | 5′ degenerate | GGCGGTGTTTAACGTCGGCTCGGTA | 292. | Y |
| | 1 probe | CG | | |
| CTX-M universal | 5′ degenerate | GGCGGTATTCAGCGTAGGTTCAGTG | 293. | Y |
| | 2 probe | CG | | |
| CTX-M universal | 5′ degenerate | CGCGGTGTTGAGCGTCGGCTCAGTA | 294. | Y |
| | 3 probe | CG | | |
| DHA | F primer | T2Resistance/T2GNR/T2ARx (see | 11. | |
| | | Table 10) | | |
| DHA | R primer | T2Resistance/T2GNR/T2ARx (see | 12. | |
| | | Table 10) | | |
| DHA | 3′ probe | T2Resistance/T2GNR/T2ARx (see | 28. | |
| | | Table 11) | | |
| DHA | 5′ probe | T2Resistance/T2GNR/T2ARx (see | 27. | |
| | | Table 11) | | |
| IMP | F primer | T2Resistance/T2GNR/T2ARx (see | 5. | Y |
| | | Table 10) | | |
| IMP | R primer | T2Resistance/T2GNR/T2ARx (see | 6. | Y |
| | | Table 10) | | |
| IMP | 3′ probe | T2Resistance/T2GNR/T2ARx (see | 22. | Y |
| | | Table 11) | | |
| IMP | 5′ probe | T2Resistance/T2GNR/T2ARx (see | 21. | Y |
| | | Table 11) | | |
| KPC | F primer | T2Resistance/T2GNR/T2ARx (see | 3. | Y |
| | | Table 10) | | |
| KPC | R primer | T2Resistance/T2GNR/T2ARx (see | 4. | Y |
| | | Table 10) | | |
| KPC | 3′ probe | T2Resistance/T2GNR/T2ARx (see | 20. | Y |
| | | Table 11) | | |
| KPC | 5′ probe | T2Resistance/T2GNR/T2ARx (see | 19. | Y |
| | | Table 11) | | |
| mcr-1 | F primer | AAATGCCCTACAGACCGACCAAG | 295. | Y |
| mcr-1 | R primer | TGAACGCCACCACAGGCAGTAAA | 296. | Y |
| mcr-1 | 3′ probe | CCAATGATACGCATGATAAACGCT | 297. | Y |
| mcr-1 | 5′ probe | CAAGACTTGCCACGATCAAGC | 298. | Y |
| mecA | F primer | T2Resistance/T2GNR/T2ARx (see | 41. | Y |
| | | Table 12) | | |
| mecA | R primer | T2Resistance/T2GNR/T2ARx (see | 42. | Y |
| | | Table 12) | | |
| mecA | 3′ probe | T2Resistance/T2GNR/T2ARx (see | 66. | Y |
| | | Table 13) | | |
| mecA | 5′ probe | T2Resistance/T2GNR/T2ARx (see | 65. | Y |
| | | Table 13) | | |
| mecC | F primer | T2Resistance/T2GNR/T2ARx (see | 191. | |
| | | Table 14) | | |
| mecC | R primer | T2Resistance/T2GNR/T2ARx (see | 192. | |
| | | Table 14) | | |
| mecC | 3′ probe | T2Resistance/T2GNR/T2ARx (see | 200. | |
| | | Table 15) | | |
| mecC | 5′ probe | T2Resistance/T2GNR/T2ARx (see | 199. | |
| | | Table 15) | | |
| NDM | F primer | T2Resistance/T2GNR/T2ARx (see | 1. | Y |
| | | Table 10) | | |
| NDM | R primer | T2Resistance/T2GNR/T2ARx (see | 2. | Y |
| | | Table 10) | | |
| NDM | 3′ probe | T2Resistance/T2GNR/T2ARx (see | 18. | Y |
| | | Table 11) | | |
| NDM | 5′ probe | T2Resistance/T2GNR/T2ARx (see | 17. | Y |
| | | Table 11) | | |
| OXA-23 | F primer | AGATTGTTCAAGGACATAATCAGGT | 183. | Y |
| | | GA | | |
| OXA-23 | R primer | GGTAAATGACCTTTTCTCGCCCTTC | 184. | Y |
| OXA-23 | 3′ probe | GCCCTGATCGGATTGGAGAA | 186. | Y |
| OXA-23 | 5′ probe | CTCAGGTGTGCTGGTTATTCA | 185. | Y |
| OXA-48 | F primer | T2Resistance/T2GNR/T2ARx (see | 9. | Y |
| | | Table 10) | | |
| OXA-48 | R primer | T2Resistance/T2GNR/T2ARx (see | 10. | Y |
| | | Table 10) | | |
| OXA-48 | 3′ probe | T2Resistance/T2GNR/T2ARx (see | 26. | Y |
| | | Table 11) | | |
| OXA-48 | 5′ probe | T2Resistance/T2GNR/T2ARx (see | 25. | Y |
| | | Table 11) | | |
| vanA | F primer | T2Resistance/T2GNR/T2ARx (see | 49. | Y |
| | | Table 12) | | |
| vanA | R primer | T2Resistance/T2GNR/T2ARx (see | 50. | Y |
| | | Table 12) | | |
| vanA | 3′ probe | T2Resistance/T2GNR/T2ARx (see | 74. | Y |
| | | Table 13) | | |
| vanA | 5′ probe | T2Resistance/T2GNR/T2ARx (see | 73. | Y |
| | | Table 13) | | |
| vanB | F primer | T2Resistance/T2GNR/T2ARx (see | 51. | |
| | | Table 12) | | |
| vanB | R primer | T2Resistance/T2GNR/T2ARx (see | 52. | |
| | | Table 12) | | |
| vanB | 3′ probe | T2Resistance/T2GNR/T2ARx (see | 76. | |
| | | Table 13) | | |
| vanB | 5′ probe | T2Resistance/T2GNR/T2ARx (see | 75. | |
| | | Table 13) | | |
| VIM | F primer | T2Resistance/T2GNR/T2ARx (see | 7. | Y |
| | | Table 10) | | |
| VIM | R primer | T2Resistance/T2GNR/T2ARx (see | 8. | Y |
| | | Table 10) | | |
| VIM | 3′ probe | T2Resistance/T2GNR/T2ARx (see | 24. | Y |
| | | Table 11) | | |
| VIM | 5′ probe | T2Resistance/T2GNR/T2ARx (see | 23. | Y |
| | | Table 11) | | |
| mefA/E | F primer | T2Resistance/T2GNR/T2ARx (see | 43. | Y |
| | | Table 12) | | |
| mefA/E | R primer | T2Resistance/T2GNR/T2ARx (see | 44. | Y |
| | | Table 12) | | |
| mefA/E | 3′ probe | T2Resistance/T2GNR/T2ARx (see | 68. | Y |
| | | Table 13) | | |
| mefA/E | 5′ probe | T2Resistance/T2GNR/T2ARx (see | 67. | Y |
| | | Table 13) | | |
| oxa-24/40 | F primer | GGTGAGGCAATGGCATTGTCAG | 299. | Y |
| oxa-24/40 | R primer | GTAATCGGTTATGTGCAAGGTCATC | 300. | Y |
| | | GG | | |
| oxa-24/40 | 3′ probe | CTTGCAAGCTCTTGATATACTGG | 301. | Y |
| oxa-24/40 | 5′ probe | ACTTCTTGTACTGGTGTAATTTTAA | 302. | Y |
| | | GGG | | |
| strA | F primer | ATCCCTGCATGCCGAACTTCATG | 303. | Y |
| strA | R primer | GCTCTGCTTCATCTGGCGCTG | 304. | Y |
| strA | 3′ probe | AGACCCGTGCATTGAAGAGTT | 305. | Y |
| strA | 5′ probe | CCAGTTCTCTTCGGCGTTAGC | 306. | Y |
| strB | F primer | GCGGCGATTATAGCCGGTCAAATGA | 307. | |
| | | TGA | | |
| strB | R primer | TGCGTCCGCCATCTGTGCA | 308. | |
| strB | 3′ probe | CCACGCAGTTCCGAGGCATT | 309. | |
| strB | 5′ probe | ACAAAGGTCGTCTCTGTCAGC | 310. | |

Results

All 40 targets were detected by real-time PCR with sensitivities ≤100 copies/reaction, and 36/40 were detected at 10 copies/reaction (Table 27). All targets had efficiencies >80% except for Entercoccus faecium. Since the Enterococcus faecium and Enterococcus faecalis targets use the same primers, this may indicate poor quality of the E. faecium gDNA rather than the primer set.

All 40 targets were detected by T2MR at concentrations ≤100 copies/reaction, and 39/40 targets were detected at 50 copies/reaction (Table 27). Pan Gram Positive and Pan Gram Negative channels had elevated signals in the negatives in both real-time and T2MR assays. However, positive and negative signals were distinctly separated in the T2MR assay and an elevated cutoff may allow for better discrimination in this assay.

Initial testing of the CTX-M universal target with gene variants from the CTX-M-2, CTX-M-8, and CTX-M-14 groups had 100% positive hit rates at 50 copies/reaction. The gene variant from the CTX-M-15 group had a 75% positive hit rate with this design at 50 copies/reaction and some optimization may be required to improve sensitivity.

TABLE 27
Real-time PCR and T2MR detection of T2AMR targets

| | Real-Time | Real-Time | | T2MR | | |
|---|---|---|---|---|---|---|
| | PCR | PCR | T2MR | Avg | T2MR | |
| | Sensitivity | Efficiency | gDNA | Signal | Stdev | T2MR |
| Channel | (cp/rxn) | (%) | (cp/rxn) | (ms) | (ms) | CV (%) |
| Pan Gram Positive | 10 | 126 | 50 | 496 | 49 | 10 |
| Pan Gram | 10 | 115 | 50 | 447 | 19 | 4 |
| Negative | | | | | | |
| Staphylococcus | 10 | 84 | 50 | 581 | 44 | 7 |
| aureus | | | | | | |
| Coagulase | 10 | 84 | 50 | 752 | 137 | 18 |
| negative | | | | | | |
| staphylococci | | | | | | |
| Enterococcus spp. | 10 | 81 | 50 | 468 | 39 | 8 |
| Enterococcus | 10 | 65 | 50 | 253 | 12 | 5 |
| faecium | | | | | | |
| Enterococcus | 10 | 81 | 50 | 203 | 15 | 8 |
| faecalis | | | | | | |
| Streptococcus | 10 | 90 | 50 | 334 | 47 | 14 |
| pneumoniae | | | | | | |
| Streptococcus | 10 | 84 | 50 | 474 | 38 | 8 |
| agalactiae | | | | | | |
| Streptococcus | 10 | 103 | 50 | 285 | 6 | 2 |
| pyogenes | | | | | | |
| Clostridium spp. | 10 | 94 | 50 | 358 | 107 | 30 |
| Mycobacterium spp. | 10 | 98 | 50 | 352 | 11 | 3 |
| Enterobacterales | 10 | 94 | 50 | 343 | 25 | 7 |
| Escherichia coli | 10 | 88 | 50 | 180 | 4 | 2 |
| Klebsiella | 10 | 87 | 50 | 519 | 11 | 2 |
| pneumoniae | | | | | | |
| Klebsiella | 10 | 81 | 50 | 534 | 8 | 1 |
| aerogenes | | | | | | |
| Enterobacter | 10 | 84 | 50 | 633 | 17 | 3 |
| cloacae complex | | | | | | |
| Citrobacter spp. | 10 | 95 | 50 | 568 | 92 | 16 |
| Serratia spp. | 10 | 85 | 50 | 326 | 12 | 4 |
| Proteus spp. | 10 | 92 | 50 | 448 | 43 | 10 |
| Acinetobacter | 10 | 93 | 50 | 120 | 2 | 2 |
| baumannii | | | | | | |
| Bacteroides spp. | 10 | 94 | 50 | 135 | 22 | 16 |
| Haemophilus | 10 | 80 | 50 | 510 | 146 | 29 |
| influenzae | | | | | | |
| Pseudomonas | 10 | 88 | 50 | 735 | 6 | 1 |
| aeruginosa | | | | | | |
| Stenotrophomonas | 10 | 82 | 50 | 576 | 4 | 1 |
| maltophilia | | | | | | |
| mecA | 10 | 95 | 50 | 382 | 204 | 53 |
| mecC | 10 | 85 | 50 | 491 | 31 | 6 |
| vanA/B | 10 | 81 | 50 | 297 | 16 | 5 |
| mefA/E | 10 | 82 | 50 | 793 | 81 | 10 |
| KPC | 100 | 89 | 50 | 515 | 169 | 33 |
| NDM | 100 | 88 | 50 | 456 | 34 | 8 |
| VIM | 10 | 85 | 50 | 592 | 33 | 6 |
| IMP | 100 | 86 | 100 | 491 | 18 | 4 |
| OXA-48 | 10 | 97 | 50 | 833 | 40 | 5 |
| OXA-23 | 10 | 117 | 50 | 225 | 34 | 15 |
| OXA-24/40 | 10 | 86 | 50 | 748 | 71 | 10 |
| CTX-M | 10 | 93 | 50 | 549 | 115 | 21 |
| AmpC | 10 | 94 | 50 | 605 | 76 | 13 |
| mcr-1 | 100 | 103 | 50 | 433 | 76 | 18 |
| strA/strB | 10 | 96 | 50 | 571 | 25 | 4 |

### CONCLUSIONS

Detection of ≤100 CFU/mL was demonstrated for forty (40) channels using the exemplary T2AMR panel described in this Example with both real-time PCR and T2MR detection methods.

SEQUENCE LISTING
Note: “K” is equivalent to a G or T mixed base. “I” indicates inosine.

| SEQ ID | |
|---|---|
| NO | Sequence |
| 1. | CCAGCTCGCACCGAATGTCT |
| 2. | CATCTTGTCCTGATGCGCGTGAGTCA |
| 3. | TTTCTGCCACCGCGCTGAC |
| 4. | GCAGCAAGAAAGCCCTTGAATG |
| 5. | ACATTTCCATAGCGACAGCACGGGCGGAAT |
| 6. | GGACTTTGGCCAAGCTTCTAAATTTGCGTC |
| 7. | CGTGCAGTCTCCACGCACT |
| 8. | TCGAATGCGCAGCACCGGGATAG |
| 9. | GGCTGTGTTTTTGGTGGCATCGATTATC |
| 10. | TCCCACTTAAAGACTTGGTGTTCATCC |
| 11. | ACGGGCCGGTAATGCGGATCTGGA |
| 12. | TATTCGCCAGAATCACAATCGCCACCTGT |
| 13. | CCGCGGCGAAATTAAGCTCAGCGA |
| 14. | CCAAACAGACCAATGCTGGAGTTAG |
| 15. | ATTCGTTGCACTATCGTTAACTGAATACA |
| 16. | CTGTACCGTCGGACTTTCCAGAC |
| 17. | GCGACCGGCAGGTTGATCT |
| 18. | CATGTCGAGATAGGAAGTGTGCTGC |
| 19. | CGGAACCATTCGCTAAACTC |
| 20. | AGGCGCAACTGTAAGTTACCG |
| 21. | GCTTAATTCTCAATCTATCCCCACGTAT |
| 22. | CTCCAGATAACGTAGTGGTTTGGCTG |
| 23. | CTTTCATGACGACCGCGTCGG |
| 24. | CTCTAGAAGGACTCTCATCGAGC |
| 25. | ATTTTAAAGGTAGATGCGGG |
| 26. | CGCCCTGTGATTTATGTTCA |
| 27. | GTTTTATGCACCCAGGAAGC |
| 28. | TCTGCTGCGGCCAGTCATA |
| 29. | GCGGCTGCCAGTTTTGATAA |
| 30. | GTGGCTAAGTGCAGCAGGC |
| 31. | CGTTCCACTAACACACAAGCTGATTCAG |
| 32. | ATCTCGGTTGATTTCTTTTCCTCGGG |
| 33. | ACGCTTTCCAATGTGCAGTACCAGTA |
| 34. | TGCGATCCAGACGAAACGTCTCATCG |
| 35. | GTGATACCACTTCACCTCGGGCAA |
| 36. | AATACATCGCGACGGCTTTCTGCC |
| 37. | AGAATTACCTTTGAAAGTCAGGC |
| 38. | GCTTCAAAGCCTGTCGGAATTGGTTT |
| 39. | GGGCATTTAACGACGAAACTGGCTA |
| 40. | GTGTTCGGTGAATATCCAAGGTACGC |
| 41. | ATGTTGGTCCCATTAACTCTGAAGAA |
| 42. | CACCTGTTTGAGGGTGGATAGCAGTA |
| 43. | GCAGGGCAAGCAGTATCATTAATCAC |
| 44. | AATTAAATCAGCACCAATCATTATCTTCTTCC |
| 45. | AAGCTGCTGACCAGCCAGCGTCTGA |
| 46. | CGGCGATTTGCTGATTTCGCTCG |
| 47. | TGCAGTGCTGCCATAACCATGAGTGA |
| 48. | AGCGCAGAAGTGGTCCTGCAACTTT |
| 49. | CAGTACGGAATCTTTCGTATTCATCAGGA |
| 50. | CAGTTCGGGAAGTGCAATACCTGCA |
| 51. | AATTGAGCAAGCGATTTCGGGCTGT |
| 52. | CGTTTAGAACGATGCCGCCATCCT |
| 53. | CCTTGGACGGAAATGTAGCTGGCA |
| 54. | AATCACATGGTTGACACCTGCTGTG |
| 55. | GGAAATCTAACGAGAGAGCATGCT |
| 56. | CGATGCGTGACACCCAGGC |
| 57. | CTGTCGAGATCAAGCCTGCCGA |
| 58. | ACAAATTGATTGCCCAGCTCGGT |
| 59. | AATCAGCGGCGCACGATCTTT |
| 60. | AATGCTCGCTGCACCGGTGGTAT |
| 61. | AAATCTGCAACGAGCTTTGGG |
| 62. | GTTTATAAGTGGGTAAACCGTGAATATC |
| 63. | TTCGTGTCACTTTAATTCACCAAGAT |
| 64. | AAAGCCATGCGTCTGACATCT |
| 65. | AAGCTCCAACATGAAGATGGCT |
| 66. | AGATGGCAAAGATATTCAACTAAC |
| 67. | AGTGCCATCTTGCAAATGGCGAT |
| 68. | TGCAATTGGTGTGTTAGTGGATCGTCATGATA |
| 69. | CAGTGGATGGTGGACGATCGGGT |
| 70. | TTGTGGTGATTTATCTGCGGGATACT |
| 71. | CGCCAGTTAATAGTTTGCGCAACG |
| 72. | AAAGCGGTTAGCTCCTTCGGTCCT |
| 73. | CGTTCAGTACAATGCGGCCGTTA |
| 74. | CTGCGGGAACGGTTATAACTGC |
| 75. | GCACCCGATATACTTTCTTTGCC |
| 76. | CGCCGACAATCAAATCATCCT |
| 77. | TTGACCAGTTCCGAGCAAATGGTA |
| 78. | GACAGTATCGATGTTCCAGCAGCT |
| 79. | GAGACGTTTTGGATACATGTGAAAGAAGGC |
| 80. | CGATGGTTCACGGGATTCTGCAATTC |
| 81. | CGT TTT CCA AAT CTG TAA CAG ACT GGG |
| 82. | AGG ACG TTG ATA GG TTG GAT GTG GA |
| 83. | GGT AGC TAT GTA GGG AAG GGA TAA ACG CTG A |
| 84. | GCG CTA AGG AGC TTA ACT TCT GTG TTC G |
| 85. | GAC GGT TGT CCC GGT TTA AGC A |
| 86. | GCT GGT ATC TTC GAC TGG TCT |
| 87. | AGG CTG GGT GTG TAA GCG TTG T |
| 88. | CAA GCA ATT CGG TTG GAT ATC CGT T |
| 89. | GGT AAT GAATTA CCT/i6diPr/TC TCT GCT GGTTTC TTC TT |
| 90. | ACC AGC ATC TTC/i6diPr/GC ATC TTC TGT AAA |
| 91. | GAA GTT ATG TTT/i6diPr/CT ATT CGA ATC GTG GTC CAGT |
| 92. | GTT GTA AAG CCA TGA TGC TCG TAA CCA |
| 93. | GGC ATG CCT GTT TGA GCG TC |
| 94. | GCT TAT TGA TAT GCT TAA GTT CAG CGG GT |
| 95. | TGA GGC TTG ACT ATA CAA CAC C |
| 96. | CTA AAA TGA ACA GAT AAA GTA AGA TTC AA |
| 97. | TTT TTT TTT TGA GGC TTG ACT ATA CAA CAC C |
| 98. | CTA AAA TGA ACA GAT AAA GTA AGA TTC AAT TTT TTT TT |
| 99. | AAA ACT TAT ATG ACT TCA AAT CCA GTT TT |
| 100. | TTT ACT CAA TAA AAG ATA ACA CCA CAG |
| 101. | ttt ttt ttt AAA ACT TAT ATG ACT TCA AAT CCA GTT TT |
| 102. | TTT ACT CAA TAA AAG ATA ACA CCA CAG Ttt ttt ttt t |
| 103. | TGG ATA AGT AAA AGC AAC TTG GTT |
| 104. | AAT GAA GAT TCA ACT CAA TAA GAA ACA ACA |
| 105. | ttt ttt ttt TGG ATA AGT AAA AGC AAC TTG GTT |
| 106. | AAT GAA GAT TCA ACT CAA TAA GAA ACA ACA ttt ttt ttt |
| 107. | TAC CAA GGC GCT TGA GAG AAC TC |
| 108. | CTG GTG TGT AGG TGA AGT C |
| 109. | TTT TTT TTT TAC CAA GGC GCT TGA GAG AAC TC |
| 110. | CTG GTG TGT AGG TGA AGT CTT TTT TTT T |
| 111. | GTG TGT TGT AGG GTG AAG TCG AC |
| 112. | CAC CTT GAA ATC ACA TAC CTG A |
| 113. | ttt ttt ttt GTG TGT TGT AGG GTG AAG TCG AC |
| 114. | CAC CTT GAA ATC ACA TAC CTG Att ttt ttt t |
| 115. | CCA TTT GAA GTT GTT TAT TAT GC |
| 116. | GGG AAA TGA TTA ATT ATG CAT TAA ATC |
| 117. | TTT TTT TTT CCA TTT GAA GTT GTT TAT TAT GC |
| 118. | GGG AAA TGA TTA ATT ATG CAT TAA ATC TTT TTT TTT |
| 119. | TT TTT CAG ATT TAG GAT TAG TTG ATT |
| 120. | GAT CCG TAT TGG TTA TAT CAT C |
| 121. | TT TTT TTT TTT TTT CAG ATT TAG GAT TAG TTG ATT |
| 122. | GAT CCG TAT TGG TTA TAT CAT CTT TTT TTT T |
| 123. | TTT TTT TTT TGG AAT AAT ACG CCG ACC AGC |
| 124. | AAG GAT CTA TTT CAG TAT GAT GCA GTT TTT TTT T |
| 125. | TGCCGAAGCGTTTTCCAAATCTGTAACAGACTGGGCTGATTGAATCTTACTTTAT |
| | CTGTTCATTTTAGCTAGAGGTATAACTAAATCAAGTTGTCTTGCATATTTAAGAAT |
| | CGATTGATGCTTTATATACAACTGCTTGGGTGTTGTATAGTCAAGCCTCACGAGC |
| | AATTAGTATTGGTCAGCTTCACATATCACTATGC |
| 126. | GCATGGGAACAGGTGTATCCTTCTCGCTATCGCCACCACACTGGGTGTTGTTTCT |
| | TATTGAGTTGAATCTTCATTCACTCAAAACTGGATTGAAGTTTGAATCAAAATAAC |
| | CAAGTTGCTTTTACTTATCCATTCTTTGGTTAAGTCCTCGACCGATTAGTATTGGT |
| | CCGCTCCAACTATCACTAGCCTTCCACTTCCAA |
| 127. | GCATGGTTACAGGTGTATCCTTCTCGCTATCGCCACCACACTGTGGTGTTATCTT |
| | TTATTGAGTAAATTTTGTTCACTCAAAACTGGATTTGAAGTCATATAAGTTTTTTTC |
| | CGAGTTCTTTTCTTTTAACCTATTGGTTAAGTCCTCGATCGATTAGTATCAGTCCG |
| | CTCCATACATCACTGTACTTCCACTCCTGACC |
| 128. | CAGCTCCATCCGCAGGGACTTCACCTACACACCAGCGTGCCTTCTCCCGAAGTT |
| | ACGGCACCATTTTGCCTAGTTCCTTCACCCGAGTTCTCTCAAGCGCCTTGGTATT |
| | CTCTACCTGACCACCTGTGTCGGTTTGGGGTACGATTTGATGTTACCTGATGCTT |
| | AGAGGCTTTTCCTGGAAGCAGGGCATTTGTTACTTC |
| 129. | CGCTTGGGCTTACGTCTATCCGGATTCAGGTATGTGATTTCAAGGTGTTTTGCGG |
| | TTCATGCGAACTTTCGGTTCGTCGACTTCACCTTACAACACACAATCGTCAGATT |
| | GTTTGGGTGTTATATGGTCAAGCCTCACGGGCAATTAGTACTGGTTAGCTCAACG |
| | CCTC |
| 130. | TTTACCACTAACACCATAGAAATTATAACGGTCAATGCCATGATTTAATGCATAAT |
| | TAATCATTTCCCATTGCACTGCATAACTTCCGGCAAAATGACGGAATGCATTTGAT |
| | GTACCACCAGCATAATAAACAACTTCAAATGGGTTGATA |
| 131. | TGTGATTTAAACAAGTTTACTAAGGCATCATTTTTGTCGCGACCTTCAAATGGCAC |
| | GATATCTTTATCATATAGATGATATAACCAATACGGATCTAATTTAACATATAAACA |
| | TTGATGTTGCTGTAAATATTTATCTAACTCTTTTAAATAATAATCAACTAATCCTAA |
| | ATCTGAAAAATCCATT |
| 132. | GCA TTA ATC GAC GGT ATG GTT GAC C |
| 133. | CCT GCT GAA ACA GGT TTT CCC ACA TA |
| 134. | AGT GAT GAT GAG TTG TTT GCC AGT G |
| 135. | TGA ATT GTC GCC GCG TGA CCA G |
| 136. | ACC CAG CGG TTT GAG GGA GAA AC |
| 137. | AAA GTT TGA AGA TAT ACG TGG TGG ACG TTA |
| 138. | CGC ACG CGC AAG ATG GAA ACG |
| 139. | AAG TTC AGC GGG TAT TCC TAC CT |
| 140. | AGC TTT TTG TTG TCT CGC AAC ACT CGC |
| 141. | CTA CCA AAC ACA ATG TGT TTG AGA AG |
| 142. | CCT GAT TTG AGG TCA AAC TTA AAG ACG TCT G |
| 143. | AGT CCT ACC TGA TTT GAG GTC NitInd AA |
| 144. | CCG NitInd GG GTT TGA GGG AGA AAT |
| 145. | AAA GTT ATG AAATAA ATT GTG GTG GCC ACT AGC |
| 146. | ACC CGG GGGTTT GAG GGA GAA A |
| 147. | AGT CCT ACC TGA TTT GAG GTC GAA |
| 148. | CCG AGG GTT TGA GGG AGA AAT |
| 149. | GGA AGG GAT CAG GTG GTT CAC TCT T |
| 150. | AAA ACT TAT GTG ACT TCA AAT CCA GTT TT |
| 151. | TTT ACT CAA TAA AAG ATA ACA CCA CAG T |
| 152. | ttt ttt ttt AAA ACT TAT GTG ACT TCA AAT CCA GTT TT |
| 153. | TCT GAC GAT TGT GTG TTG TAA GG |
| 154. | GGA TAG ACG TAA GCC CAA GC |
| 155. | ttt ttt ttt TCT GAC GAT TGT GTG TTG TAA GG |
| 156. | GGA TAG ACG TAA GCC CAA GCtt ttt ttt t |
| 157. | GCA TGG TTA CAG GTG TAT CCT TCT CGC TAT CGC CAC CAC ACT GTG GTG |
| | TTA TCT TTT ATT GAG TAA ATT TTG TTC ACT CAA AAC TGG ATT TGA AGT |
| | CAT ATA AGT TTT TTT CCG AGT TCT TTT CTT TTA ACC TAT TGG TTA AGT |
| | CCT CGA TCG ATT AGT ATC AGT CCG CTC CAT ACA TCA CTG TAC TTC CAC |
| | TCC TGA |
| 158. | GGC ATG CCT GTT TGA GCG T |
| 159. | GGG CAT GCC TGT TTG AGC GT |
| 160. | CTA CCT GAT TTG AGG CGA CAA CAA AAC |
| 161. | CCG CGA AGA TTG GTG AGA AGA CAT |
| 162. | CCT ACC TGA TTT GAG GGC GAA ATG TC |
| 163. | GGA GCA ACG CCT AAC CGG G |
| 164. | GTC CTA CCT GAT TTG AGG GGA AAA AGC |
| 165. | AAC AAA TCC ACC AAC GGT GAG AAG ATA T |
| 166. | CGT AGA CTT CGC TGC GGA T |
| 167. | GCG TAG ACT TCG CTG CGG AT |
| 168. | CTG GGC GGT GAG AAG AAA TC |
| 169. | GCG TAG ACT TCG CTG CTG GAA |
| 170. | CCG TGC GGT GAG AAG AAA TC |
| 171. | TCC TAC CTG ATT TGA GGA AAT AGC ATG G |
| 172. | ATT TAG CGG ATG CAA AAC CAC C |
| 173. | CTCCTACGGGAGGCAGCAGT |
| 174. | GTATTACCGCGGCTGCTGGCA |
| 175. | CTGACGGAGCAACGCCGCGTGAGTGA |
| 176. | CTAACCAGAAAGCCACGGCTAACTACG |
| 177. | CTGATCCAGCCATGCCGCGTGTATGA |
| 178. | CCGCAGAAGAAGCACCGGCTAACTCCG |
| 179. | CACATTOTTTTATCACGTAACGTTGGTGT |
| 180. | CCAGGCATTACCATTTCAGTACCTTCTGGTAA |
| 181. | CCAGTTACGTCAGTAGTACGGAA |
| 182. | TTTGATTTGACCACGTTCAACAC |
| 183. | AGATTGTTCAAGGACATAATCAGGTGA |
| 184. | GGTAAATGACCTTTTCTCGCCCTTC |
| 185. | CTCAGGTGTGCTGGTTATTCA |
| 186. | GCCCTGATCGGATTGGAGAA |
| 187. | CCTCGGGCAATGGCGCAAAC |
| 188. | ATCGCGACGGCTTTCTGCCTTA |
| 189. | TATTCATCAGGAAGTCGAGCCGGA |
| 190. | AAGATCAACACGGGCAAGCCCTCT |
| 191. | ATGTGGGTCCAATTAATTCTGACGAG |
| 192. | CTCCAGTTTTTGGTTGTAATGCTGTA |
| 193. | GGGCGCAGCTGGTGACAT |
| 194. | AAGATCGTGCGCCGCTGATT |
| 195. | GCAGTTATAACCGTTCCCGCAG |
| 196. | TAACGGCCGCATTGTACTGAACG |
| 197. | AAAGGCTATAAAGATGATGCAG |
| 198. | GAGTATTTATAACAACATGAAAAATGATT |
| 199. | GGCTTAGAACGCCTCTATGAT |
| 200. | AGAGTACAAGAAAGTATTTATAAACATATGA |
| 201. | CACTCGTACCGTCTCTGAGGGTGTTGCA |
| 202. | CGTAGATTCACGACCKGGACCAGGGCCT |
| 203. | ACAATGGTGTTATTAAAAGAGGCGTGAATGTG |
| 204. | AACCGTAGTCTAAAGCGGCTTTACCAGCAACT |
| 205. | TATCCGATTATTATGCCAGCCATGTATTCGA |
| 206. | CCAGTGGGTGTAGTGTGTCACGTTGAGC |
| 207. | ACTGCTTTGTATGCTTCTTTGGGAAGGTATTC |
| 208. | CAGCTTTTCATGCCGTTGGCTATCAG |
| 209. | CGCTGGCGCATGACGAGAAG |
| 210. | GCTTGCGGAGATGCCGTCATAGGTACCGGCTT |
| 211. | TAGATGAAGAACCATTTCCAGTCCATGGAAAC |
| 212. | CGTTGGCGATCAGATGCAG |
| 213. | GTTTGTAGTACATGAAGCACCTGAGATAACATC |
| 214. | CGGAAAAGAAATCAAAGTAACTGCTAAAAGC |
| 215. | ACCACGATATATTAGATGGAAGCGAA |
| 216. | GGGAGAATTAGGAGTAGACGTAGTATT |
| 217. | TGATCCCGTTCTGGATGGTGA |
| 218. | TTCCACATAATCCAGATGGAACCGACT |
| 219. | GAATCTGAATCACCGCGG |
| 220. | CCAGGATAGCGATGATAATCAC |
| 221. | TGAGGGAAAGGCGAAAAGAACCTTGGC |
| 222. | ATGGGTAGATCACCGGGTTTCGGGTCTATA |
| 223. | TTGTACGTACACGGTTTCAGGTTCTT |
| 224. | CGCCCGGTTAAGACTCGGTTTCC |
| 225. | TGTGGTGGCGATGGCGAGAAGGATACA |
| 226. | GAAGCCCACCTCAAGATGAGATTTCC |
| 227. | CTTTATCAACGTAACGGCATTGGTCAATAC |
| 228. | CGCATAGGAGGGAAATGGTTATTACTTAAAC |
| 229. | GGAAAACATCTGGCTTGTGTGTTTTTAACC |
| 230. | GGTTGTCATCATTGATGTTCCCGC |
| 231. | GCCGTCAGTCAGCAGATGCAG |
| 232. | GATCCCCTGCTCGGTCAGTAAT |
| 233. | CGGCGGACTGGGTACGACATA |
| 234. | GCGAAAAGCTCTTTGCCTACCAG |
| 235. | TGATCCCGGTGTTGGCGATCGTG |
| 236. | TTCATGATTCCGGTGACGATCAGCGCC |
| 237. | TGCGCTTCGATAAACAGGTTCAGCGG |
| 238. | CCACAGCACCTGGCACACCAG |
| 239. | CTGCATCCGCGTGCCAATATCG |
| 240. | AGCATTTCCGGTTTGAAGAAATGCCC |
| 241. | CGGCATCATTCAGCGGGT |
| 242. | GGGCTTTCAGGCGGCTTTC |
| 243. | TGGTCAGGTGGTCAGCGAAGAGGTCGG |
| 244. | CGTAGTCGGAGTCGCTGTTCTC |
| 245. | GGTGACGACGACCTTGCGGGCTTT |
| 246. | TCTGGGCCACGCGGCCGG |
| 247. | CCGCAGAAGAAGCACCGGCTAACTCCGT |
| 248. | CTAACCAGAAAGCCACGGCTAACTACGT |
| 249. | TCTGACGGAGCAACGCCGCGTGAGTGA |
| 250. | CGCAGAGGTTTATATGTCAATTCCAGCTA |
| 251. | TCTTGATAAGCGGCGACCAGCTTATT |
| 252. | CAATATTGGATGCTTGTAAAGTTTGAATT |
| 253. | TTCAACGCCGGGCGTACCTAACGT |
| 254. | CGCCGTARCCGTTGCTGATG |
| 255. | CGATCACCACGTCTAAACGTACTGCCGA |
| 256. | CAGTACCAGTTCGACTTTGGTCTGC |
| 257. | ACTGGTGGCCTGAAATACGATGCGAACAAC |
| 258. | CCTGAAGAAGTCCGGTGCCTTCGTA |
| 259. | AGCCATAAAGTCCATGATGACGACAAC |
| 260. | CCAAAGTAGAATGGTACTTTCATTGCA |
| 261. | TCGCGCCCACCAGGGTTAAAC |
| 262. | GCTACTGGTCGTGTTGAACGTGGTCAAATCA |
| 263. | ACAACGCCTGAWCCTACAGTACGTCCA |
| 264. | AGATGTGTCATGTAAACCGAT |
| 265. | ATACTTCTGCTTTGAATTCAGTATG |
| 266. | GTTGTGGGTACCGATGATTTCAACTTC |
| 267. | AGGTAAAAAGTATACTTCAGCTTTGAATTTAGTGTG |
| 268. | TGCTTGATTCTTCTTGCATACCGATG |
| 269. | AAAACTTGACCACGTTGGACAT |
| 270. | CTGATCATGCTGTCGCTGTGGATC |
| 271. | GGTGATGGTCAGCACCGGGAA |
| 272. | CTGTATTCGATGCCCGACAGTGGTGGGAA |
| 273. | CGGCATCTGCATGAGCTTCATGCT |
| 274. | GGCATTGGTCCGTCTGTTGATGCAACTACAAGG |
| 275. | GGCATTGGACCGTCAGTTGAAGCAACTACAAGG |
| 276. | TGGCAAAAGAAAAATACGATCGTAGTAAACC |
| 277. | TCACTGGTGCTGCTCAAATGG |
| 278. | ACGTTAACATTGGTACAATTGGACACG |
| 279. | CCGTCCGGGTGATCCAGGTGATCATGGTAAAGG |
| 280. | GTTGGCAGTAATGCTGCCGCGATCTGGCAT |
| 281. | TGGTCAATGGCATATTGAGCAAC |
| 282. | CTTGCAAATGGCGCATAGAATCG |
| 283. | CATATTTTGTGTAGAGTACTGTGCAACTT |
| 284. | ATTCCAAGTATTAGCAGGTCCATAA |
| 285. | CGTTTTCCTATGTGCAGTACCAGTAAGGTTATGGC |
| 286. | CGTTTTGCTATGTGCAGTACCAGTAAGGTGATGGC |
| 287. | GGTGAGGTGGTGTCGCGCGGGTCGCCTGGGAT |
| 288. | GGTGAGGTGGTGTCTCTCGGGTCGCCTGGGAT |
| 289. | GCTTTCACTTTTGTTCAGCACCGCGGCC |
| 290. | CGTTTCACTCTGCTTAAGCACCGCCGCG |
| 291. | CGTTTCACTTTGCTTGAGCACCGCCGT |
| 292. | GGCGGTGTTTAACGTCGGCTCGGTACG |
| 293. | GGCGGTATTCAGCGTAGGTTCAGTGCG |
| 294. | CGCGGTGTTGAGCGTCGGCTCAGTACG |
| 295. | AAATGCCCTACAGACCGACCAAG |
| 296. | TGAACGCCACCACAGGCAGTAAA |
| 297. | CCAATGATACGCATGATAAACGCT |
| 298. | CAAGACTTGCCACGATCAAGC |
| 299. | GGTGAGGCAATGGCATTGTCAG |
| 300. | GTAATCGGTTATGTGCAAGGTCATCGG |
| 301. | CTTGCAAGCTCTTGATATACTGG |
| 302. | ACTTCTTGTACTGGTGTAATTTTAAGGG |
| 303. | ATCCCTGCATGCCGAACTTCATG |
| 304. | GCTCTGCTTCATCTGGCGCTG |
| 305. | AGACCCGTGCATTGAAGAGTT |
| 306. | CCAGTTCTCTTCGGCGTTAGC |
| 307. | GCGGCGATTATAGCCGGTCAAATGATGA |
| 308. | TGCGTCCGCCATCTGTGCA |
| 309. | CCACGCAGTTCCGAGGCATT |
| 310. | ACAAAGGTCGTCTCTGTCAGC |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for detecting the presence of a pathogen in a biological sample, the method comprising:
(a) amplifying in a biological sample or a fraction thereof one or more pathogen target nucleic acids in a multiplexed amplification reaction, wherein the multiplexed amplification reaction is configured to amplify a panel comprising at least 28 pathogen target nucleic acids, wherein the panel comprises (i) one or more genus-level target nucleic acids, (ii) one or more Gram positive bacterial target nucleic acids, (iii) one or more Gram negative bacterial target nucleic acids, and (iv) one or more resistance gene target nucleic acids; and
(b) detecting the one or more amplified pathogen target nucleic acids to determine whether the pathogen is present in the biological sample,
wherein the method has a percent coverage of greater than or equal to 90% of pathogen species associated with infections of the sample.

2. The method of claim 1, wherein step (a) comprises amplifying the one or more pathogen target nucleic acids in a lysate produced by lysing cells in the biological sample.

3. A method for detecting the presence of a pathogen in a biological sample, the method comprising:
(a) providing a biological sample and optionally dividing the sample into one or more portions;
(b) lysing pathogen cells in the biological sample or the one or more portions thereof to form one or more lysates;
(c) amplifying, in the one or more lysates, one or more pathogen target nucleic acids in a multiplexed amplification reaction to form one or more amplified lysates, wherein the multiplexed amplification reaction is configured to amplify a panel comprising at least 28 pathogen target nucleic acids, wherein the panel comprises (i) one or more genus-level target nucleic acids, (ii) one or more Gram positive bacterial target nucleic acids, (iii) one or more Gram negative bacterial target nucleic acids, and (iv) one or more resistance gene target nucleic acids;
(d) preparing a plurality of assay samples by contacting the one or more amplified lysates with a plurality of populations of magnetic particles, wherein each population of magnetic particles has binding moieties characteristic of one or more of the pathogen target nucleic acids on its surface, the binding moieties operative to alter aggregation of the magnetic particles in the presence of an amplified pathogen target nucleic acid;
(e) providing each assay sample in a detection tube within a device, the device comprising a support defining a well for holding the detection tube comprising the assay sample, and having an RF coil configured to detect a signal produced by exposing the mixture to a bias magnetic field created using one or more magnets and an RF pulse sequence;
(f) exposing each assay sample to a bias magnetic field and an RF pulse sequence;
(g) following step (f), measuring the signal produced by each assay sample; and
(h) on the basis of the result of step (g), detecting whether one or more of the pathogens is present in the biological sample, wherein the method has a percent coverage of greater than or equal to 90% of pathogen species associated with infections of the sample.

4. The method of claim 2 or 3, wherein the lysate has at least about a 2:1, a 5:1, a 10:1, a 20:1, a 40:1, or a 60:1 higher concentration of cell debris relative to the biological sample.

5. The method of claim 4, wherein the cell debris is solid material.

6. The method of any one of claims 1-5, wherein the biological sample has a volume of about 0.1 mL to about 5 mL.

7. The method of claim 6, wherein the biological sample has a volume of about 2 mL.

8. The method of any one of claims 1-7, wherein the biological sample is selected from the group consisting of blood, a bloody fluid, a tissue sample, bronchiolar lavage (BAL), urine, cerebrospinal fluid (CSF), synovial fluid (SF), and sputum.

9. The method of claim 8, wherein the blood is whole blood, a crude blood lysate, serum, or plasma.

10. The method of claim 9, wherein the whole blood is ethylenediaminetetraacetic acid (EDTA) whole blood, sodium citrate whole blood, sodium heparin whole blood, lithium heparin whole blood, or potassium oxylate/sodium fluoride whole blood.

11. The method of claim 8, wherein the bloody fluid is wound exudate, wound aspirate, phlegm, or bile.

12. The method of claim 8, wherein the tissue sample is a tissue sample from a transplant, a tissue biopsy (e.g., a skin biopsy, muscle biopsy, or lymph node biopsy), a homogenized tissue sample, or bone.

13. The method of claim 8, wherein the biological sample is urine or BAL.

14. The method of any one of claims 1-7, wherein the biological sample is a swab.

15. A method for detecting the presence of a pathogen in a whole blood sample, the method comprising:
(a) amplifying, in one or more lysates produced from a whole blood sample, one or more pathogen target nucleic acids in a multiplexed amplification reaction, wherein the multiplexed amplification reaction is configured to amplify a panel comprising at least 28 pathogen target nucleic acids, wherein the panel comprises (i) one or more genus-level target nucleic acids, (ii) one or more Gram positive bacterial target nucleic acids, (iii) one or more Gram negative bacterial target nucleic acids, and (iv) one or more resistance gene target nucleic acids, wherein each of the one or more lysates is produced by:
(i) contacting the whole blood sample or a portion thereof with an erythrocyte lysis agent, thereby lysing red blood cells;
(ii) centrifuging the product of step (i) to form a supernatant and a pellet;
(iii) discarding some or all of the supernatant of step (ii) and resuspending the pellet to form an extract, optionally washing the pellet one or more times prior to resuspending the pellet; and
(iv) lysing the remaining cells in the extract of step (iii) to form the lysate, the lysate containing both subject cell nucleic acid and pathogen nucleic acid;
(b) detecting one or more amplified pathogen target nucleic acids in the one or more lysates, thereby detecting the presence of the pathogen in the sample, wherein the method has a percent coverage of greater than or equal to 90% of pathogen species associated with blood infections.

16. The method of any one of claims 1-15, wherein the panel further comprises (v) one or more pan-level target nucleic acids.

17. The method of any one of claims 1-16, wherein the panel further comprises (vi) one or more fungal target nucleic acids.

18. The method of any one of claims 1-17, wherein the method has a percent coverage of greater than or equal to 95%, 96%, 97%, 98%, or 99% of pathogen species associated with infections of the sample.

19. The method of claim 18, wherein the method has a percent coverage of greater than or equal to 99% of pathogen species associated with infections of the sample.

20. The method of any one of claims 1-19, wherein the panel comprises at least two subpanels.

21. The method of claim 20, wherein the panel comprises at least four subpanels.

22. The method of claim 20 or 21, wherein the panel comprises five subpanels.

23. The method of any one of claims 20-22, wherein each subpanel comprises at least six pathogen target nucleic acids.

24. The method of claim 23, wherein each subpanel comprises nine pathogen target nucleic acids.

25. The method of claim 23, wherein each subpanel comprises fourteen pathogen target nucleic acids.

26. The method of any one of claims 20-25, wherein each subpanel comprises an internal control channel.

27. The method of any one of claims 1-26, wherein the panel comprises at least 36 pathogen target nucleic acids.

28. The method of any one of claims 1-27, wherein the panel comprises at least 40 pathogen target nucleic acids.

29. The method of claim 27 or 28, wherein the panel comprises 45 pathogen target nucleic acids.

30. The method of claim 29, wherein the 45 pathogen target nucleic acids are split between five subpanels.

31. The method of any one of claim 1-30, wherein the one or more genus-level target nucleic acids are characteristic of a genus selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp.

32. The method of claim 31, wherein the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, or all twenty-two genus-level target nucleic acids selected from the group consisting of Acinetobacter spp., anaerobes, Bacteroides spp., Citrobacter spp., Clostridium spp., Corynebacterium spp., Enterobacter spp., Enterobacter cloacae complex, Enterobacteriaceae, Enterococcus spp., Mycobacterium spp., Neisseria spp., Proteus spp., Salmonella spp., Serratia spp. Staphylococcus spp., coagulase negative Staphylococcus spp., Streptococcus spp., Viridans group Streptococcus, Aspergillus spp., Candida spp., and Cryptococcus spp.

33. The method of claim 31 or 32, wherein the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen genus-level target nucleic acids selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp.

34. The method of any one of claims 31-33, wherein:
(i) the genus-level target nucleic acid characteristic of Enterobacteriaceae is characteristic of Klebsiella spp., Enterobacter spp., Citrobacter spp., Serratia spp., Proteus spp., and/or Morganella spp.;
(ii) the genus-level target nucleic acid characteristic of coagulase negative Staphylococcus spp. is characteristic of S. epidermidis, S. haemolyticus, S. lugdunensis, and/or S. hominis;
(iii) the genus-level target nucleic acid characteristic of Viridans group Streptococcus is characteristic of S. anginosus, S. mitis, and/or S. oralis; and/or
(iv) the genus-level target nucleic acid characteristic of anaerobes is characteristic of Clostridium spp. and/or Bacteroides spp.

35. The method of any one of claims 31-33, wherein the genus-level target nucleic acid is characteristic of Staphylococcus spp., and the Staphylococcus spp. target nucleic acid is amplified in the presence of a forward primer comprising the nucleotide sequence of CACATTCTTTTATCACGTAACGTTGGTGT (SEQ ID NO: 179) and a reverse primer comprising the nucleotide sequence of CCAGGCATTACCATTTCAGTACCTTCTGGTAA (SEQ ID NO: 180) to produce a Staphylococcus spp. amplicon.

36. The method of claim 35, wherein a probe pair comprising a 5′ probe comprising the nucleotide sequence of CCAGTTACGTCAGTAGTACGGAA (SEQ ID NO: 181) and a 3′ probe comprising the nucleotide sequence of TTTGATTTGACCACGTTCAACAC (SEQ ID NO: 182) is used for detection of the Staphylococcus spp. amplicon.

37. The method of any one of claims 31-33, wherein the genus-level target nucleic acid is characteristic of Candida spp., and the Candida spp. target nucleic acid is amplified in the presence of a forward primer comprising a nucleotide sequence selected from the group consisting of GGCATGCCTGTTTGAGCGTC (SEQ ID NO: 93), GGCATGCCTGTTTGAGCGT (SEQ ID NO: 157), and GGGCATGCCTGTTTGAGCGT (SEQ ID NO: 159), and a reverse primer comprising the nucleotide sequence of GCTTATTGATATGCTTAAGTTCAGCGGGT (SEQ ID NO: 94) to produce a Candida spp. amplicon.

38. The method of any one of claim 1-37, wherein the one or more Gram positive bacterial target nucleic acids are selected from the group consisting of E. faecium, E. faecalis, S. aureus, S. pneumoniae, S. pyogenes, and S. agalactiae.

39. The method of claim 38, wherein the panel comprises at least two, at least three, at least four, at least five, or all six Gram positive bacterial target nucleic acids selected from the group consisting of E. faecium, E. faecalis, S. aureus, S. pneumoniae, S. pyogenes, and S. agalactiae.

40. The method of claim 38 or 39, wherein the one or more Gram positive bacterial target nucleic acids is amplified in the presence of a forward primer and a reverse primer set forth in Table 3.

41. The method of claim 40, wherein the one or more Gram positive bacterial target nucleic acid amplicons is detected using a 5′ capture probe and a 3′ capture probe set forth in Table 4 or Table 26.

42. The method of any one of claims 1-41, wherein the one or more Gram negative bacterial target nucleic acids are selected from the group consisting of A. baumannii, E. coli, H. influenzae, K. pneumoniae, P. aeruginosa, S. marcescens, P. mirabilis, and S. maltophilia.

43. The method of claim 40, wherein the panel comprises at least two, at least three, at least four, at least five, at least six, at least seven, or all eight Gram negative bacterial target nucleic acids selected from the group consisting of A. baumannii, E. coli, H. influenzae, K. pneumoniae, P. aeruginosa, S. marcescens, P. mirabilis, and S. maltophilia.

44. The method of claim 42 or 43, wherein the one or more Gram negative bacterial target nucleic acids is amplified in the presence of a forward primer and a reverse primer set forth in Table 3.

45. The method of claim 44, wherein the one or more Gram negative bacterial target nucleic acid amplicons is detected using a 5′ capture probe and a 3′ capture probe set forth in Table 4.

46. The method of any one of claims 1-45, wherein the one or more resistance gene target nucleic acids are selected from the group consisting of mecA, mecC, mefA, mefE, mcr-1, vanA, vanB, ermA, ermB, KPC, NDM, VIM, IMP, OXA-23-like, OXA-48-like, SHV, CMY, DHA, CTX-M, TEM, FKS, PDR1, and ERG11.

47. The method of claim 46, wherein the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, or all twenty-three resistance gene target nucleic acids selected from the group consisting of mecA, mecC, mefA, mefE, mcr-1, vanA, vanB, ermA, ermB, KPC, NDM, VIM, IMP, OXA-23-like, OXA-48-like, SHV, CMY, DHA, CTX-M, TEM, FKS, PDR1, and ERG11.

48. The method of claim 46 or 47, wherein the panel comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, or all twenty-three resistance gene target nucleic acids selected from the group consisting of mecA, mecC, mefA, mefE, vanA, vanB, ermA, ermB, KPC, NDM, VIM, IMP, OXA-23-like, OXA-48-like, SHV, CMY, DHA, CTX-M 14, CTX-M 15, TEM, FKS, PDR1, and ERG11.

49. The method of any one of claims 46-48, wherein the resistance target nucleic acid is characteristic of mecA and mecC; mefA and mefE; vanA and vanB; ermA and ermB; NDM, VIM, and IMP;
CMY and DHA; or CTX-M.

50. The method of any one of claims 46-49, wherein the resistance target nucleic acid characteristic of CTX-M is a universal CTX-M target nucleic acid.

51. The method of claim 50, wherein the universal CTX-M target nucleic acid is characteristic of CTX-M-2, CTX-M-8, CTX-M-14, and CTX-M-15.

52. The method of claim 50 or 51, wherein the universal CTX-M target nucleic acid is amplified in the presence of (i) a first degenerate forward primer comprising the nucleotide sequence of CGTTTTCCIATGTGCAGTACCAGTAAGGTTATGGC (SEQ ID NO: 285) and a second degenerate forward primer comprising the nucleotide sequence of CGTTTTGCIATGTGCAGTACCAGTAAGGTGATGGC (SEQ ID NO: 286) and (ii) a first degenerate reverse primer comprising the nucleotide sequence of GGTGAGGTGGTGTCGCGCGGGTCGCCIGGGAT (SEQ ID NO: 287) and a second degenerate reverse primer comprising the nucleotide sequence of GGTGAGGTGGTGTCTCTCGGGTCGCCIGGGAT (SEQ ID NO: 288).

53. The method of any one of claims 50-52, wherein a plurality of probes comprising (i) a plurality of 5′ degenerate probes selected from GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a plurality of 3′ degenerate probes selected from GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291) are used for detection of the universal CTX-M amplicon.

54. The method of claim 53, wherein a first population of 5′ degenerate probes comprising the nucleotide sequences of GGCGGTGTTTAACGTCGGCTCGGTACG (SEQ ID NO: 292), GGCGGTATTCAGCGTAGGTTCAGTGCG (SEQ ID NO: 293), and CGCGGTGTTGAGCGTCGGCTCAGTACG (SEQ ID NO: 294) and (ii) a second population of 3′ degenerate probes comprising the nucleotide sequences of GCTTTCACTTTTCTTCAGCACCGCGGCC (SEQ ID NO: 289), CGTTTCACTCTGCTTAAGCACCGCCGCG (SEQ ID NO: 290), and CGTTTCACTTTGCTTGAGCACCGCCGT (SEQ ID NO: 291), are used for detection of the universal CTX-M amplicon.

55. The method of any one of claims 46-54, wherein the one or more resistance gene target nucleic acids is amplified in the presence of a forward primer and a reverse set forth in Table 10, Table 12, Table 14, Table 16, or Table 26.

56. The method of claim 55, wherein the one or more resistance gene target nucleic acid amplicons is detected using a 5′ capture probe and a 3′ capture probe set forth in Table 11, Table 13, Table 15, Table 17, or Table 26.

57. The method of any one of claims 47-56, wherein the resistance gene target nucleic acid is OXA-23-like, and the OXA-23-like target nucleic acid is amplified in the presence of a forward primer comprising the nucleotide sequence of AGATTGTTCAAGGACATAATCAGGTGA (SEQ ID NO: 183) and a reverse primer comprising the nucleotide sequence of GGTAAATGACCTTTTCTCGCCCTTC (SEQ ID NO: 184) to produce a OXA-23-like amplicon.

58. The method of claim 57, wherein a probe pair comprising a 5′ probe comprising the nucleotide sequence of CTCAGGTGTGCTGGTTATTCA (SEQ ID NO: 185) and a 3′ probe comprising the nucleotide sequence of GCCCTGATCGGATTGGAGAA (SEQ ID NO: 186) is used for detection of the OXA-23-like amplicon.

59. The method of any one of claims 16-58, wherein the one or more pan-level target nucleic acids are selected from the group consisting of Pan-Bacterial, Pan-Gram positive, Pan-Gram negative, and Pan-Fungal.

60. The method of claim 59, wherein the panel comprises at least two, at least three, or all four pan-level target nucleic acids selected from the group consisting of Pan-Bacterial, Pan-Gram positive, Pan-Gram negative, and Pan-Fungal.

61. The method of claim 59 or 60, wherein the Pan-Bacterial target nucleic acid is amplified in the presence of a forward primer comprising the nucleotide sequence of CTCCTACGGGAGGCAGCAGT (SEQ ID NO: 173) and a reverse primer comprising the nucleotide sequence of GTATTACCGCGGCTGCTGGCA (SEQ ID NO: 174) to produce a Pan-Bacteria amplicon.

62. The method of claim 44, wherein a probe pair comprising a 5′ probe comprising the nucleotide sequence of CTGACGGAGCAACGCCGCGTGAGTGA (SEQ ID NO: 175) and a 3′ probe comprising the nucleotide sequence of CTAACCAGAAAGCCACGGCTAACTACG (SEQ ID NO: 176) is used to detect the presence of Gram positive bacteria.

63. The method of claim 44, wherein a probe pair comprising a 5′ probe comprising the nucleotide sequence of CTGATCCAGCCATGCCGCGTGTATGA (SEQ ID NO: 177) and a 3′ probe comprising the nucleotide sequence of CCGCAGAAGAAGCACCGGCTAACTCCG (SEQ ID NO: 178) is used to detect the presence of Gram negative bacteria.

64. The method of any one of claims 17-63, wherein the one or more fungal target nucleic acids are selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii.

65. The method of claim 64, wherein the panel comprises at least two, at least three, at least four, at least five, at least six, at least seven, at least eight, at least nine, at least ten, or all eleven fungal target nucleic acids selected from the group consisting of C. albicans, C. tropicalis, C. dublinensis, C. parapsilosis, C. krusei, C. glabrata, C. auris, C. lusitaniae, C. haemulonii, C. duobushaemulonii, and C. pseudohaemulonii.

66. The method of claim 64 or 65, wherein the one or more fungal target nucleic acids is amplified in the presence of a forward primer and a reverse primer set forth in Table 7.

67. The method of claim 66, wherein the one or more fungal target nucleic acid amplicons is detected using a 5′ capture probe and a 3′ capture probe set forth in Table 8 or Table 9.

68. The method of any one of claims 1-67, wherein the panel is a panel shown in any one of Tables 20-24 or in Table 27.

69. The method of claim 68, wherein the panel comprises:
(i) a first subpanel comprising the following pathogen target nucleic acids: Pan Gram negative, E. coli, K. pneumoniae, Enterobacter spp., Enterobacter cloacae complex, Citrobacter spp., S. marcescens, P. mirabilis, Salmonella spp., and an internal control;
(ii) a second subpanel comprising the following pathogen target nucleic acids: Acinetobacter spp., A. baumanii, P. aeruginosa, S. maltophilia, H. influenzae, KPC, NDM/VIM/IMP, OXA-48-like, CTX-M 14/15, and an internal control;
(iii) a third subpanel comprising the following pathogen target nucleic acids: Pan Gram positive, Enterococcus spp., E. faecium, E. faecalis, Staphylococcus spp., S. aureus, coagulase negative Staphylococcus spp., mecA/C, vanA/B, and an internal control;
(iv) a fourth subpanel comprising the following pathogen target nucleic acids: Streptococcus spp., S. pneumoniae, S. pyogenes, S. agalactiae, Viridans Group Streptococcus, Anaerobes, Corynebacterium spp., ermA/B, mefA/E, and an internal control; and
(v) a fifth subpanel comprising the following pathogen target nucleic acids: Candida spp., C. albicans, C. tropicalis, C. parapsilosis, C. krusei, C. glabrata, C. auris, Aspergillus spp., Cryptococcus spp., and an internal control.

70. The method of claim 68, wherein the panel comprises Pan Gram Positive, Pan Gram Negative, Staphylococcus aureus, Coagulase negative staphylococci, Enterococcus spp., Enterococcus faecium, Enterococcus faecalis, Streptococcus pneumoniae, Streptococcus agalactiae, Streptococcus pyogenes, Clostridium spp., Mycobacterium spp., Enterobacterales, Escherichia coli, Klebsiella pneumoniae, Klebsiella aerogenes, Enterobacter cloacae complex, Citrobacter spp., Serratia spp., Proteus spp., Acinetobacter baumannii, Bacteroides spp., Haemophilus influenzae, Pseudomonas aeruginosa, Stenotrophomonas maltophilia, mecA, mecC, vanA/B, mefA/E, KPC, NDM, VIM, IMP, OXA-48, OXA-23, OXA-24/40, CTX-M, AmpC, mcr-1, and strA/strB.

71. The method of any one of claims 1-70, wherein amplifying is in the presence of whole blood proteins and non-target nucleic acids.

72. The method of any one of claims 2-71, wherein lysing comprises mechanical lysis or heat lysis.

73. The method of claim 72, wherein the mechanical lysis is beadbeating or sonicating.

74. The method of any one of claims 1-73, wherein the steps of the method are completed within 5 hours.

75. The method of claim 74, wherein the steps of the method are completed within 4 hours.

76. The method of claim 75, wherein the steps of the method are completed within 3 hours.

77. The method of any one of claims 1-76, wherein the method detects a pathogen target nucleic acid of a pathogen present at a concentration of 10 cells/mL of biological sample or less.

78. The method of claim 77, wherein the method detects a pathogen target nucleic acid of a pathogen present at a concentration of 3 cells/mL of biological sample.

79. The method of claim 78, wherein the method detects a pathogen target nucleic acid of a pathogen present at a concentration of 1 cells/mL of biological sample.

80. The method of any one of claims 1-79, wherein the method results in redundant detection of the pathogen at the pan level, genus level, species level, and/or resistance level.

81. The method of any one of claims 1-80, wherein the method identifies the pathogen at the pan level.

82. The method of any one of claims 1-81, wherein the method identifies the pathogen at the genus level.

83. The method of any one of claims 1-82, wherein the method identifies the pathogen at the species level.

84. The method of any one of claims 1-83, wherein the method identifies the pathogen at the resistance level.

85. The method of any one of claims 1-84, wherein the amplifying comprises polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM).

86. The method of claim 85, wherein the amplifying comprises PCR.

87. The method of claim 86, wherein the PCR is symmetric PCR or asymmetric PCR.

88. The method of any one of claims 1, 2, and 4-87, wherein the detecting comprises magnetic, sequencing, optical, fluorescent, mass, density, chromatographic, and/or electrochemical detection.

89. The method of claim 88, wherein the detecting comprises T2 magnetic resonance (T2MR).

90. The method of any one of claims 1-89, wherein the detecting comprises sequencing.

91. The method of claim 90, wherein the sequencing comprises massively parallel sequencing, Sanger sequencing, or single-molecule sequencing.

92. The method of claim 91, wherein the massively parallel sequencing comprises sequencing by synthesis or sequencing by ligation.

93. The method of claim 92, wherein the massively parallel sequencing comprises sequencing by synthesis.

94. The method of claim 92 or 93, wherein the sequencing by synthesis comprises ILLUMINA™ dye sequencing, ion semiconductor sequencing, or pyrosequencing.

95. The method of claim 94, wherein the sequencing by synthesis comprises ILLUMINA™ dye sequencing.

96. The method of claim 92, wherein the sequencing by ligation comprises sequencing by oligonucleotide ligation and detection (SOLiD™) sequencing or polony-based sequencing.

97. The method of claim 91, wherein the single-molecule sequencing is nanopore sequencing, single-molecule real-time (SMRT™) sequencing, or Helicos™ sequencing.

98. The method of any one of claims 3-14 and 16-97, wherein:
(i) each assay sample is contacted with 1×10⁶ to 1×10¹³ magnetic particles per milliliter of the biological sample;
(ii) step (g) comprises measuring the T₂ relaxation response of the assay sample, and wherein increasing agglomeration in the assay sample produces an increase in the observed T₂ relaxation time of the assay sample;
(iii) the magnetic particles have a mean diameter of from 600 nm to 1200 nm;
(iv) the magnetic particles have a T₂ relaxivity per particle of from 1×10⁹ to 1×10¹² mM⁻¹s⁻¹; and/or
(v) the magnetic particles are substantially monodisperse.

99. The method of claim 98, wherein the magnetic particles have a mean diameter of from 650 nm to 950 nm.

100. The method of claim 99, wherein the magnetic particles have a mean diameter of from 670 nm to 890 nm.

101. A method for identifying a patient infected with a pathogen, the method comprising:
(a) providing a biological sample obtained from the subject; and
(b) detecting the presence of a pathogen target nucleic acid in the biological sample according to the method of any one of claims 1-100,
wherein the presence of a pathogen target nucleic in the biological sample obtained from the subject identifies the subject as one who may be infected with the pathogen.

102. The method of claim 101, further comprising selecting an optimized therapy for the patient based on the presence of the pathogen and/or the presence of one or more resistance genes.

103. The method of any one of claims 1-102, further comprising administering the optimized therapy to the patient.

104. The method of any one of claims 101-103, wherein the method results in administration of an optimized therapy to the patient faster than standard of care.

105. The method of any one of claims 101-104, wherein the method results in de-escalation of empiric therapy within 3 to 5 hours.
